(19) United States
(12) Patent Application Publication
(10) Pub. No.: US 2021/0353611 A1
(43) Pub. Date: Nov. 18, 2021

(54) METHODS OF TREATING AMYOTROPHIC LATERAL SCLEROSIS

(71) Applicant: Yeda Research and Development Co. Ltd., Rehovot (IL)

(72) Inventors: Eran ELINAV, Mazkeret Batya (IL); Eran SEGAL, Ramat-HaSharon (IL)

(73) Assignee: Yeda Research and Development Co. Ltd., Rehovot (IL)

(21) Appl. No.: 17/277,441

(22) PCT Filed: Sep. 19, 2019
(86) PCT No.: PCT/IL2019/051041

§ 371 Date: Mar. 18, 2021

(30) Foreign Application Priority Data
Sep. 20, 2018 (IL) 261908
Jun. 27, 2019 (IL) 267752

(51) Int. Cl. A61K 31/455; A61K 35/74; A61P 25/28

(52) U.S. Cl. CPC A61K 31/455; A61P 25/28; A61K 35/74

(57) ABSTRACT

A method of treating ALS in a subject in need thereof is disclosed. The method comprises administering to the subject a therapeutically effective amount at least two metabolites.

### RELATED APPLICATION/S

This application claims the benefit of priority of Israel Patent Application No. 261908 filed on Sep. 20, 2018 and Israel Patent Application No. 267752 filed on Jun. 27, 2019, the contents of which are incorporated herein by reference in their entirety.

The contents of the above applications are all incorporated by reference as if fully set forth herein in their entirety.

### SEQUENCE LISTING STATEMENT

The ASCII file, entitled 78818 Sequence Listing.txt, created on Sep. 19, 2019, comprising 22,138 bytes, submitted concurrently with the filing of this application is incorporated herein by reference.

### FIELD AND BACKGROUND OF THE INVENTION

The present invention, in some embodiments thereof, relates to methods of treating Amyotrophic Lateral Sclerosis (ALS) and, more particularly, but not exclusively, to treatment with bacterial populations or metabolites thereof.

Amyotrophic Lateral Sclerosis (ALS) is a progressive, idiopathic neurodegenerative disorder characterized by premature death of motor neurons and an average survival rate of 3-5 years from diagnosis. The majority of ALS cases are sporadic (sALS), while 10-20% of cases are familial (fALS), and driven by genetic mutations in genes such as superoxide dismutase 1 (SOD1). Extensive efforts are being made to develop ALS-targeting drugs like edaravone, but none so far has yielded a conclusively effective disease-modifying activity. While past epidemiological studies did not identify clear environmental factors correlating with ALS occurrence and severity, the Central Nervous System (CNS) is increasingly recognized to be influenced by peripheral signals, such as circulatory small molecular-weight metabolites which may be absorbed from the GI tract to the blood stream and reach the CNS through the brain-blood barrier (BBB), where they can modulate metabolic, transcriptional and epigenetic programs in neurons and in other resident cells.

The gut microbiome, a microbial ecosystem impacting multiple host physiological functions, is a large potential source of such potentially bioactive CNS disease-modulating metabolites. Indeed, accumulating evidence suggests that the composition and function of the gut microbiome play significant roles in the pathogenesis of neurological disorders such as autism, Parkinson's disease, Alzheimer's disease, Multiple sclerosis and epileptic seizures. Metabolites secreted, depleted or modified by the gut microbiome were shown to participate in neuronal transmission, synaptic plasticity, myelination and host complex behaviors. Several hints suggest that the host-gut microbiome interface may be potentially involved in the course of ALS. A disrupted Intestinal barrier accompanied by lower levels of colonic tight-junction protein Zonula occludens-1 (ZO-1) and the adherence protein E-cadherin were reported in 2 month-old SOD1-Tg mice, potentially leading to dysbiosis hallmarked by a reduction in the butyrate producing bacteria Butyrivibrio fibrisolvens. Butyrate administration to SOD1-Tg mice altered their microbiome composition, although microbiome assessment was performed at a single time point and 3 animals per group, thereby precluding accurate assessment of the scope, significance, and mechanism of dysbiosis at this setting. 16S rDNA analysis of ALS patients yielded conflicting results, with one study noting a dysbiotic configuration in 6 ALS patients compared to 5 healthy controls, while another showing no significant compositional differences between 25 ALS patients and 32 healthy controls. No direct functional microbiome investigation has been performed in this setting.

Background art includes Richard Bedlack & The ALSUntangled Group (2018) ALSUntangled 42: Elysium health's “basis”, Amyotrophic Lateral Sclerosis and Frontotemporal Degeneration, 19:3-4,317-319, DOI: 10.1080/21678421.2017.1373978; and Harlan et al, 2016, The Journal of Biological Chemistry 291, 10836-10846.

### SUMMARY OF THE INVENTION

According to an aspect of the present invention there is provided a method of treating ALS in a subject in need thereof comprising administering to the subject a therapeutically effective amount of at least two metabolites, wherein at least one of the at least two metabolites is selected from the group consisting of propyl 4-hydroxybenzoate, triethanolamine, serotonin, 2-keto-3-deoxy-gluconate, nicotinamide, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2sahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE thereby treating ALS.

According to an aspect of the present invention there is provided a use of at least two metabolites for treating ALS, wherein at least one of the at least two metabolites are selected from the group consisting of propyl 4-hydroxybenzoate, triethanolamine, serotonin, 2-keto-3-deoxy-gluconate, nicotinamide, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE.

According to an aspect of the present invention there is provided a method of treating ALS in a subject in need thereof comprising administering to the subject a therapeutically effective amount of a probiotic comprising a bacterial population selected from the group consisting of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, Akkermansia Muciniphila (AM), Anaeroplasma, Prevotella, Distanosis, Parabacteroides, Rikenellaceae, Alistipes, Candidatus Arthromitus, Eggerthella, Oscillibacter, Subdoligranulum and Lactobacillus, thereby treating ALS.

According to an aspect of the present invention there is provided a use of a probiotic for treating ALS, wherein the probiotic comprises a bacterial population selected from the group consisting of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, Akkermansia Muciniphila (AM), Anaeroplasma, Prevotella, Distanosis, Parabacteroides, Rikenellaceae, Alistipes, Candidatus Arthromitus, Eggerthella, Oscillibacter, Subdoligranulum and Lactobacillus.

According to an aspect of the present invention there is provided a method of treating ALS in a subject in need thereof comprising administering to the subject a therapeutically effective amount of an agent that selectively decreases the amount of a bacterial population selected from the group consisting of Escherichia coli, Clostridium leptum, Ruminococcus gnavus, Clostridium nexile, Clostridium bolteae, Bacteroides fragilis, Catenibacterium mitsuokai, Bifidobacterium dentium, Megasphaera, Parasutterella excrementihominis, Burkholderiales bacterium, Clostridium ramosum, Streptococcus anginosus, Flavonifractorplautii, Methanobrevibacter_smithii, Acidaminococcus intestine, Ruminococcus_torques, Ruminococcus, Bifidobacterium, Coriobacteriaceae, Bacteroides, Parabacteroides, S24_7, Clostridiaceae, flavefaciens, Desulfovibrioaceae, Allobaculum, Sutterella, Helicobacteraceae, Coprococcus, Oscillospira in the gut microbiome of the subject, thereby treating the ALS.

According to an aspect of the present invention there is provided a use of an agent that selectively decreases the amount of a bacterial population selected from the group consisting of Escherichia coli, Clostridium leptum, Ruminococcus gnavus, Clostridium nexile, Clostridium bolteae, Bacteroides fragilis, Catenibacterium mitsuokai, Bifidobacterium dentium, Megasphaera, Parasutterella excrementihominis, Burkholderiales bacterium, Clostridium ramosum, Streptococcus anginosus, Flavonifractor_plautii, Methanobrevibacter_smithii, Acidaminococcus intestine, Ruminococcus_torques, Ruminococcus, Bifidobacterium, Coriobacteriaceae, Bacteroides, Parabacteroides, S24_7, Clostridiaceae, flavefaciens, Desulfovibrioaceae, Allobaculum, Sutterella, Helicobacteraceae, Coprococcus, Oscillospira for treating ALS.

According to an aspect of the present invention there is provided a method of treating ALS in a subject in need thereof comprising administering to the subject a therapeutically effective amount of a metabolite selected from the group consisting of propyl 4-hydroxybenzoate, triethanolamine, serotonin, 2-keto-3-deoxy-gluconate, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE thereby treating ALS.

According to an aspect of the present invention there is provided a use of a metabolite selected from the group consisting of propyl 4-hydroxybenzoate, triethanolamine, serotonin, 2-keto-3-deoxy-gluconate, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE for treating ALS.

According to an aspect of the present invention there is provided a method of diagnosing ALS of a subject comprising analyzing microbial metabolites of the subject, wherein a statistically significant decrease in abundance of a microbial metabolite selected from the group consisting of propyl 4-hydroxybenzoate, triethanolamine, serotonin, 2-keto-3-deoxy-gluconate, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18: 1/16:0), sphingomyelin (d18:1/18:1, d18:2/18:0), pyruvate, taurocholate, N-acetyltyrosine, tauro-beta-muricholate, tauroursodeoxycholate phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE compared to the abundance of the microbial metabolite in a healthy subject is indicative of ALS and/or a statistically significant increase in abundance of a microbial metabolite selected from the group consisting of taurourcholate compared to the abundance of the microbial metabolite in a healthy subject is indicative of ALS.

According to an aspect of the present invention there is provided a method of diagnosing ALS of a subject comprising analyzing the amount and/or activity of Ruminococcus in a microbiome of the subject, wherein a statistically significant increase in abundance and/or activity of Ruminococcus compared to its abundance in the microbiome of a healthy subject is indicative of ALS.

According to embodiments of the present invention, at least one of the at least two metabolites is selected from the group consisting of nicotinamide, phenol sulfate, equol sulfate and cinnamate.

According to embodiments of the present invention, at least one of the at least two metabolites is selected from the group consisting of propyl 4-hydroxybenzoate, triethanolamine, serotonin, 2-keto-3-deoxy-gluconate nicotinamide, N-trimethyl 5-aminovalerate, phenylalanylglycine, theobromine, cys-gly, glutamate and 1-palmitoyl-2-docosahexaenoyl-GPC.

According to embodiments of the present invention, the at least two metabolites are selected from the group consisting of propyl 4-hydroxybenzoate, triethanolamine, serotonin, 2-keto-3-deoxy-gluconate nicotinamide, N-trimethyl 5-aminovalerate, phenylalanylglycine, theobromine, cys-gly, glutamate and 1-palmitoyl-2-docosahexaenoyl-GPC.

According to embodiments of the present invention, at least one of the at least two metabolites is nicotinamide.

According to embodiments of the present invention, at least one of the at least two metabolites is comprised in a bacterial population.

According to embodiments of the present invention, the bacterial population is selected from the group consisting of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, Akkermansia Muciniphila (AM), Anaeroplasma, Prevotella, Distanosis, Parabacteroides, Rikenellaceae, Alistipes, Candidatus Arthromitus, Eggerthella, Oscillibacter, Subdoligranulum and Lactobacillus.

According to embodiments of the present invention, the bacterial population comprises Akkermansia Muciniphila (AM).

According to embodiments of the present invention, the bacterial population comprises Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum and Anaerostipes hadrus.

According to embodiments of the present invention, the bacterial population is selected from the group consisting of Ruminococcus, Desulfovibrioaceae, Allobaculum, Sutterella, Helicobacteraceae, Coprococcus and Oscillospira.

According to embodiments of the present invention, the bacterial population is selected from the group consisting of Escherichia coli, Clostridium leptum, Ruminococcus gnavus, Clostridium nexile, Clostridium bolteae, Bacteroides fragilis, Catenibacterium mitsuokai, Bifidobacterium dentium, Megasphaera, Parasutterella excrementihominis, Burkholderiales bacterium, Clostridium ramosum, Streptococcus anginosus, Flavonifractor_plautii, Methanobrevibacter_smithii, Acidaminococcus intestine and Ruminococcus_torques.

According to embodiments of the present invention, the bacterial population comprises Ruminococcus.

According to embodiments of the present invention, the Ruminococcus comprises Ruminococcus torques or Ruminococcus gnavus.

According to embodiments of the present invention, the agent is an antibiotic.

According to embodiments of the present invention, the agent is a bacteriophage.

According to embodiments of the present invention, the Ruminococcus comprises Ruminococcus torques or Ruminococcus gnavus.

According to embodiments of the present invention, the method further comprises analyzing the amount and/or activity of at least one of the bacteria selected from the group consisting of Escherichia coli, Clostridium leptum, Clostridium nexile, Clostridium bolteae, Bacteroides fragilis, Catenibacterium mitsuokai, Bifidobacterium dentium, Megasphaera, Parasutterella excrementihominis, Burkholderiales bacterium, Clostridium ramosum, Streptococcus anginosus, Flavonifractorplautii, Methanobrevibacter_smithii and Acidaminococcus intestine, wherein a statistically significant increase in abundance of the bacteria compared to its abundance in the microbiome of a healthy subject is indicative of ALS.

According to embodiments of the present invention, the method further comprises analyzing the amount and/or activity of at least one of the bacteria selected from the group consisting of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, wherein a statistically significant decrease in abundance of the bacteria compared to its abundance in the microbiome of a healthy subject is indicative of ALS.

According to embodiments of the present invention, the analyzing comprises analyzing a sample of a microbiome of the subject.

According to embodiments of the present invention, the microbiome is selected from the group consisting of a gut microbiome, an oral microbiome, a bronchial microbiome, a skin microbiome and a vaginal microbiome.

According to embodiments of the present invention, the microbiome is a gut microbiome.

According to embodiments of the present invention, the sample comprises a fecal sample.

According to embodiments of the present invention, the analyzing is effected in a blood sample of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-K. Antibiotic treatment exacerbates motor symptoms in an ALS mouse model. (A) Experimental design. Evaluation of motor symptoms by behavioral (B) rotarod, (C) hanging-wire grip tests and (D) neurological scoring across the disease course. *P<0.05, **P<0.005, Mann-Whitney U test. The experiment was repeated 3 times, (N=5-10 mice). (E) Histological images and (F) quantification of lower-motor neurons in the spinal cords of 140-day old water- and Abx-treated SOD1-Tg mice. *P<0.05, Mann-Whitney U test. (G) T₂ maps and (H-I) quantification of T₂ relaxation time in the corresponding areas between water- and Abx-treated SOD1-Tg mice throughout the disease progression. **P<0.005, ***P<0.0005, Mann-Whitney U test. The experiment was repeated twice, (N=5 mice). (J) Survival of GF (N=14) and SPF (N=17) SOD1-Tg mice. **P<0.005, Log-rank test. The experiment was repeated twice (K). Survival of Abx- and water-treated TDP43-Tg (N=10 in each group) mice. ****P<0.0001, Log-rank test. The experiment was repeated twice.

FIGS. 2A-H. SOD1-Tg mice develop early gut microbiome compositional and functional differences as compared to WT littermate controls. Weighted UniFrac PCoA on (A) day 40 (pre-symptomatic), (B) day 100 (disease onset) and (C) day 140 (advanced disease). The experiment was repeated 3 times, (N=6 mice in each group). (D) Species-level taxa summary obtained by gut microbiome metagenomic shotgun sequencing of WT and SOD1-Tg stool samples during disease progression. (E) PCA of KEGG entries of WT and SOD1-Tg microbiome. p=1.57×10⁻¹⁴, Spearman correlation coefficient. (F) Schematic representation and (G) heatmap of bacterial gene abundances of tryptophan metabolism. (H) Heatmap of bacterial gene abundances of the nicotinamide and nicotinate biosynthesis pathway. N=6 mice, *P<0.05, **P<0.005, ***P<0.0005, Mann-Whitney U test.

FIGS. 3A-H. Akkermansia muciniphila colonization ameliorates motor degeneration and increases life-span in SOD1-Tg mice. (A) Linear regression of AM relative abundance (16S rDNA sequencing) of SOD1-Tg and WT stool over time and (B) qPCR of AM 16S gene copies in fecal DNA extract (N=6 mice). Motor functions of SOD1-Tg and WT mice treated with AM indicated by (C) rotarod, (D) hanging-wire grip test and (E) neurological scoring. (F) Histological images and (G) spinal cord motor neuron quantification in 140-day old PBS- and AM-treated SOD1-Tg mice. *P<0.05, **P<0.005 Mann-Whitney U test. (H) Survival of PBS-, AM-, Prevotella melaninogenica (PM)- and Lactobacillus gasseri (LG)-treated mice ***P<0.0005 Log-rank test. The experiment was repeated 6 times, (N=5-26 mice).

FIGS. 4A-F. Akkermansia muciniphila treatment is associated with enhanced nicotinamide biosynthesis in SOD1-Tg mice. (A) Significantly increased serum metabolites in SOD1-Tg mice treated with AM (upper-right quadrant N=7-8 mice). (B) Serum levels nicotinamide pathway metabolites in SOD1-Tg and WT mice treated with AM or PBS. (C) Nicotinamide levels in bacterial cultures. **P<0.005, ***P<0.0005 Mann-Whitney U test. CSF nicotinamide levels of SOD1-Tg and WT mice treated with AM or PBS on (D) day 100 and (E) day 140. *P<0.05, **P<0.005, ***P<0.0005 Mann-Whitney U test. (F) Schematic representation of the microbiome-derived nicotinamide producing genes in AM treated SOD1-Tg fecal samples. The indicated genes increased in abundance following AM treatment (N=7-8 mice), Mann Whitney U ranksum test.

FIGS. 5A-G. Nicotinamide treatment ameliorates ALS progression in SOD1-Tg mice. (A) CSF and (B) sera NAM levels in NAM and vehicle treated SOD1-Tg mice (N=10 mice). Motor performances of NAM or vehicle treated SOD1-Tg mice using subcutaneous osmotic pumps indicated by (C) rotarod, (D) hanging-wire grip test and (E) neurological scoring. *P<0.05 ***P<0.0005 Mann-Whitney U test. The experiment was repeated 3 times, (N=10 mice). (F) Survival assessment of NAM and vehicle treated SOD1-Tg mice p=0.1757, Log-rank test. (G) neurological scoring of Abx-pretreated SOD1-Tg mice inoculated with WT or ΔnadA E. coli. ***P<0.0005 Mann-Whitney U test.

FIGS. 6A-E. Uncovering potential downstream motor neuron modulatory mechanisms of AM and NAM treatments. (A) Heatmap of FDR-corrected differentially-expressed genes in the spinal cords of NAM-treated SOD1-Tg mice (N=10 mice). (B) Spearman correlation of spinal cord transcripts log2 fold change between AM- and NAM-treated SOD1-Tg mice. (C) Comparison of the significantly differentially-expressed genes following NAM treatment with the KOG database classified into 4 neuropathological groups. FDR-corrected gene set enrichment distribution of spinal cord transcripts of (D) NAM-treated and (E) AM-treated SOD1-Tg mice into biological process, molecular functions and cellular components.

FIGS. 7A-F. Microbiome-derived nicotinamide metabolism is impaired in ALS patients (A) PCA of bacterial species composition (for PCI p=3.3×10⁻⁶, Spearman correlation coefficient) or (B) KEGG orthology (KO) annotated bacterial genes (for PC1 p=2.8×10⁻⁹, Spearman correlation coefficient) obtained by metagenomic shotgun sequencing of stool samples from ALS patients (N=32) and healthy controls (family members, N=27). (C) KO relative abundances of microbiome-associated genes of the nicotinamide pathway in ALS and healthy stool samples. (D) Serum metabolites levels of tryptophan/nicotinamide pathways in ALS patients and healthy individuals obtained by non-targeted metabolomics. (E) Serum and (F) CSF NAM levels of ALS patients (N=41 for serum and 12 for CSF) and healthy controls (N=21 for serum and 17 for CSF), ***P<0.0005, Mann Whitney U test.

FIGS. 8A-I. Antibiotic treatment exacerbates ALS symptoms in SOD1-Tg mice. SOD1-Tg and WT littermate control mice were untreated or treated with broad-spectrum Abx in their drinking water from age 40 days until the experimental end-point. On days 60, 80, 100, 120 and 140 motor performances of the mice were assessed by (A, D and G) rotarod, (B, E and H) hanging wire grip test and (C, F and I) neurological scoring. (N=5-10 mice), *P<0.05, **P<0.005, Mann-Whitney U test.

FIGS. 9A-P. The effects of antibiotic treatment on ALS symptoms in SOD1-Tg mice. Linear regression of motor functions over time in SOD1-Tg and WT treated indicated by (A) rotarod, (B) hanging-wire grip test, and (C) neurological score. (D) MRI of brain areas and their corresponding (E-I) quantification of T2 relaxation time between water and Abx-treated SOD1-Tg mice throughout ALS. *P<0.05, **P<0.005, ****P<0.00005, Mann-Whitney U test. (J) Home cage locomotion analysis over a period of 46 h, days 100-101 (N=5 mice). *P=0.03. Distributions of immune cell sub-populations in the small-intestine (K-L), colon (M-N), spinal cord on day 50 (O) and 140 (P) between water and Abx-treated SOD1-Tg mice. (N=5 mice), Mann-Whitney U test.

FIGS. 10A-D. Survival of GF- vs. SPF-SOD1-Tg mice and Abx-treated TDP43-Tg mice. Survival of SPF- and GF-SOD1-Tg mice that were spontaneously colonized on day 115. *P<0.05, Log-rank test. The experiment was done twice: (A) (N=13 SPF- and 6 GF SOD1-Tg mice) and (B) (N=5 SPF- and 8 GF-SOD1-Tg mice). (C-D) Survival of Abx- and water-treated TDP43-Tg mice **P<0.005, ****P<0.0001 Log-rank test. The experiment was done twice (N=5-10 mice in each group).

FIGS. 11A-O. Microbial compositional dynamics in the SOD1-Tg mouse model across

ALS progression. (A) Taxa summary of bacterial phyla in individual WT and SOD1-Tg mice during ALS course and (B) genera (averaged time points) obtained by 16S rDNA sequencing of stool samples. (N=6 mice), the experiment was repeated 3 times. (C) Relative abundances of significant differentially representative genera between SOD1-Tg and WT mice across the disease progression. (D-M) FDR-corrected linear regression comparison of representative bacterial relative abundance change during ALS progression between WT and SOD1-Tg stool. Spearman correlation coefficient. (N) Alpha diversity of SOD1-Tg and WT microbiomes over time. The experiment was repeated 3 times, (N=6 mice in each group. (O) qPCR-based quantification of total 16S copy-number in 1 ng of DNA extracted from stool samples of SOD1-Tg and WT mice (N=5-6 mice).

FIGS. 12A-M. Microbial compositional dynamics in Abx-treated SOD1-Tg mouse model across ALS progression. (A) Taxa summary of bacterial phyla in individual Abx-treated WT and SOD1-Tg mice during ALS course. Weighted UniFrac PCoA on (B) day 47 (pre-Abx), and (C-G) days 60-140 of the disease under chronic Abx regime. (H-M) FDR corrected volcano plots of significantly enriched bacterial genera of Abx-treated WT and SOD1-Tg mice during ALS course.

FIGS. 13A-I. Microbial spontaneous colonization in Ex-GF SOD1-Tg mouse model across ALS progression. (A) Taxa summary of bacterial genera in individual Ex-GF WT and SOD1-Tg undergoing spontaneous bacterial colonization during ALS course. (B-E) Weighted UniFrac PCoA of Ex-GF WT and SOD1-Tg mice on days 4, 5, 53 and 63 following spontaneous colonization. (F-I) FDR corrected volcano plots of significantly enriched bacterial genera of Ex-GF WT and SOD1-Tg during ALS course on days 4, 5, 53 and 63 following spontaneous colonization.

FIGS. 14A-E. A vivarium-affected dysbiosis in the SOD1-Tg mouse model (A) Weighted UniFrac PCoA and (B) Alpha diversity of WT and SOD1-Tg mice housed in a different non-barrier vivarium (vivarium B, Ben-Gurion University) on weeks 4, 6, 8 and 12 of age. (C) Individual and (D) averaged taxa summary of bacterial genera in 80 days old WT mice at vivarium A (Weizmann Institute of Science) and vivarium B (Ben-Gurion University). (E) Abundance percentage summary of the top 20 highly abundant microbiome genera in WT animals at the two facilities and their corresponding abundances in SOD1-Tg animals. The comparison has performed once, (N=5-8) mice in each group.

FIGS. 15A-N. Metagenomic differences between WT and SOD1-Tg fecal microbiomes (A) PCoA plot of bacterial composition and (B) Taxa summary representation at the species level of gut microbiome of WT and SOD1-Tg mice obtained by metagenomic shotgun sequencing. The experiment was repeated twice (N=6 mice). (C-N) FDR-corrected linear regression comparison of representative bacterial relative abundance change during ALS progression between WT and SOD1-Tg stool. Spearman correlation coefficient.

FIGS. 16A-L. Metabolic measurements in SOD1-Tg and WT littermates Representative recording (A, C, E, G, I, J, K) and quantification (B, D, F, H, L) of food intake (A, B), water consumption (C, D), respiratory exchange ratio (E, F), O₂ consumption (G, H), Heat production (I), locomotion (J) and speed (K, L) of 60 days old WT (N=8) and SOD1-Tg (N=7) mice.

FIGS. 17A-L. Mono-colonization of Abx pre-treated SOD1-Tg mice with selected ALS-correlating microbiome strains. Motor functions of Abx pre-treated SOD1-Tg mice treated with PBS, Eggerthella lento (EL), Coprobacillus cateniformis (CC), Parabacteroides goldsteinii (PG), Lactobacillus murinus (LM), Parabacteroides distasonis (PD), Lactobacillus gasseri (LG), Prevotella melaninogenica (PM), or Akkermansia muciniphila (AM, ATCC 835) indicated by (A) rotarod, (B) hanging-wire grip test and (C) neurological scoring. (D-F) Motor functions of Abx pre-treated SOD1-Tg mice treated with PBS or Eisenbergiella tayi (ET), or (G-I) Subdoligranulum variabile (SV). (J-L) Motor functions of Abx pre-treated WT littermate controls treated with PBS, LM, PD, LG, PM or AM. (N=6-8 mice) *P<0.05, **P<0.005, ***P<0.0005 Mann-Whitney U test.

FIGS. 18A-M. The effects of Ruminococcus torques mono-colonization on ALS progression in SOD1-Tg mice. (A) Linear regression of Ruminococcus torques (RT) relative abundance (16S rDNA sequencing) of SOD1-Tg and WT stool (N=6 mice). (B) Rotarod, (C) hanging-wire grip test and (D) neurological scoring of Abx-pretreated WT and SOD1-Tg treated with PBS or RT (N=5-9 mice), *P<0.05, **P<0.005, ***P<0.0005, Mann-Whitney U test. (E) Histological images and (F) quantification of spinal cord motor neurons of 140 days old PBS- and RT-treated SOD1-Tg mice. (G) Brain areas and their corresponding (H-M) T₂ relaxation time quantification between PBS and RT-treated SOD1-Tg mice throughout the disease. *P<0.05, **P<0.005, ***P<0.0005, ****P<0.00005 Mann-Whitney U test. The experiment was repeated twice, (N=5 mice).

FIGS. 19A-I. Ruminococcus torques treatment exacerbates ALS symptoms in SOD1-Tg mice. Assessment of Abx-pretreated SOD1-Tg and WT littermate treatment with Ruminococcus torques (RT) in three biological repeats, by (A, D and G) rotarod, (B, E and H) hanging wire grip test and (C, F and I) neurological scoring. (N=5-10 mice), *P<0.05, **P<0.005, ***P<0.0005 Mann-Whitney U test.

FIGS. 20A-O. Akkermansia muciniphila treatment attenuates ALS symptoms in SOD1-Tg mice. Abx-pretreated SOD1-Tg and WT littermate control mice were treated orally with AM (ATCC 835) or PBS as vehicle from age 60 days until the experimental end-point. On days 60, 80, 100, 120 and 140 motor performance of the mice was assessed by (A, D, G, J and O) rotarod, (B, E, H, K and M) hanging-wire grip test and (C, F, I, L and N) neurological scoring. (N=5-26 mice), *P<0.05, **P<0.005, ***P<0.0005, Mann-Whitney U test.

FIGS. 21A-L. The effects of Akkermansia muciniphila treatment on ALS manifestation and microbiome composition in SOD1-Tg mice. (A-D) T2 relaxation time quantification in PBS and AM (ATCC 835)-treated Abx-pretreated SOD1-Tg mice at days 100 and 140. ***P<0.0005, ****P<0.00005, Mann-Whitney U test. (E) Systemic FITC-dextran measurement at 120 days WT and SOD1-Tg treated with PBS, AM, P. Melaninogenica (PM) or L. gaseri (LG). (F) PCoA of bacterial species compositions in SOD1-Tg mice treated with PBS or AM. (G) Genera bacterial summary of SOD1-Tg treated with PBS or AM. AM relative abundance in (H) SOD1-Tg or (I) WT mice treated with PBS or AM. *P<0.05, ***P<0.0005, ****P<0.00005, Mann-Whitney ranksum test. (I) Individual and (J) averaged qPCR-based fold change of Akkermansia muciniphila 16S copy number in mucosal and luminal samples across the GI tract of 140 days old AM or PBS treated WT and SOD1-Tg mice (K) Genera bacterial summary of SOD1-Tg or (L) WT mice treated with PBS or AM.

FIGS. 22A-C. Akkermansia muciniphila (ATCC 2869) treatment attenuates ALS symptoms in SOD1-Tg mice. Abx-pretreated SOD1-Tg and WT littermate control mice were treated orally with AM (ATCC 2869) or PBS as vehicle from age 60 days until the experimental end-point. On days 60, 80, 100, 120 and 140 motor performance of the mice was assessed by (A) rotarod, (B) hanging-wire grip test and (C) neurological scoring. (N=8-10 mice), **P<0.005, Mann-Whitney U test.

FIGS. 23A-J. Akkermansia muciniphila treatment alters mucus properties of SOD1-Tg mice. Immunohistochemical assessment of distal colon mucosa of 140 days old (A) PBS- and (B) AM- (BAA-835) Abx-pretreated WT and SOD1-Tg mice. DNA stained with Sytox-green (green) and the mucus with an anti-MUC2C3 antiserum and goat anti-Ig (red). The non-stained areas between the epithelium and outer mucus/luminal bacteria is the inner mucus layer, allows points to bacteria in this. Heatmap representation of (C) total mucus proteomic landscape and (D) AM-related peptides and (E-J) quantification of key representative mucus components. (N=4-8 mice), Mann-Whitney U test.

FIGS. 24A-G. Serum metabolomic profile is affected by antibiotics or AM treatment in ALS SOD1-Tg mice. Heatmap representation of serum metabolites of 100 days old (A) naïve SOD1-Tg and their WT littermates, (B) water or Abx-treated SOD1-Tg mice, (C) PBS or AM-treated SOD1-Tg mice. (D) Scoring of top six serum metabolites which significantly altered by Abx treatment in SOD1-Tg mice by their potential to originate of the gut microbiome. Motor performances of Phenol sulfate or vehicle treated SOD1-Tg mice using subcutaneous osmotic pumps indicated by (E) rotarod, (F) hanging-wire grip test and (G) neurological scoring.

FIGS. 25A-B. Tryptophan and Nicotinamide metabolism are affected by antibiotics or AM treatment in ALS SOD1-Tg mice. Non-targeted metabolomics assessment of typtophan metabolism of (A) water and Abx-treated or (B) PBS and AM-treated 100 days old SOD1-Tg mice.

FIGS. 26A-I. Nicotinamide treatment ameliorates ALS progression in SOD1-Tg mice. Motor performances of NAM or vehicle treated SOD1-Tg mice using subcutaneous osmotic pumps indicated by (A, D and G) rotarod, (B, E and H) hanging-wire grip test and (C, F and I) neurological scoring (N=10 mice). *P<0.05, **P<0.005, ***P<0.0005, ****P<0.00005, Mann-Whitney U test.

FIGS. 27A-C. Mono-inoculation of SOD1-Tg mice with gut commensal impaired in NAM production (A) Nicotinamide levels in WT or ΔnadA E. coli cultures. ***P<0.0005, Mann-Whitney U test. Motor performances of WT or ΔnadA E. coli-inoculated Abx-pretreated SOD1-Tg mice indicated by (B) rotarod and (C) hanging-wire grip test.

FIG. 28. NAM differentially expressed genes associated with Nuclear respiratory factor-1 (NRF-1). Representation of spinal cord transcripts obtained by RNA-seq analysis that changed similarly after AM and NAM treatments of SOD1-Tg mice and share the binding site for the Nuclear respiratory factor-1 (NRF-1) transcription factor. The analysis was done using the G:Profiler platform⁸⁵.

FIGS. 29A-B. Different gut microbiome composition and serum metabolites profile in ALS patients. (A) Taxa summary representation at the species level of gut microbiome of healthy family members and ALS patients obtained by metagenomic shotgun sequencing and a table of the top 20 changed bacterial species between ALS patients and healthy control individuals. (B) Top 97 differentially-represented serum metabolites between healthy individuals (N=13) and ALS patients (N=23) obtained by untargeted metabolomics.

### DESCRIPTION OF SPECIFIC EMBODIMENTS OF THE INVENTION

The present invention, in some embodiments thereof, relates to methods of treating Amyotrophic Lateral Sclerosis (ALS) and, more particularly, but not exclusively, to treatment with bacterial populations or metabolites thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Amyotrophic Lateral Sclerosis (ALS) is an idiopathic, genetically-influenced neurodegenerative disorder, whose variable onset and clinical course may be contributed by unknown environmental factors.

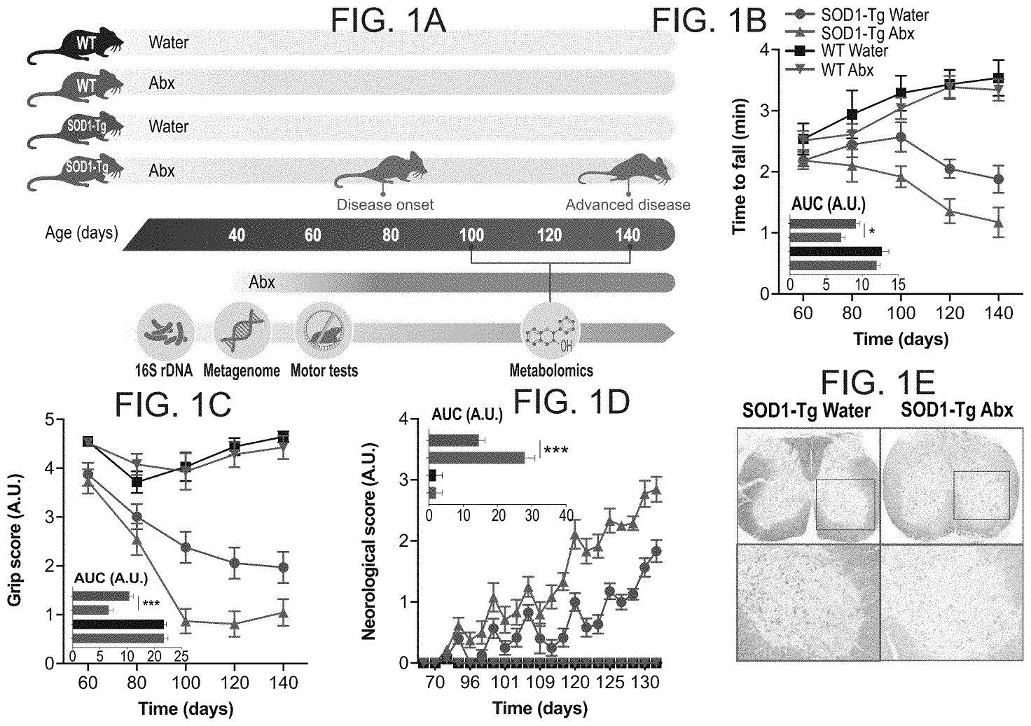

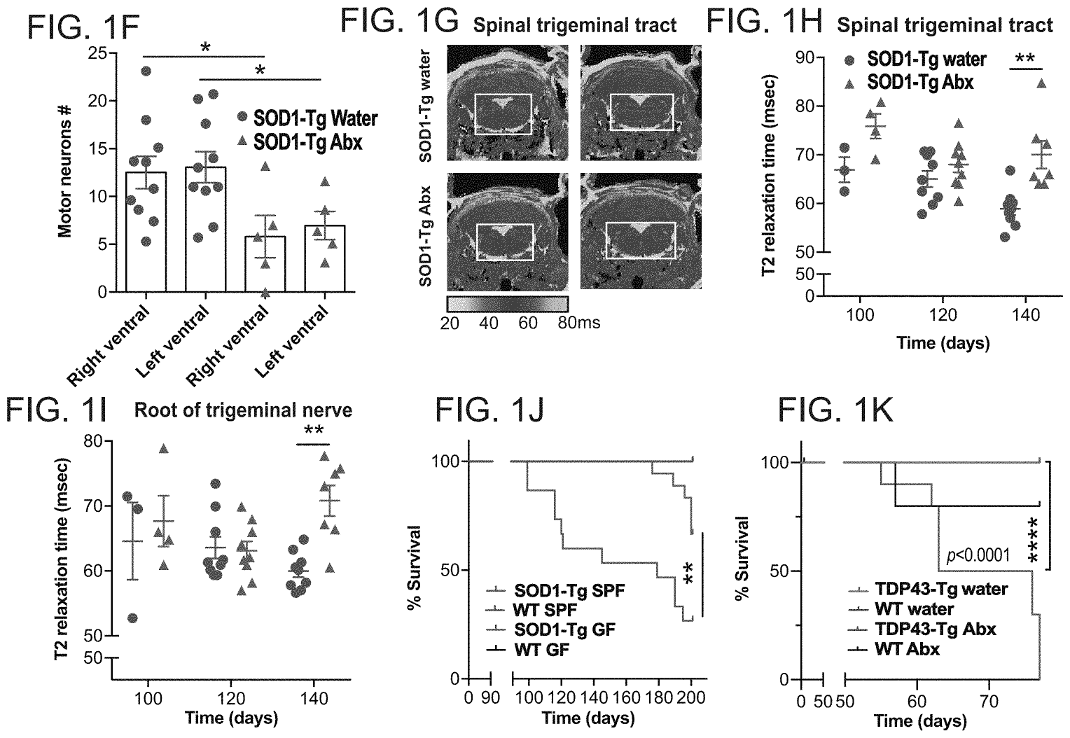

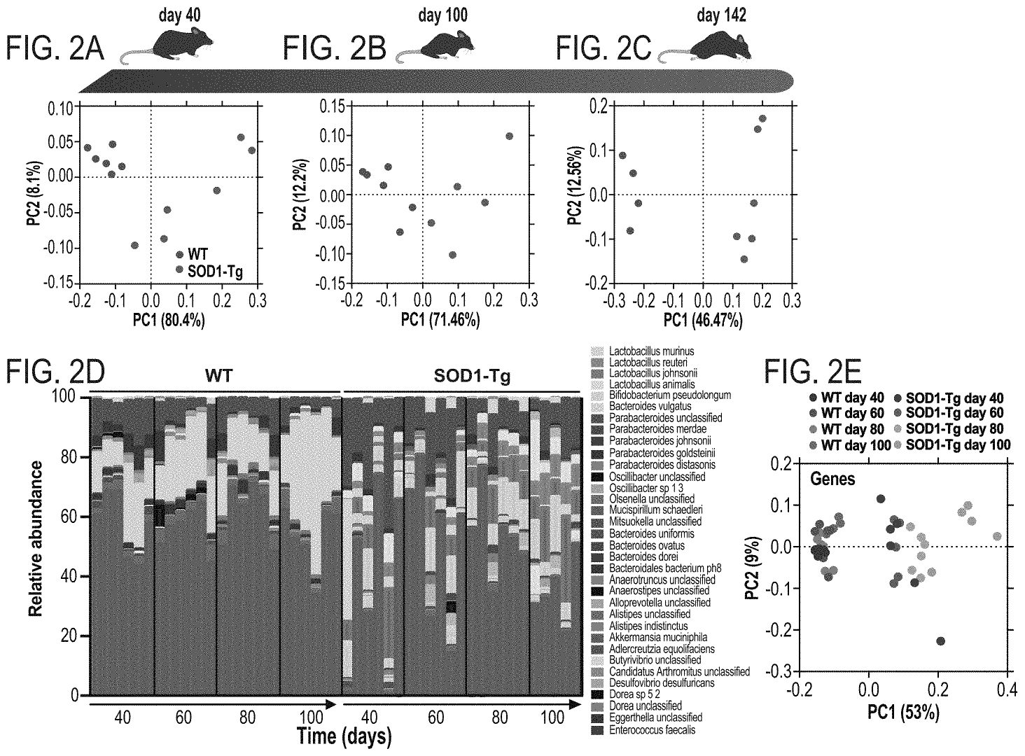

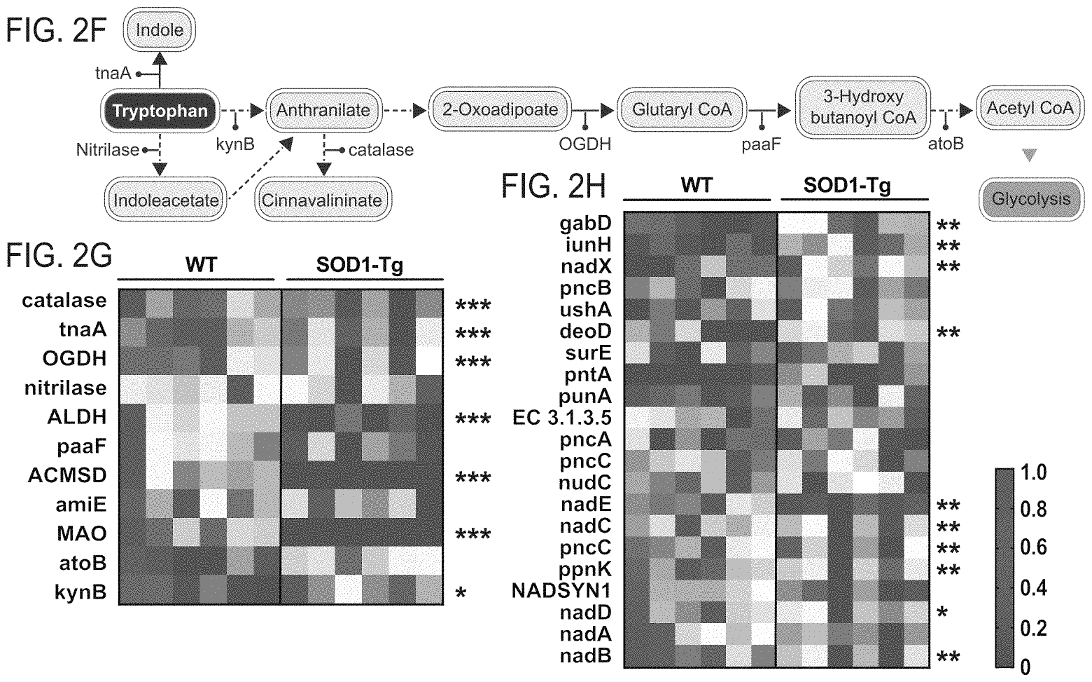

The present inventors have now demonstrated that wide spectrum antibiotics-induced depletion of the gut microbiome in the most commonly used ALS mouse model (the SOD1-Tg mouse model) leads to worsened disease symptoms (FIGS. 1A-K). Furthermore, the gut microbiome composition and metagenomic function of SOD1-Tg mice were altered compared to WT littermates, even before the onset of motor clinical symptoms, resulting in a markedly altered systemic metabolomic profile in these mice (FIGS. 2A-H).

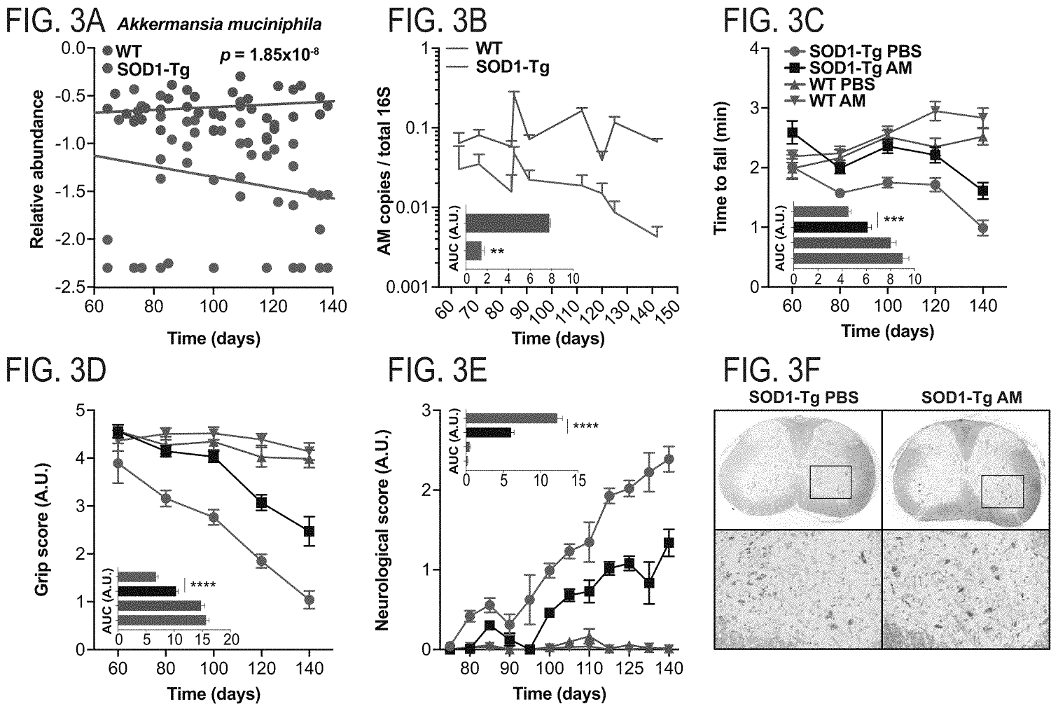

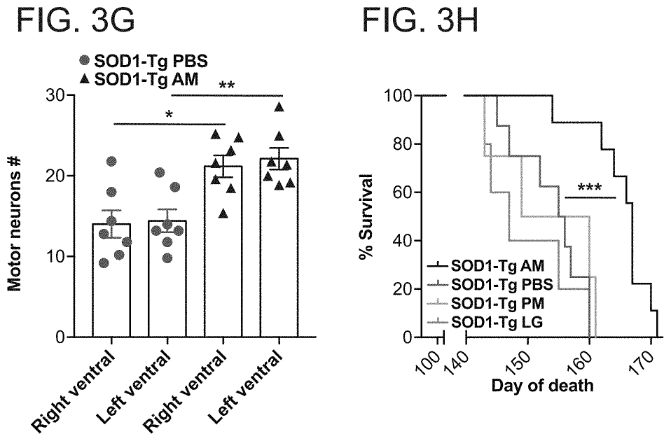

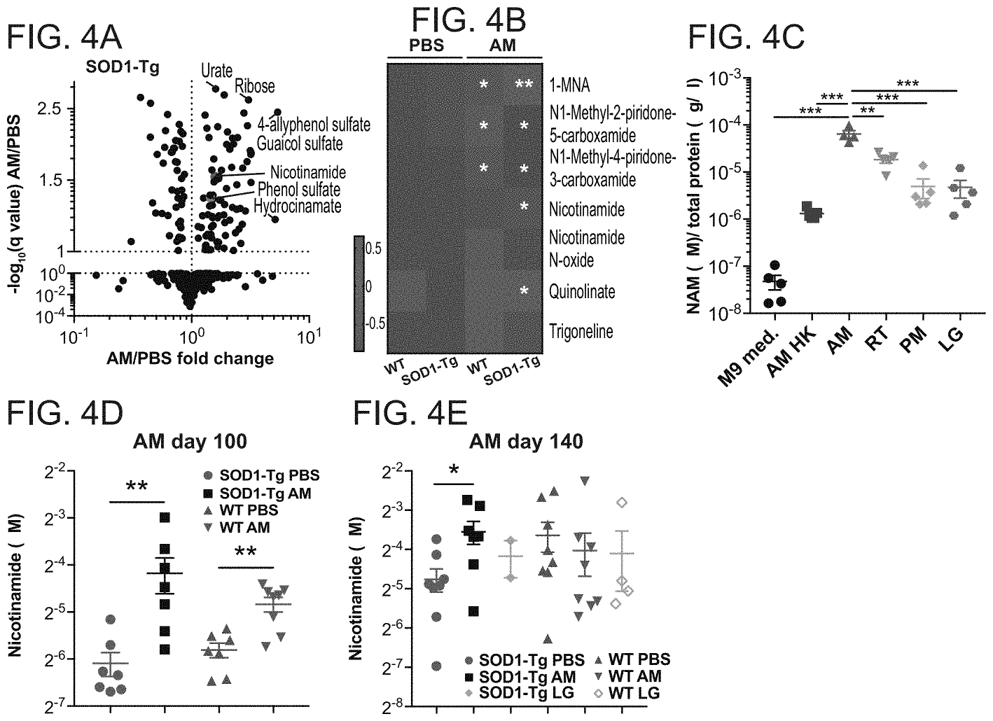

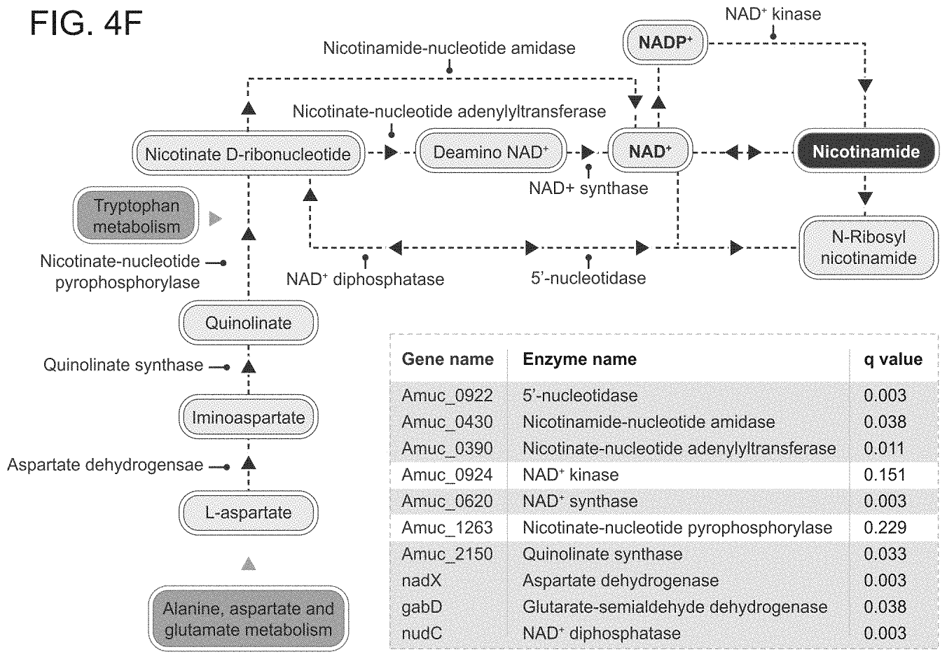

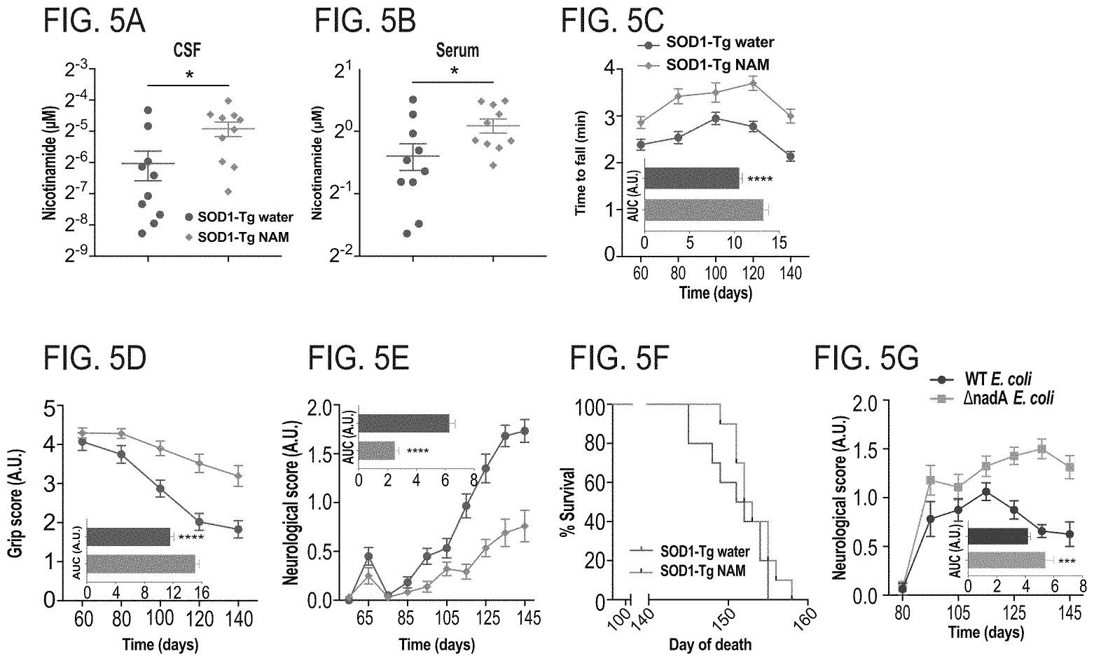

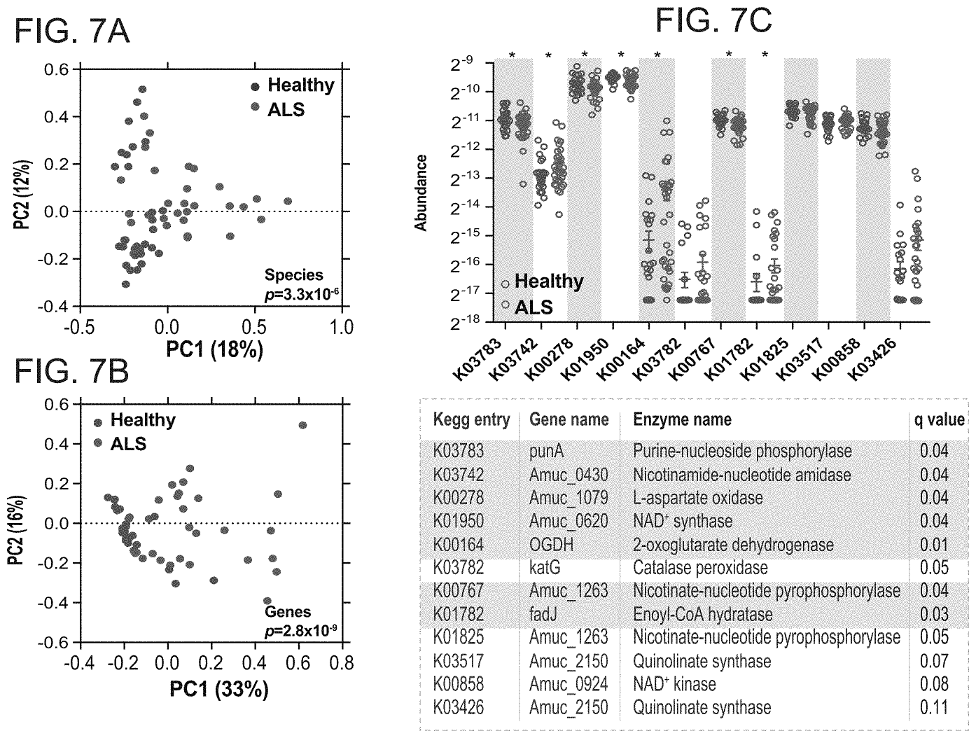

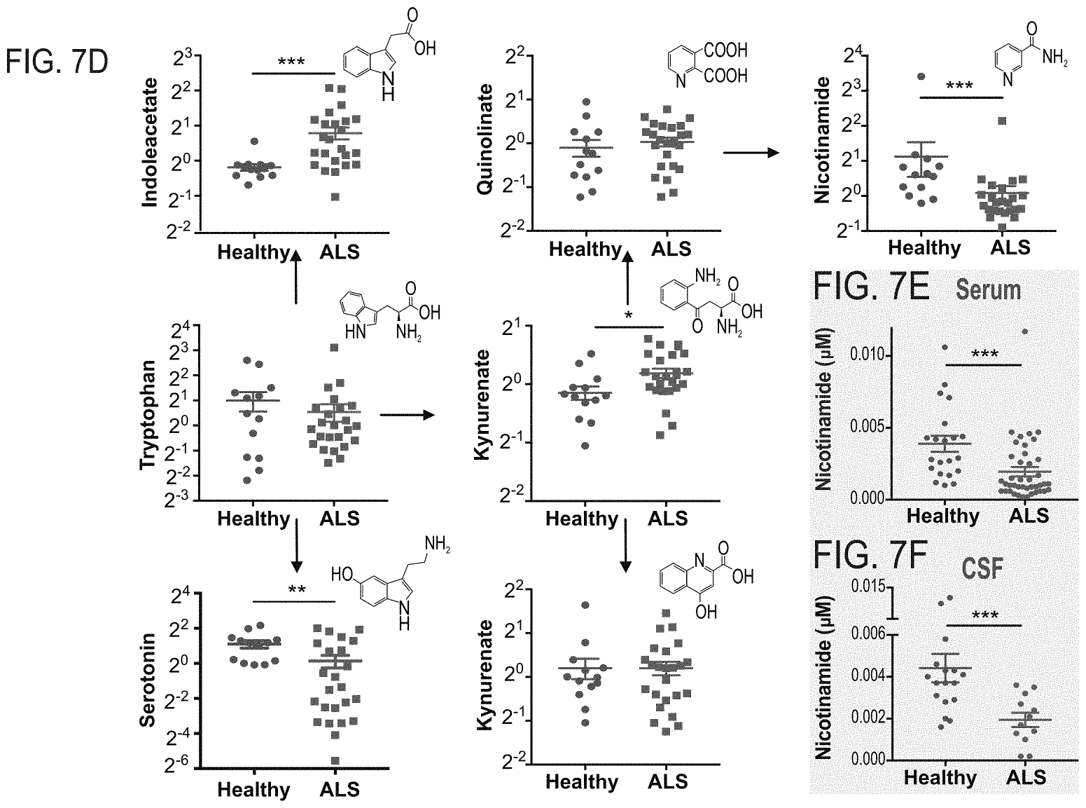

Several microbial species were identified to be correlated or anti-correlated with disease severity in SOD1-Tg mice. Of these, post-antibiotic colonization of SOD1-Tg with anaerobic mono-cultures of Akkermansia Muciniphila (AM) led to improved motor symptoms and survival (FIGS. 3A-H), while colonization with Ruminococcus was associated with worsening disease symptoms (FIGS. 14A-M and 15A-I). Furthermore, key AM-derived microbial genes of the Nicotinamide (NAM) biosynthetic pathway were enriched in the gut microbiome of AM-supplemented SOD1-Tg mice, while NA and its biosynthetic intermediates were enriched, in this setting, in the cerebrospinal fluid (CSF) and serum of AM-treated SOD1-Tg mice (FIGS. 4A-F). Moreover, systemic NAM supplementation of SOD1-Tg mice induced clinical improvement in motor neuron symptoms, coupled with distinct beneficial CNS transcriptomic modifications (FIGS. 5A-F and 6A-E). In humans, a dysbiotic gut microbiome metagenomic configuration, skewed serum metabolomic profile, and altered serum and CSF NAM levels were noted in ALS patients compared to healthy family controls (FIGS. 7A-E). Together, these results suggest that modulatory links may exist between distinct gut commensals, their modulated metabolites and motor manifestations in ALS animal models and potentially in humans.

Consequently, the present teachings suggest use of gut microbiome-associated modulating agents for the treatment of ALS.

Thus, according to a first aspect of the present invention, there is provided a method of treating ALS in a subject in need thereof comprising administering to the subject a therapeutically effective amount of a therapeutically effective amount of a metabolite selected from the group consisting of propyl 4-hydroxybenzoate, triethanolamine, serotonin, 2-keto-3-deoxy-gluconate, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE thereby treating ALS.

As used herein, the term “treating” includes abrogating, substantially inhibiting, slowing or reversing the progression of ALS, substantially ameliorating clinical or aesthetical symptoms of ALS or substantially preventing the appearance of clinical or aesthetical symptoms of ALS.

As used herein, the term “treating” refers to inhibiting, preventing or arresting the development of a pathology (i.e. ALS) and/or causing the reduction, remission, or regression of a pathology. Those of skill in the art will understand that various methodologies and assays can be used to assess the development of a pathology or reduction, remission or regression of a pathology, as further disclosed herein.

Amyotrophic lateral sclerosis (ALS), also known as Lou Gehrig's disease and Motor Neuron Disease (MND), is a progressive, fatal, neurodegenerative disease caused by the degeneration of motor neurons, the nerve cells in the central nervous system that control voluntary muscle movement. ALS typically causes muscle weakness and atrophy throughout the body as both the upper and lower motor neurons degenerate, ceasing to send messages to muscles. Unable to function, the muscles gradually weaken, develop fasciculations (twitches) because of denervation, and eventually atrophy because of that denervation. Affected subjects may ultimately lose the ability to initiate and control all voluntary movement; bladder and bowel sphincters and the muscles responsible for eye movement are usually, but not always, spared. Cognitive or behavioral dysfunction is also associated with the disease; about half of ALS subjects experience mild changes in cognition and behavior, and 10-15% show signs of frontotemporal dementia. Language dysfunction, executive dysfunction, and troubles with social cognition and verbal memory are the most commonly reported cognitive symptoms in ALS.

The term “ALS”, as used herein, includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking) and Progressive Muscular Atrophy (PMA, typically affecting only the lower motor neurons).

According to specific embodiments, ALS is classical ALS.

The term “ALS” includes sporadic and familial (hereditary) ALS, ALS at any rate of progression (i.e. rapid or slow progression) and ALS at any stage (e.g. prior to onset, at onset and late stages of ALS).

According to specific embodiments, ALS is sporadic ALS.

According to specific embodiments, ALS is familial ALS.

According to specific embodiments, ALS is rapid progression ALS.

As used herein, the phrase “rapid progression ALS” refers to ALS in which the symptoms progress continuously and significant degradation of motor neurons can be observed within less than a year with subject survival of up to 4 years from diagnosis. According to specific embodiments, the rapid progression ALS is characterized by a change of above 0.65 ALSFRS-R points over a period of 1 month.

According to specific embodiments, ALS is ALS-associated depression.

As used herein, the phrase “ALS-associated depression” refers to depression and/or anxiety which begin following ALS onset. According to specific embodiments, the ALS-associated depression is part of the ALS mechanism of action and may be attributed to e.g. Pseudo Bulbar Affect and frontal lobe dementia. Methods of diagnosing and monitoring depression are well known in the art and include, but not limited to, the ALS Depression Inventory (ADI-12), the Beck Depression Inventory (BDI); and the Hospital Anxiety Depression Scale (HADS) questionnaires.

As mentioned above, the method of the invention is directed, inter alia, to treating ALS.

The treatment may be initiated at any stage of the disease, including following detection of ALS symptoms.

Detection of ALS may be determined by the appearance of different symptoms depending on which motor neurons in the body are damaged first (and consequently which muscles in the body are damaged first). In general, ALS symptoms include the earliest symptoms which are typically obvious weakness and/or muscle atrophy. Other symptoms include muscle fasciculation (twitching), cramping, or stiffness of affected muscles, muscle weakness affecting an arm or a leg and/or slurred and nasal speech. Most ALS patients experience first symptoms in the arms or legs. Others first notice difficulty in speaking clearly or swallowing. Other symptoms include difficulty in swallowing, loss of tongue mobility and respiratory difficulties.

The symptoms may be also classified by the part of neuronal system that is degenerated, namely, upper motor neurons and lower motor neurons. Symptoms of upper motor neuron degeneration include tight and stiff muscles (spasticity) and exaggerated reflexes (hyperreflexia) including an overactive gag reflex. Symptoms of lower motor neuron degeneration include muscle weakness and atrophy, muscle cramps, and fleeting twitches of muscles that can be seen under the skin (fasciculations). To be diagnosed with ALS, patients must have signs and symptoms of upper and/or lower motor neuron damage that cannot be attributed to other causes.

Alternatively, treatment may be initiated at progressive stages of the disease, e.g. when muscle weakness and atrophy spread to different parts of the body and the subject has increasing problems with moving [e.g. the subject may suffer from tight and stiff muscles (spasticity), from exaggerated reflexes (hyperreflexia), from muscle weakness and atrophy, from muscle cramps, and/or from fleeting twitches of muscles that can be seen under the skin (fasciculations)], swallowing (dysphagia), speaking or forming words (dysarthria).

Method of monitoring ALS progression are well known in the art. Non-limiting examples of such methods include Physical evaluation by a physician; Weight; Electrocardiogram (ECG); ALS Functional Rating Scale (ALSFRS or ALSFRS-R) score; respiratory function which can be measured by e.g. vital capacity (forced vital capacity or slow vital capacity); muscle strength which can be measured by e.g. hand held dynamometry (HHD), hand grip strength dynamometry, manual muscle testing (MMT), electrical impedance myography (EIM) and Maximum Voluntary Isometric Contraction Testing (MVICT); motor unit number estimation (MUNE); cognitive/behavior function which can be measured by e.g. the ALS Depression Inventory (ADI-12), the Beck Depression Inventory (BDI) and the Hospital Anxiety Depression Scale (HADS) questionnaires; Quality of life which can be evaluated by e.g. the ALS Assessment Questionnaire (ALSAQ-40); and Akt phosphorylation and pAkt:tAkt ratio (see International Patent Application Publication No. WO2012/160563, the contents of which are fully incorporated herein by reference).

According to specific embodiments, the subject is monitored by ALS Functional Rating Scale (ALSFRS); respiratory function; muscle strength and/or cognitive function.

According to specific embodiments, muscle strength is evaluated by a method selected from the group consisting of hand held dynamometry (HHD), hand grip strength dynamometry, manual muscle testing (MMT) and electrical impedance myography (EIM); each possibility represents a separate embodiment of the present invention.

As used herein the term “subject” refers to a human subject at any age and of any gender which is diagnosed with a disease (i.e., ALS) or is at risk of to develop a disease (i.e. ALS).

According to specific embodiments, the subject has rapid progression ALS and/or ALS-associated depression.

According to specific embodiments the subject fulfils the El Escorial criteria for probable and definite ALS, i.e. the subject presents:

1. Signs of lower motor neuron (LMN) degeneration by clinical, electrophysiological or neuropathologic examination,

2. Signs of upper motor neuron (UMN) degeneration by clinical examination, and

3. Progressive spread of signs within a region or to other regions, together with the absence of:
Electrophysiological evidence of other disease processes that might explain the signs of LMN and/or UMN degenerations; and
Neuroimaging evidence of other disease processes that might explain the observed clinical and electrophysiological signs.

According to specific embodiments, the subject has an ALSFRS-R score of 26-42 prior to treatment according to the present invention.

According to specific embodiments, the subject has a disease progression rate greater than 0.65 ALSFRS-R points per month over the last 3-12 months prior to treatment according to the present invention.

As mentioned, the method includes administering to the subject a therapeutically effective amount of at least one of the following bacterial metabolites: propyl 4-hydroxybenzoate, triethanolamine, serotonin, 2-keto-3-deoxy-gluconate, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE.

According to a particular embodiment, at least one metabolite selected from the group consisting of propyl 4-hydroxybenzoate, triethanolamine, serotonin, 2-keto-3-deoxy-gluconate, N-trimethyl 5-aminovalerate, phenylalanylglycine, theobromine, cys-gly, glutamate, 1-palmitoyl-2-docosahexaenoyl-GPC are provided.

In another embodiment, the bacterial metabolite nicotinamide is provided together with one of the above mentioned metabolites.

In still another embodiment, the bacterial metabolite nicotinamide is not provided.

As used herein, the term “cinnamate” refers to cinnamic acid, salts thereof, cinnamate esters, p-dimethylaminocinnamate, cinnamaldehyde, cinnamyl acetate, cinnamyl alcohol, cinnamyl benzoate, cinnamyl cinnamate, cinnamyl formate, cinnamyl isobutyrate, cinnamyl isovalerate and cinnamyl phenylacetate and combinations thereof.

The equol of this aspect of the present invention may be (S)-equol (e.g. AUS-131, which is currently under development for treatment of hot flashes in menopausal women). In one embodiment, the equol is an equol salt such as equol sulfate.

Nicotinamide (NA), also known as “niacinamide”, is the amide derivative form of Vitamin B3 (niacin). NA has the chemical formula C₆H₆N₂O.

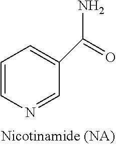

It will be understood by the skilled reader that nicotinamide, as well as other compounds used in the present invention, may be capable of forming salts, complexes, hydrates and solvates, and that the use of such forms in the defined treatments is contemplated herein. Nicotinamide preparations of high purities, e.g. of 97 or 99% purity, are commercially available. Such commercial preparations may suitably be used for preparing nicotinamide compositions for use in the present methods. Furthermore, synthesis methods of nicotinamide of high purity are known to those skilled in the art.

According to a particular embodiment, the nicotinamide is a nicotinamide derivative or a nicotinamide mimic. The term “derivative of nicotinamide (NA)” as used herein denotes a compound which is a chemically modified derivative of the natural NA. In one embodiment, the chemical modification may be a substitution of the pyridine ring of the basic NA structure (via the carbon or nitrogen member of the ring), via the nitrogen or the oxygen atoms of the amide moiety. When substituted, one or more hydrogen atoms may be replaced by a substituent and/or a substituent may be attached to a N atom to form a tetravalent positively charged nitrogen. Thus, the nicotinamide of the present invention includes a substituted or non-substituted nicotinamide. In another embodiment, the chemical modification may be a deletion or replacement of a single group, e.g. to form a thiobenzamide analog of NA, all of which being as appreciated by those versed in organic chemistry. The derivative in the context of the invention also includes the nucleoside derivative of NA (e.g. nicotinamide adenine). A variety of derivatives of NA are described, some also in connection with an inhibitory activity of the PDE4 enzyme (WO03/068233; WO02/060875; GB2327675A), or as VEGF-receptor tyrosine kinase inhibitors (WO01/55114). For example, the process of preparing 4-aryl-nicotinamide derivatives (WO05/014549). Other exemplary nicotinamide derivatives are disclosed in W001/55114 and EP2128244.

Nicotinamide mimics include modified forms of nicotinamide, and chemical analogs of nicotinamide which recapitulate the effects of nicotinamide in the differentiation and maturation of RPE cells from pluripotent cells. Exemplary nicotinamide mimics include benzoic acid, 3-aminobenzoic acid, and 6-aminonicotinamide. Another class of compounds that may act as nicotinamide mimics are inhibitors of poly(ADP-ribose) polymerase (PARP). Exemplary PARP inhibitors include 3-aminobenzamide, Iniparib (BSI 201), Olaparib (AZD-2281), Rucaparib (AG014699, PF-01367338), Veliparib (ABT-888), CEP 9722, MK 4827, and BMN-673.

In one embodiment, the nicotinamide is nicotinamide adenine dinucleotide (NAD). In another embodiment, the nicotinamide is nicotinamide riboside.

Exemplary doses of the bacterial metabolites described herein include 1 to 500 mg/kg daily. In one embodiment of the invention the treatment comprises the daily administration of >10 mg/kg, e.g. the daily administration of 10-500 mg/kg.

The present inventors contemplate combinations of the above described bacterial metabolites, e.g. two metabolites, three metabolites, four metabolites, five metabolites, six metabolites, seven metabolites, eight metabolites, nine metabolites or more.

Thus, for example the combination may include:

Nicotinamide and phenol sulfate;

Nicotinamide and equol;

Nicotinamide and cinnamate;

Nicotinamide, phenol sulfate and equol;

Nicotinamide, phenol sulfate and cinnamate;

Nicotinamide, equol and cinnamate;

Nicotinamide, equol, phenol sulfate and cinnamate.

Nicotinamide and at least one of the metabolites selected from the group consisting of propyl 4-hydroxybenzoate, triethanolamine, serotonin, 2-keto-3-deoxy-gluconate, N-trimethyl 5-aminovalerate, phenylalanylglycine, theobromine, cys-gly, glutamate and 1-palmitoyl-2-docosahexaenoyl-GPC.

The bacterial metabolite may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to one or more of the bacterial metabolites described herein accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

According to a particular embodiment, the agent is administered orally or rectally.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. nicotinamide) effective to prevent, alleviate or ameliorate symptoms of a disorder (e.g., ALS) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide blood, brain or CSF levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The metabolites of the present invention may be provided in a food (such as food bars, biscuits, snack foods and other standard food forms well known in the art), or in drink formulations. Drinks can contain flavoring, buffers and the like. Nutritional supplements comprising the metabolites of the present invention are also contemplated.

The metabolites of this aspect of the present invention may be provided via a probiotic composition comprising microbes that generate the metabolites.

The term “probiotic” as used herein, refers to one or more microorganisms which, when administered appropriately, can confer a health benefit on the host or subject and/or reduction of risk and/or symptoms of a disease (such as ALS), disorder, condition, or event in a host organism.

Thus, according to another aspect of the present invention there is provided a method of treating ALS comprising administering to the subject a therapeutically effective amount of a bacterial composition comprising at least one of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, Akkermansia Muciniphila (AM), Anaeroplasma, Prevotella, Distanosis, Parabacteroides (e.g. Parabacteroides distasonis, Parabacteroides goldsteinii) Rikenellaceae, Alistipes, Candidatus Arthromitus, Eggerthella, Oscillibacter, Subdoligranulum and Lactobacillus (e.g. Lactobacillus murinus).

According to a specific embodiment, the bacteria composition comprises at least one of, at least two of, at least three of, at least four of, at least five of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum and Anaerostipes hadrus.

According to a particular embodiment, the bacterial composition comprises Akkermansia Muciniphila (AM).

The probiotic microorganism may be in any suitable form, for example in a powdered dry form. In addition, the probiotic microorganism may have undergone processing in order for it to increase its survival. For example, the microorganism may be coated or encapsulated in a polysaccharide, fat, starch, protein or in a sugar matrix. Standard encapsulation techniques known in the art can be used. For example, techniques discussed in U.S. Pat. No. 6,190,591, which is hereby incorporated by reference in its entirety, may be used.

According to a particular embodiment, the probiotic composition is formulated in a food product, functional food or nutraceutical.

In some embodiments, a food product, functional food or nutraceutical is or comprises a dairy product. In some embodiments, a dairy product is or comprises a yogurt product. In some embodiments, a dairy product is or comprises a milk product.

In some embodiments, a dairy product is or comprises a cheese product. In some embodiments, a food product, functional food or nutraceutical is or comprises a juice or other product derived from fruit. In some embodiments, a food product, functional food or nutraceutical is or comprises a product derived from vegetables. In some embodiments, a food product, functional food or nutraceutical is or comprises a grain product, including but not limited to cereal, crackers, bread, and/or oatmeal. In some embodiments, a food product, functional food or nutraceutical is or comprises a rice product. In some embodiments, a food product, functional food or nutraceutical is or comprises a meat product.

Prior to administration, the subject may be pretreated with an agent which reduces the number of naturally occurring microbes in the microbiome (e.g. by antibiotic treatment). According to a particular embodiment, the treatment significantly eliminates the naturally occurring gut microflora by at least 20%, 30% 40%, 50%, 60%, 70%, 80% or even 90%.

In some particular embodiments, appropriate doses or amounts of probiotics to be administered may be extrapolated from dose-response curves derived from in vitro or animal model test systems. The effective dose or amount to be administered for a particular individual can be varied (e.g., increased or decreased) over time, depending on the needs of the individual. In some embodiments, where bacteria are administered, an appropriate dosage comprises at least about 100, 200, 300, 400, 500, 600, 700, 800, 900, 1000 or more bacterial cells. In some embodiments, the present invention encompasses the recognition that greater benefit may be achieved by providing numbers of bacterial cells greater than about 1000 or more (e.g., than about 1500, 2000, 2500, 3000, 35000, 4000, 4500, 5000, 5500, 6000, 7000, 8000, 9000, 10,000, 15,000, 20,000, 25,000, 30,000, 40,000, 50,000, 75,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 1×10⁸, 1×10⁹, 1×10¹⁰, 1×10¹¹, 1×10¹², 1×10¹³ or more bacteria.

The present inventors have further shown that levels of particular bacterial populations increase in the microbiome of a subject with ALS.

Thus, according to still another aspect of the present invention there is provided a method of treating ALS in a subject in need thereof comprising administering to the subject a therapeutically effective amount of an agent that selectively decreases the amount of a bacterial population selected from the group consisting of Escherichia coli, Clostridium leptum, Clostridium nexile, Clostridium bolteae, Bacteroides fragilis, Catenibacterium mitsuokai, Bifidobacterium dentium, Megasphaera, Parasutterella excrementihominis, Burkholderiales bacterium, Clostridium ramosum, Streptococcus anginosus, Flavonifractorplautii, Methanobrevibacter_smithii, Acidaminococcus intestine, Ruminococcus e.g. Ruminococcus_torques or Ruminococcus gnavus, Bifidobacterium, Coriobacteriaceae, Bacteroides, Parabacteroides, S24_7, Clostridiaceae, flavefaciens, Desulfovibrioaceae, Allobaculum, Sutterella, Helicobacteraceae, Coprococcus and Oscillospira, in the gut microbiome of the subject, thereby treating the ALS.

According to a particular embodiment, the bacterial population is selected from the group consisting of Escherichia coli, Clostridium leptum, Ruminococcus (e.g. Ruminococcus gnavus or Ruminococcus torques), Clostridium nexile, Clostridium bolteae, Bacteroides fragilis, Catenibacterium mitsuokai, Bifidobacterium dentium, Megasphaera, Parasutterella excrementihominis, Burkholderiales bacterium, Clostridium ramosum, Streptococcus anginosus, Flavonifractorplautii, Methanobrevibacter_smithii and Acidaminococcus intestine.

According to a particular embodiment, the bacterial population is selected from the group consisting of Ruminococcus, Desulfovibrioaceae, Allobaculum, Sutterella, Helicobacteraceae, Coprococcus and Oscillospira.

In a further embodiment, the bacterial population which is down-regulated is at least one of the following bacteria: Bacteroides dorei, Bacteroides vulgatus, Bacteroides xylanisolvens, Bifidobacterium pseudolongum, Dorea, Helicobacter_hepaticus, Lactobacillus_johnsonii, Lactobacillus_reuteri, Lactobacillus_sp_ASF360, Desulfovibrio_desulfuricans, Lactobacillus_vaginalis, Mucispirillum_schaedleri, Parabacteroides (e.g. Parabacteroides_johnsonii) and Ruminococcus_torques.

In one embodiment, at least two of the above described species/genus are down-regulated, at least three of the above described species/genus are down-regulated, at least four of the above described species/genus are down-regulated, at least five of the above described species/genus are down-regulated, all of the above described species or genus are down-regulated.

The present invention contemplates an agent which down-regulates at least one strain, 10% of the strains, 20% of the strains, 30% of the strains, 40% of the strains, 50% of the strains, 60% of the strains, 70% of the strains, 80% of the strains, 90% of the strains or all of the strains of the above disclosed species.

As used herein, the term “downregulates” refers to an ability to reduce the amount (either absolute or relative amount) and/or activity (either absolute or relative activity) of a particular species/genus of bacteria.

In one embodiment, the agent specifically downregulates the specified species/genus of bacteria.

Thus, for example, the agent may reduce the amount of the specified bacterial species/genus as compared to at least one other bacterial species/genus of the microbiome of the subject, by at least 2 fold. According to a particular embodiment, the agent downregulates the particular bacterial species/genus by at least 5 fold, 10 fold or more as compared to at least one other bacterial species/genus of the microbiome.

In another embodiment, the agent reduces the amount of the specified bacterial species/genus as compared to at least 10% of the total bacterial species/genus of the microbiome of the subject, by at least 2 fold. According to a particular embodiment, the agent downregulates the specified bacterial species/genus by at least 5 fold, 10 fold or more as compared to at least 10% of the total bacterial species/genus of the microbiome of the subject.

In another embodiment, the agent reduces the amount of the specified bacterial species/genus as compared to at least 20% of the total bacterial species/genus of the microbiome of the subject, by at least 2 fold. According to a particular embodiment, the agent downregulates the specified bacterial species/genus by at least 5 fold, 10 fold or more as compared to at least 20% of the total bacterial species/genus of the microbiome of the subject.

In another embodiment, the agent reduces the amount of the specified bacterial species/genus as compared to at least 30% of the total bacterial species/genus of the microbiome of the subject, by at least 2 fold. According to a particular embodiment, the agent downregulates the specified bacterial species/genus by at least 5 fold, 10 fold or more as compared to at least 30% of the total bacterial species/genus of the microbiome of the subject.

In another embodiment, the agent reduces the amount of the specified bacterial species/genus as compared to at least 40% of the total bacterial species/genus of the microbiome of the subject, by at least 2 fold. According to a particular embodiment, the agent downregulates the specified bacterial species/genus by at least 5 fold, 10 fold or more as compared to at least 40% of the total bacterial species/genus of the microbiome of the subject.

In another embodiment, the agent reduces the amount of the specified bacterial species/genus as compared to at least 50% of the total bacterial species/genus of the microbiome of the subject, by at least 2 fold. According to a particular embodiment, the agent downregulates the specified bacterial species/genus by at least 5 fold, 10 fold or more as compared to at least 50% of the total bacterial species/genus of the microbiome of the subject.

In another embodiment, the agent reduces the amount of the specified bacterial species/genus as compared to at least 60% of the total bacterial species/genus of the microbiome of the subject, by at least 2 fold. According to a particular embodiment, the agent downregulates the specified bacterial species/genus by at least 5 fold, 10 fold or more as compared to at least 60% of the total bacterial species/genus of the microbiome of the subject.

In another embodiment, the agent reduces the amount of the specified bacterial species/genus as compared to at least 70% of the total bacterial species/genus of the microbiome of the subject, by at least 2 fold. According to a particular embodiment, the agent downregulates the specified bacterial species/genus by at least 5 fold, 10 fold or more as compared to at least 70% of the total bacterial species/genus of the microbiome of the subject.

In another embodiment, the agent reduces the amount of the specified bacterial species/genus as compared to at least 80% of the total bacterial species/genus of the microbiome of the subject, by at least 2 fold. According to a particular embodiment, the agent downregulates the specified bacterial species/genus by at least 5 fold, 10 fold or more as compared to at least 80% of the total bacterial species/genus of the microbiome of the subject.

In another embodiment, the agent reduces the amount of the specified bacterial species/genus as compared to at least 90% of the total bacterial species/genus of the microbiome of the subject, by at least 2 fold. According to a particular embodiment, the agent downregulates the specified bacterial species/genus by at least 5 fold, 10 fold or more as compared to at least 90% of the total bacterial species/genus of the microbiome of the subject.

An exemplary agent which is capable of reducing a particular bacterial genus, species or strain is an antibiotic.

As used herein, the term “antibiotic agent” refers to a group of chemical substances, isolated from natural sources or derived from antibiotic agents isolated from natural sources, having a capacity to inhibit growth of, or to destroy bacteria, and other microorganisms, used chiefly in treatment of infectious diseases. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

According to a particular embodiment, the antibiotic is a non-absorbable antibiotic.

Other agents which are not antibiotics are also contemplated by the present inventors.

In one embodiment, the agent which is capable of down-regulating a particular bacterial genus/species/strain is a bacterial population that competes with the bacterial genus/species/strain for essential resources. Bacterial compositions are further described herein below.

In still another embodiment, the agent which is capable of down-regulating a particular bacterial genus/species/strain is a metabolite of a competing bacterial population (or even from the same species/strain) that serves to decrease the relative amount of the bacterial species/strain.

Additional agents that can specifically reduce a particular bacterial genus, species or strain are known in the art and include polynucleotide silencing agents.

Preferably, the polynucleotide silencing agent of this aspect of the present invention targets a sequence that encodes at least one essential gene (i.e., compatible with life) in the bacteria. The sequence which is targeted should be specific to the particular bacteria species that it is desired to down-regulate. Such genes include ribosomal RNA genes (16S and 23S), ribosomal protein genes, tRNA-synthetases, as well as additional genes shown to be essential such as dnaB, fabI, folA, gyrB, murA, pytH, metG, and tufA(B).

According to an embodiment of the invention, the polynucleotide silencing agent is specific to the target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

One agent capable of downregulating an essential bacterial gene is a RNA-guided endonuclease technology e.g. CRISPR system.

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

It will be appreciated that as well as treating ALS, the present inventors further propose testing particular bacterial species in the microbiome of the subject in order to diagnose the disease.

Thus, according to another aspect of the present invention there is provided a method of diagnosing ALS of a subject comprising analyzing the amount and/or activity of Ruminococcus in a microbiome of the subject, wherein a statistically significant increase in abundance and/or activity of Ruminococcus compared to its abundance in the microbiome of a healthy subject is indicative of ALS.

As used herein, the term “diagnosing” refers to determining the presence of a disease, classifying a disease, determining a severity of the disease (grade or stage), monitoring disease progression and response to therapy, forecasting an outcome of the disease and/or prospects of recovery.

Additional bacterial species/genus that may be analyzed that may aid in diagnosis include Akkermansia Muciniphila (AM), Anaeroplasma, Distanosis, Prevotella, Parabacteroides (e.g. Parabacteroides distasonis and Parabacteroides goldsteinii), Rikenellaceae, Alistipes, Candidatus Arthromitus, Eggerthella, Oscillibacter, Subdoligranulum, Lactobacillus (e.g. Lactobacillus murinus).

Additional bacterial species/genus that may be analyzed that may aid in diagnosis include Escherichia coli, Clostridium leptum, Clostridium nexile, Clostridium bolteae, Bacteroides fragilis, Catenibacterium mitsuokai, Bifidobacterium dentium, Megasphaera, Parasutterella excrementihominis, Burkholderiales bacterium, Clostridium ramosum, Streptococcus anginosus, Flavonifractor_plautii, Methanobrevibacter_smithii and Acidaminococcus intestine, wherein a statistically significant increase in abundance of the above mentioned bacteria compared to its abundance in the microbiome of a healthy subject is indicative of ALS.

Further bacterial species/genus that may be analyzed that may aid in diagnosis include Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, wherein a statistically significant decrease in abundance of the above mentioned bacteria compared to its abundance in the microbiome of a healthy subject is indicative of ALS.

The amount of the above bacterial species is typically decreased in a subject with ALS as compared to their abundance in the microbiome of a healthy subject.

The amount of the above bacterial species is typically increased in a subject with ALS as compared to their abundance in the microbiome of a healthy subject.

In order to diagnose a subject as having ALS, typically at least 1 (e.g. Ruminococcus), at least 2, at least 3, at least 4, at least 5, at least 6, at least 7, at least 8, at least 9 or even more of the above disclosed species/genus are analyzed.

Typically, the increase for any of the above described bacterial species/genus above a predetermined level is at least 1.5 times the amount, 2 times the amount, 3 times the amount, 4 times the amount, 5 times the amount as compared to the amount of that microbe in the microbiome of a healthy subject (e.g. subject not having ALS).

Typically, the decrease for any of the above described bacterial species/genus above a predetermined level is at least 1.5 times the amount, 2 times the amount, 3 times the amount, 4 times the amount, 5 times less the amount as compared to the amount of that microbe in the microbiome of a healthy subject (e.g. subject not having ALS).

It will be appreciated that when comparing abundance and/or activity of a particular bacterial species, care should be taken to compare between microbiomes of the same organ or tissue.

In one embodiment, the abundance of the above disclosed bacteria is analyzed.

Measuring a level or presence of a microbe may be effected by analyzing for the presence of microbial component or a microbial by product. Thus, for example the level or presence of a microbe may be effected by measuring the level of a DNA sequence. In some embodiments, the level or presence of a microbe may be effected by measuring 16S rRNA gene sequences or 18S rRNA gene sequences. In other embodiments, the level or presence of a microbe may be effected by measuring RNA transcripts. In still other embodiments the level or presence of a microbe may be effected by measuring proteins. In still other embodiments, the level or presence of a microbe may be effected by measuring metabolites.

Obtaining a Microbiome Sample

In order to analyze the microbiome, samples are taken from a subject.

The subject is typically a mammalian subject—e.g. human subject.

Thus, for example stool samples may be taken to analyze the gut microbiome, bronchial samples may be taken to analyze the bronchial microbiome, a saliva sample may be taken to analyze the oral microbiome etc. According to a particular embodiment, the microbiome of a subject is derived from a stool sample of the subject.

The present inventors have shown that changes in eating patterns (e.g. due to circadian misalignment) affect the composition of the microbiome. Therefore, preferably samples are taken at a fixed time in the day.

Obtaining chromosomal (genomic) DNA from microbiomes may be effected using conventional techniques, for example as disclosed in Sambrook and Russell, Molecular Cloning: A Laboratory Manual, cited supra. In some cases, particularly if small amounts of DNA are employed in a particular step, it is advantageous to provide carrier DNA, e.g. unrelated circular synthetic double-stranded DNA, to be mixed and used with the sample DNA whenever only small amounts of sample DNA are available and there is danger of losses through nonspecific binding, e.g. to container walls and the like.

In one embodiment, long fragments of chromosomal DNA are obtained. Cells are lysed and the intact nuclei may be pelleted with a gentle centrifugation step. The genomic DNA is then released (e.g. through proteinase K and RNase digestion, for several hours (e.g. 1-5 hours)). The material can be treated to lower the concentration of remaining cellular waste, e.g., by dialysis for a period of time (i.e., from 2-16 hours) and/or dilution. Since such methods need not employ many disruptive processes (such as ethanol precipitation, centrifugation, and vortexing), the genomic nucleic acid remains largely intact, yielding a majority of fragments that have lengths in excess of 150 kilobases. In some embodiments, the fragments are from about 5 to about 750 kilobases in lengths. In further embodiments, the fragments are from about 150 to about 600, about 200 to about 500, about 250 to about 400, and about 300 to about 350 kilobases in length.

Optionally, the target genomic DNA is then fractionated or fragmented to a desired size by conventional techniques including enzymatic digestion, shearing, or sonication, with the latter two finding particular use in the present invention.

Fragment sizes of the target nucleic acid can vary depending on the source target nucleic acid, and the library construction methods used, but for standard whole-genome sequencing such fragments may range from 50 to 600 nucleotides in length. In another embodiment, the fragments are 300 to 600 or 200 to 2000 nucleotides in length. In yet another embodiment, the fragments are 10-100, 50-100, 50-300, 100-200, 200-300, 50-400, 100-400, 200-400, 300-400, 400-500, 400-600, 500-600, 50-1000, 100-1000, 200-1000, 300-1000, 400-1000, 500-1000, 600-1000, 700-1000, 700-900, 700-800, 800-1000, 900-1000, 1500-2000, 1750-2000, and 50-2000 nucleotides in length. Longer fragments are also contemplated.

In a further embodiment, fragments of a particular size or in a particular range of sizes are isolated. Such methods are well known in the art. For example, gel fractionation can be used to produce a population of fragments of a particular size within a range of base-pairs, for example for 500 base pairs+50 base pairs.

In many cases, enzymatic digestion of extracted DNA is not required because shear forces created during lysis and extraction will generate fragments in the desired range. In a further embodiment, shorter fragments (1-5 kb) can be generated by enzymatic fragmentation using restriction endonucleases.

Quantifying Microbial Levels:

It will be appreciated that determining the abundance of microbes may be affected by taking into account any feature of the microbiome. Thus, the abundance of microbes may be affected by taking into account the abundance at different phylogenetic levels; at the level of gene abundance; gene metabolic pathway abundances; sub-species strain identification; SNPs and insertions and deletions in specific bacterial regions; growth rates of bacteria, the diversity of the microbes of the microbiome, as further described herein below.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differs between microbial species, the genes have highly conserved regions for primer binding. This specificity between conserved primer binding regions allows the rRNA genes of many different types of microbes to be amplified with a single set of primers and then to be distinguished by amplified sequences.

In some embodiments, a microbiota sample (e.g. fecal sample) is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QlAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, the abundance of any of the above described bacterial species/strain is determined by DNA sequencing.

Methods for sequence determination are generally known to the person skilled in the art. Preferred sequencing methods are next generation sequencing methods or parallel high throughput sequencing methods. For example, a bacterial genomic sequence may be obtained by using Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In some embodiments, determining a level or set of levels of one or more types of microbes comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

In some embodiments, determining a level or set of levels of one or more types of microbes comprises determining a level or set of levels of one or more microbial polypeptides. Methods of quantifying polypeptide levels are well known in the art and include but are not limited to Western analysis and mass spectrometry.

As mentioned herein above, as well as (or instead of) analyzing the abundance of microbes, the present invention also contemplates analyzing the level of microbial products.

Examples of microbial products include, but are not limited to mRNAs, polypeptides, carbohydrates and metabolites.

As used herein, a “metabolite” is an intermediate or product of metabolism. The term metabolite is generally restricted to small molecules and does not include polymeric compounds such as DNA or proteins. A metabolite may serve as a substrate for an enzyme of a metabolic pathway, an intermediate of such a pathway or the product obtained by the metabolic pathway.

In preferred embodiments, metabolites include but are not limited to sugars, organic acids, amino acids, fatty acids, hormones, vitamins, oligopeptides (less than about 100 amino acids in length), as well as ionic fragments thereof. Cells can also be lysed in order to measure cellular products present within the cell. In particular, the metabolites are less than about 3000 Daltons in molecular weight, and more particularly from about 50 to about 3000 Daltons.

The metabolite of this aspect of the present invention may be a primary metabolite (i.e. essential to the microbe for growth) or a secondary metabolite (one that does not play a role in growth, development or reproduction, and is formed during the end or near the stationary phase of growth.

Representative examples of metabolic pathways in which the metabolites of the present invention are involved include, without limitation, citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including, e.g., flavonoids and isoflavonoids), isoprenoids (including, e.g., terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs.

In some embodiments, levels of metabolites are determined by mass spectrometry. In some embodiments, levels of metabolites are determined by nuclear magnetic resonance spectroscopy, as further described herein below. In some embodiments, levels of metabolites are determined by enzyme-linked immunosorbent assay (ELISA). In some embodiments, levels of metabolites are determined by colorimetry. In some embodiments, levels of metabolites are determined by spectrophotometry.

According to a particular embodiment, the abundance of at least one of the following metabolites is analyzed: propyl 4-hydroxybenzoate, triethanolamine, serotonin, 2-keto-3-deoxy-gluconate, nicotinamide, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE.

According to a particular embodiment, the amount of nicotinamide is analyzed.

According to another embodiment, the metabolite is selected from the group consisting of propyl 4-hydroxybenzoate, triethanolamine, serotonin, 2-keto-3-deoxy-gluconate, nicotinamide, N-trimethyl 5-aminovalerate, phenylalanylglycine, theobromine, cys-gly, glutamate and 1-palmitoyl-2-docosahexaenoyl-GPC.

In order to diagnose a subject as having ALS, typically at least 1 (e.g. nicotinamide), at least 2, at least 3, at least 4, at least 5, at least 6, at least seven, at least eight, at least nine or more of the above disclosed metabolites is analyzed.

Typically, the increase for any of the above described metabolites above a predetermined level is at least 1.5 times the amount, 2 times the amount, 3 times the amount, 4 times the amount, 5 times the amount as compared to the amount of that metabolite in the microbiome of a healthy subject (e.g. subject not having ALS).

Typically, the decrease below a predetermined level is at least 1.5 times lower, 2 times lower, 3 times lower, 4 times lower, 5 times lower the amount as compared to the amount of that metabolite in the microbiome of a healthy subject (e.g. subject not having ALS).

As mentioned, as well as (or instead of) determining the abundance of the specified microbial species/strains for diagnosis of ALS, the present inventors also contemplate analyzing the growth dynamics of the microbes of the microbes of the microbiome.

The term “growth dynamics” refers to the growth phase of a bacterium (e.g. lag phase, stationary phase, exponential growth, death phase) and to the growth rate itself.

Measuring growth dynamics can be effected using the method described in WO 2016/079731, the contents of which are incorporated herein by reference.

Other methods of analyzing bacterial growth dynamics are known in the art and include for example analysis of optical density of a bacterial inoculant over a period of time.

Once a positive diagnosis has been made, additional tests may be carried out to corroborate the diagnosis—e.g. imaging, muscle biopsy etc. The subject may be treated following the diagnosis—e.g. using the bacterial populations/metabolites described herein, or by any other known gold-standard treatment for ALS.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice

G93A mSOD1-Tg mice on a C57BL/6 background were used. In all experiments, age- and gender-matched mice were used and WT littermates as controls. Mice were 40 days of age at the beginning of experiments. All mice were kept at a strict 24 hr reverse light-dark cycle, with lights being turned on from 10 pm to 10 am. Tryptophan-deficient diet (A10033Yi, Research diets, NJ, USA) was applied from the age of 40 days until the experimental end-point. For antibiotic treatment, mice were given a combination of vancomycin (0.5 g/l), ampicillin (1 g/l), kanamycin (1 g/l), and metronidazole (1 g/l) in their drinking water from the age of 40 days as previously described (Levy et al., 2015). For the Akkermansia muciniphila or Ruminococcus torques colonization, the 40 day old mice were treated with antibiotics for two weeks and following 2 days of wash period were gavaged with 200 μl of PBS-suspended bacteria (O.D.=0.7) weekly until the experimental end-point.

Administration of Metabolites

For the in vivo administration of NAM and Phenol sulfate, the Alzet osmotic minipumps model 1004 (Charles River) were used (infusing the compound at a rate of 0.11 μL/hour for 4 weeks). The pumps were filled with 100 μL 50 mg/ml Nicotinamide (Cymit Quimica, Barcelona, Spain) or 33.33 mg/ml Phenol sulfate sodium salt (TLC, Ontario, Canada) diluted in sterile water (equivalent to 49.28 mg/kg/week of NAM and 30.8 mg/kg/week Phenol sulfate). Vehicle control pumps contained equivalent volume of Ultra-pure water. 6-week-old SOD1-Tg and WT littermates mice were anesthetized by i.p. injection of ketamine (100 mg/kg) and xylazine (10 mg/kg), the neck skin was shaved and sterilized with 70% ethanol, 1 cm incision was made in the skin, the osmotic minipumps were inserted following minimal blunt dissection and placed above the right hind flank. The cut was then closed with sterile surgical clips and the animals were carefully monitored for any signs of stress, bleeding, pain, or abnormal behavior. The minipumps were replaced every 4 weeks for three times until the mice were 5 months old.

Assessment of Motor Functions in Mice

Rotarod: To assess motor coordination and balance, each mouse was tested with a rotarod device (Panlab Le8500 Harvard Apparatus, Spain), in acceleration speed mode (increasing from 4 rpm to 40 rpm during 10 min), with a maximum test time of 5 min. The mice were habituated on the horizontal rotating rod and pre-trained for 3 trials before the formal tests. Each mouse was recorded three times at the ages of 60, 80, 100, 120 and 140 days. The apparatus automatically recorded the elapsed time when the mouse fell from the spindle.

Hanging wire grip test: Mice are allowed to grip with their forepaws a 2 mm thick horizontal metal wire (suspended 80 cm above the working surface) and the latency to successfully raise their hind legs to grip the wire is recorded. The mice are observed for 30 sec and scored as follows—0=falls off within 10 sec.; 1=hangs onto bar by two forepaws; 2=attempts to climb onto bar; 3=hangs onto bar by two forepaws plus one or both hind paws; 4=hangs by all four paws plus tail wrapped around bar; 5=active escape to the end of bar.

Neurological scoring: Mice were neurologically scored by a system developed by ALS TDI (Hatzipetros et al., 2015): Score of 0: Full extension of hind legs away from lateral midline when mouse is suspended by its tail, and mouse can hold this for two seconds, suspended two to three times. Score of 1: Collapse or partial collapse of leg extension towards lateral midline (weakness) or trembling of hind legs during tail suspension. Score of 2: Toes curl under at least twice during walking of 12 inches, or any part of foot is dragging along cage bottom/table. Score of 3: Rigid paralysis or minimal joint movement, foot not being used for generating forward motion. Score of 4: Mouse cannot right itself within 30 sec after being placed on either side.

Home-cage locomotion: The locomotion of animals was quantified over a period of 46 h in the home cage, by automated sensing of body-heat image using an InfraMot (TSE-Systems). Individual animal movements were summed up every 30 min.

Survival

From the age of 130 days, mice were monitored daily. The endpoint was defined by reaching neurological score of 4 and/or more than 15% reduction in body weight. The probability of survival was calculated using the Kaplan-Meier method, and statistical analysis was performed using a log-rank test.

Cerebrospinal Fluid (CSF) Extraction

Mice were anesthetized by i.p. injection of ketamine (100 mg/kg) and xylazine (10 mg/kg). The skin of the neck was shaved, and the mouse was placed prone on the stereotaxic instrument. The head was secured with the head adaptors. The surgical site was swabbed with 70% ethanol, and a sagittal incision of the skin was made inferior to the occiput. Under the dissection microscope, the subcutaneous tissue and muscles (m. biventer cervicis and m. rectus capitis dorsalis major) were separated by blunt dissection with forceps. A pair of micro-retractors is used to hold the muscles apart. The dura mater was blotted dry with sterile cotton swab. CSF was collected using a capillary tube to penetrate into the cisterna magna through the dura mater, lateral to the arteria dorsalis spinalis, immediately frozen in liquid nitrogen and stored at −80° C.

Magnetic Resonance Imaging (MRI)

During the MRI scanning, mice were anesthetized with Isofluorane (5% for induction, 1-2% for maintenance) mixed with oxygen (1 liter/min) and delivered through a nasal mask. Once anesthetized, the animals were placed in a head-holder to assure reproducible positioning inside the magnet. Respiration rate was monitored and kept throughout the experimental period around 60-80 breaths per minute. MRI experiments were performed on 9.4 Tesla BioSpec Magnet 94/20 USR system (Bruker, Germany) equipped with gradient coil system capable of producing pulse gradient of up to 40 gauss/cm in each of the three directions. All MR images had been acquired with a receive quadrature mouse head surface coil and transmitter linear coil (Bruker). The T₂ maps were acquired using the multi-slice spin-echo (MSME) imaging sequence with the following parameters: a repetition delay (TR) of 3000 ms, 16-time echo (TE) increments (linearly from 10 to 160 ms), matrix dimension of 256×128 (interpolated to 256×256) and two averages, corresponding to an image acquisition time of 12 min 48 sec. The T2 dataset consisted of 16 images per slice. Thirteen continuous slices with slice thickness of 1.00 mm were acquired with a field of view (FOV) of 2.0×2.0 cm².

Image Analysis: A quantitative T₂ map was produced from multi-echo T₂-weighted images. The multi-echo signal was fitted to a mono-exponential decay to extract the T₂ value for each image pixel. All image analysis was performed using homemade scripts written in Matlab R2013B. Co-registration inter-subject and intra-subject was applied before the MRI dataset analysis. For optimal suitability to a mouse brain atlas (correction of head movements image artifacts), all images went through atlas registration: reslicing, realignment and smoothing, using the SPM software (version 12, UCL, London, UK). The results were reported as mean±SD. A t-test was used to compare means of two groups. A p value of less than 0.01 was considered statistically significant.

Histology

Sections from the spinal cord (C3-T6) were fixed in paraformaldehyde and embedded in paraffin for staining with luxol fast blue and cresyl echt violet. Subsequently, sections were examined by a blinded researcher and cresyl echt violet positive motor neurons in the ventral horn were counted to evaluate neuronal survival. Colon tissues were fixed in dry methanolic-Carnoy and stained with the nuclear stain Sytox green and the Muc2 mucin with the anti-MUC2C3 antiserum and goat anti-rabbit-Alexa 555 (Thermo Fisher Scientific)⁶⁶

Measuring Gut Epithelial Barrier Permeability by FITC-Dextran

On the day of the assay, 4 kDa fluorescein isothiocyanate (FITC)-dextran was dissolved in PBS to a concentration of 80 mg ml⁻¹. Mice were fasted for 4 hours prior to gavage with 150 μl dextran. Mice were anesthetized 3 hours following gavage and blood was collected and centrifuged at 1,000×g for 12 min at 4° C. Serum was collected and fluorescence was quantified at an excitation wavelength of 485 nm and 535 nm emission wavelength.

Flow Cytometry

WT and SOD1-Tg mice treated with Abx since 40 days of age or with water as controls were used for small-intestinal, colonic and spinal cord cellularity analysis either on day 140 (for small intestines and colons) or on days 60 and 140 (for spinal cords). Small intestinal and colonic samples were extensively washed from fecal matter followed by 2 mM EDTA dissociation in 37° C. for 30 min. Following extensive shaking, the epithelial fraction was discarded. Samples were then digested using DNAaseI and collagenase for lamina propria analysis. Spinal cord samples were harvested from individual mice, homogenized and incubated with a HBSS solution containing 2% BSA (Sigma-Aldrich), 1 mg/ml collagenase D (Roche), and 0.15 mg/ml DNasel, filtered through a 70 μm mesh. Homogenized sections were resuspended in 40% percoll, prior to density centrifugation (1000×g. 15 min at 20° C. with low acceleration and no brake. The isolated cells were washed with cold PBS and resuspended in PBS containing 1% BSA for direct cell surface staining. Single-cell suspensions were stained with antibodies for 45 min on ice against CD45, CD11b, CD11c, F4/80, Ly6C, Ly6G, B220, CD3, CD4, CD8 and NK1.1. Stained cells were analyzed on a BD-LSRFortessa cytometer and were analyzed with FlowJo software.

Mucus Proteomic Analysis

For proteome analyses isolated mucus samples were incubated overnight at 37° C. in reduction buffer (6M guanidinium hydrochloride, 0.1M Tris/HCl, pH 8.5, 5mM EDTA, 0.1 M DTT (Merck)) and soluble fraction was added on top of a spin-filter (10 kDa, PALL, Port Washington, N.Y.) for a filter-aided sample preparation following a previous protocol⁶⁷ where 6M GuHCl was used instead of urea. Proteins on the filters were alkylated and subsequently digested for 4 h with LysC (Wako, Richmond, Va.) followed by an overnight trypsin (Promega, Fitchburg, Wis.) digestion. Heavy peptides (SpikeTides TQL, JPT Peptide Technologies, Berlin, Germany) for Muc2 absolute quantification (10 peptides, 100 fmol each⁶⁸ were added before trypsin digestion. Peptides released from the filter after centrifugation were cleaned with StageTip C18 columns⁶⁹. NanoLC-MS/MS was performed on an EASY-nLC 1000 system (Thermo Fisher Scientific), connected to a Q Exactive HF Hybrid Quadrupole-Orbitrap Mass Spectrometer (Thermo Fisher Scientific) through a nanoelectrospray ion source. Peptides were separated with an in-house packed reverse-phase column (150×0.075 mm inner diameter, C18-AQ 3 μm) by a 30 min gradient from 10 to 45% of buffer B (A: 0.1% formic acid, B: 0.1% formic acid/80% acetonitrile) using a flow rate of 300 nl/min. Full mass spectra were acquired from 350-1,600 m/z with resolution of 60,000 (m/z 200). Up to 15 most intense peaks (charge state≥2) were fragmented and tandem mass spectra were acquired with a resolution of 15,000 and 20 s dynamic exclusion. For absolute quantification a separate targeted mass spectrometry method was used where only precursors and their fragments of the heavy and corresponding light peptides were scanned with a resolution of 30,000. Proteins were identified with the MaxQuant program (version 1.5.7.4⁷⁰) by searching against the mouse (downloaded 11.07.2018) UniProt protein database supplemented with an in-house database containing all the mouse mucin sequences (www(dot)medkem(dot)gu(dot)se/mucinbiology/databases/). Searches were performed with full tryptic specificity, maximum 2 missed cleavages, precursor tolerance of 20 ppm in the first search used for recalibration, followed by 7 ppm for the main search and 0.5 Da for fragment ions. Carbamido-methylation of cysteine was set to fixed modification and methionine oxidation and protein N-terminal acetylation were set as variable modification. The required false discovery rate (FDR) was set to 1% both for peptide and protein levels and the minimum required peptide length was set to six amino acids. Proteins were quantified based on MaxQuant label-free quantification (LFQ) option using a minimum of two peptides for quantification. Absolute quantification of Muc2 was performed with Skyline (version 4.2.0⁷¹).

Bacterial cultures: Akkermansia muciniphila (ATCC BAA-835), Akkermansia muciniphila (ATCC BAA-2869), Ruminococcus torques (ATCC 27756), Lactobacillus gasseri (ATCC 33323), Prevotella melaninogenica (ATCC 25845), Coprobacillus cateniformis (DSM-15921), Parabacteroides goldsteinii (DSM-19448), Lactobacillus murinus (DSM-100194), Parabacteroides distasonis (ATCC 8503), Eisenbergiella tayi (DSM-24404) Subdoligranulum variabile (SDM-15176) were grown in chopped meat medium (BD 297307) under anaerobic conditions (Coy Laboratory Products, 75% N₂, 20% CO2, 5% H₂) in 37° C. without shaking. Eggerthella lenta (DSM-15644) was grown in chopped meat medium supplemented with 0.5% arginine. All strains were validated for purity by whole-gene 16S sanger sequencing. WT and ΔnadA E. coli were originally obtained from the “Keio collection⁷²” and were grown on LB media (WT) or LB supplemented with 30 μg/ml kanamycin (ΔnadA). To measure bacterial in-vitro nicotinamide secretion, bacterial strains were grown in chopped meat medium until stationary phase, centrifuged and washed twice with M9 minimal medium with trace elements and glucose (4 g/l) and resuspended in M9 for 3 hrs under anaerobic conditions. Following centrifugation, 50 μl of the supernatant was collected for targeted nicotinamide measurements, and protein was extracted from the pellet using the BCA method: briefly: bacterial pellets were homogenized in RIPA buffer containing protease inhibitors, incubated for 45 min in 4° C. and centrifuged for 20 min, 14,000 r.p.m., at 4 ° C. Nicotinamide measurement in the media were then normalized to the total protein level in each sample.

Nucleic Acid Extraction

DNA purification: DNA was isolated from mouse fecal samples using PureLink™ Microbiome DNA Purification Kit (Invitrogen) according to manufacturer's recommendations.

DNA was isolated from patient stool swabs using PowerSoil DNA Isolation Kit (MOBIO Laboratories) optimized for an automated platform.

RNA Purification: Spinal cord, colon and muscle (Vastus lateralis) samples were harvested from mice and snap-frozen in liquid nitrogen. Tissues were homogenized in Tri Reagent (Sigma Aldrich). RNA was purified using standard chloroform extraction. Two micrograms of total RNA were used to generate cDNA (HighCapacity cDNA Reverse Transcription kit; Applied Biosystems).

PCR was performed using Kapa Sybr qPCR kit (Kapa Biosystems) on a Viia7 instrument (Applied Biosystems). PCR conditions were 95° C. for 20 s, followed by 40 cycles of 95° C. for 3 s and 60° C. for 30 s. Data were analyzed using the AACt method with 16S serving as the reference housekeeping gene. 16S cycles were assured to be insensitive to the experimental conditions.

Nucleic Acid Processing and Library Preparation

16S qPCR Protocol for Quantification of Bacterial DNA: DNA templates were diluted to 1 ng/ul before amplifications with the primer sets (indicated in Table 1) using the Fast Sybr™ Green Master Mix (ThermoFisher) in duplicates. Amplification conditions for Akkermansia muciniphila were: Denaturation 95° C. for 3 minutes, followed by 40 cycles of Denaturation 95° C. for 3 seconds; annealing 66° C. for 30 seconds followed by meting curve. Amplification conditions for total bacteria (16S rRNA) were: Denaturation 95° C. for 3 minutes, followed by 40 cycles of Denaturation 95° C. for 3 seconds; annealing 60° C. for 30 seconds followed by meting curve. Duplicates with >2 cycle difference were excluded from analysis. The CT value for any sample not amplified after 40 cycles was defined as 40 (threshold of detection).

TABLE 1
Primers used in qPCR analysis.

| Primer | | |
|---|---|---|
| ID | Sequence | Target & reference |
| AM1 | CAGCACGTGAAGGTGGGGAC | Akkermansia muciniphila |
| | (SEQ ID NO: 1) | 16S rRNA gene |
| AM2 | CCTTGCGGTTGGCTTCAGAT | Akkermansia muciniphila |
| | (SEQ ID NO: 2) | 16S rRNA gene |
| F-S | CAACGCGMARAACCTTACC | 16S rRNA gene |
| | (SEQ ID NO: 3) | |
| F-AQ | CTAACCGANGAACCTYACC | 16S rRNA gene |
| | (SEQ ID NO: 4) | |
| F-UC3 | ATACGCGARGAACCTTACC | 16S rRNA gene |
| | (SEQ ID NO: 5) | |
| F-PP | CNACGCGAAGAACCTTANC | 16S rRNA gene |
| | (SEQ ID NO: 6) | |
| R-S | CGACRRCCATGCANCACCT | 16S rRNA gene |
| | (SEQ ID NO: 7) | |

16S rDNA Sequencing

For 16S amplicon pyrosequencing, PCR amplification was performed spanning the V4 region using the primers 515F/806R of the 16S rRNA gene and subsequently sequenced using 2×250 bp paired-end sequencing (Illumina MiSeq). Custom primers were added to Illumina MiSeq kit resulting in 253 bp fragment sequenced following paired end joining to a depth of 110,998±66,946 reads (mean±SD).

Read1:
(SEQ ID NO: 8)
TATGGTAATTGTGTGCCAGCMGCCGCGGTAA
Read2:
(SEQ ID NO: 9)
AGTCAGTCAGCCGGACTACHVGGGTWTCTAAT
Index sequence primer:
(SEQ ID NO: 10)
ATTAGAWACCCBDGTAGTCCGGCTGACTGACTATTAGAA

Whole Genome Shotgun Sequencing

100 ng of purified DNA was sheared with a Covaris E220X sonicator. Illumina compatible libraries were prepared as described (Suez et al., 2014), and sequenced on the Illumina NextSeq platform with a read length of 80 bp to a depth of 10M reads for human samples, 1M reads for AM treated mice samples and 5M reads for the comparison between naïve WT and SOD1-Tg mice.

RNA-Seq

Ribosomal RNA was selectively depleted by RnaseH (New England Biolabs, M0297) according to a modified version of a published method (Adiconis et al., 2013). Specifically, a pool of 50 bp DNA oligos (25 nM, IDT, indicated in Table 3) that is complementary to murine rRNA18S and 28S, was resuspended in 75 μl of 10 mM Tris pH 8.0. Total RNA (100-1000 ng in 10 μl H₂O) were mixed with an equal amount of rRNA oligo pool, diluted to 2 μl and 3 μl 5× rRNA hybridization buffer (0.5 M Tris-HCl, 1 M NaCl, titrated with HCl to pH 7.4) was added. Samples were incubated at 95° C. for 2 minutes, then the temperature was slowly decreased (−0.1° C./s) to 37° C. RNAseH enzyme mix (2 μl of 10U RNAseH, 2 μl 10× RNAseH buffer, 1 μl H2O, total 5 μl mix) was prepared 5 minutes before the end of the hybridization and preheated to 37° C. The enzyme mix was added to the samples when they reached 37° C. and they were incubated at this temperature for 30 minutes. Samples were purified with 2.2× SPRI beads (Ampure XP, Beckmann Coulter) according to the manufacturers' instructions. Residual oligos were removed with DNAse treatment (ThermoFisher Scientific, AM2238) by incubation with 5 μl DNAse reaction mix (1 μl Trubo DNAse, 2.5 μl Turbo DNAse 10×buffer, 1.5 μl H2O) that was incubated at 37° C. for 30 minutes.

Samples were again purified with 2.2× SPRI beads and suspended in 3.6 pi priming mix (0.3 μl random primers of New England Biolab, E7420, 3.3 pi H₂O). Samples were subsequently primed at 65° C. for 5 minutes. Samples were then transferred to ice and 2 μl of the first strand mix was added (1 μl 5×first strand buffer, NEB E7420; 0.125 μl RNAse inhibitor, NEB E7420; 0.25 μl ProtoScript II reverse transcriptase, NEB E7420; and 0.625 μl of 0.2 μl/ml Actinomycin D, Sigma, A1410). The first strand synthesis and all subsequent library preparation steps were performed using NEBNext Ultra Directional RNA Library Prep Kit for Illumina (NEB, E7420) according to the manufacturers' instructions (all reaction volumes reduced to a quarter).

TABLE 3
DNA oligos used for rRNA depletion

| Oligo name | Sequence |
|---|---|
| AG9327_18_1 | TAATGATCCTTCCGCAGGTTCACCTACGGAAACCTTGTTACGACTTT |
| | TAC |
| | (SEQ ID NO: 11) |
| AG9328_18_2 | TTCCTCTAGATAGTCAAGTTCGACCGTCTTCTCAGCGCTCCGCCAGG |
| | GCC |
| | (SEQ ID NO: 12) |
| AG9329_18_3 | GTGGGCCGACCCCGGCGGGGCCGATCCGAGGGCCTCACTAAACCAT |
| | CCAA |
| | (SEQ ID NO: 13) |
| AG9330_18_4 | TCGGTAGTAGCGACGGGCGGTGTGTACAAAGGGCAGGGACTTAATC |
| | AACG |
| | (SEQ ID NO: 14) |
| AG9331_18_5 | CAAGCTTATGACCCGCACTTACTCGGGAATTCCCTCGTTCATGGGGA |
| | ATA |
| | (SEQ ID NO: 15) |
| AG9332_18_6 | ATTGCAATCCCCGATCCCCATCACGAATGGGGTTCAACGGGTTACCC |
| | GCG |
| | (SEQ ID NO: 16) |
| AG9333_18_7 | CCTGCCGGCGTAGGGTAGGCACACGCTGAGCCAGTCAGTGTAGCGC |
| | GCGT |
| | (SEQ ID NO: 17) |
| AG9334_18_8 | GCAGCCCCGGACATCTAAGGGCATCACAGACCTGTTATTGCTCAATC |
| | TCG |
| | (SEQ ID NO: 18) |
| AG9335_18_9 | GGTGGCTGAACGCCACTTGTCCCTCTAAGAAGTTGGGGGACGCCGA |
| | CCGC |
| | (SEQ ID NO: 19) |
| AG9336_18_10 | TCGGGGGTCGCGTAACTAGTTAGCATGCCAGAGTCTCGTTCGTTATC |
| | GGA |
| | (SEQ ID NO: 20) |
| AG9337_18_11 | ATTAACCAGACAAATCGCTCCACCAACTAAGAACGGCCATGCACCA |
| | CCAC |
| | (SEQ ID NO: 21) |
| AG9338_18_12 | CCACGGAATCGAGAAAGAGCTATCAATCTGTCAATCCTGTCCGTGTC |
| | CGG |
| | (SEQ ID NO: 22) |
| AG9339_18_13 | GCCGGGTGAGGTTTCCCGTGTTGAGTCAAATTAAGCCGCAGGCTCC |
| | ACTC |
| | (SEQ ID NO: 23) |
| AG9340_18_14 | CTGGTGGTGCCCTTCCGTCAATTCCTTTAAGTTTCAGCTTTGCAACCA |
| | TA |
| | (SEQ ID NO: 24) |
| AG9341_18_15 | CTCCCCCCGGAACCCAAAGACTTTGGTTTCCCGGAAGCTGCCCGGCG |
| | GGT |
| | (SEQ ID NO: 25) |
| AG9342_18_16 | CATGGGAATAACGCCGCCGCATCGCCGGTCGGCATCGTTTATGGTC |
| | GGAA |
| | (SEQ ID NO: 26) |
| AG9343_18_17 | CTACGACGGTATCTGATCGTCTTCGAACCTCCGACTTTCGTTCTTGAT |
| | TA |
| | (SEQ ID NO: 27) |
| AG9344_18_18 | ATGAAAACATTCTTGGCAAATGCTTTCGCTCTGGTCCGTCTTGCGCC |
| | GGT |
| | (SEQ ID NO: 28) |
| AG9345_18_19 | CCAAGAATTTCACCTCTAGCGGCGCAATACGAATGCCCCCGGCCGT |
| | CCCT |
| | (SEQ ID NO: 29) |
| AG9346_18_20 | CTTAATCATGGCCTCAGTTCCGAAAACCAACAAAATAGAACCGCGG |
| | TCCT |
| | (SEQ ID NO: 30) |
| AG9347_18_21 | ATTCCATTATTCCTAGCTGCGGTATCCAGGCGGCTCGGGCCTGCTTT |
| | GAA |
| | (SEQ ID NO: 31) |
| AG9348_18_22 | CACTCTAATTTTTTCAAAGTAAACGCTTCGGGCCCCGCGGGACACTC |
| | AGC |
| | (SEQ ID NO: 32) |
| AG9349_18_23 | TAAGAGCATCGAGGGGGCGCCGAGAGGCAAGGGGCGGGGACGGGC |
| | GGTGG |
| | (SEQ ID NO: 33) |
| AG9350_18_24 | CTCGCCTCGCGGCGGACCGCCCGCCCGCTCCCAAGATCCAACTACG |
| | AGCT |
| | (SEQ ID NO: 34) |
| AG9351_18_25 | TTTTAACTGCAGCAACTTTAATATACGCTATTGGAGCTGGAATTACC |
| | GCG |
| | (SEQ ID NO: 35) |
| AG9352_18_26 | GCTGCTGGCACCAGACTTGCCCTCCAATGGATCCTCGTTAAAGGATT |
| | TAA |
| | (SEQ ID NO: 36) |
| AG9353_18_27 | AGTGGACTCATTCCAATTACAGGGCCTCGAAAGAGTCCTGTATTGTT |
| | ATT |
| | (SEQ ID NO: 37) |
| AG9354_18_28 | TTTCGTCACTACCTCCCCGGGTCGGGAGTGGGTAATTTGCGCGCCTG |
| | CTG |
| | (SEQ ID NO: 38) |
| AG9355_18_29 | CCTTCCTTGGATGTGGTAGCCGTTTCTCAGGCTCCCTCTCCGGAATC |
| | GAA |
| | (SEQ ID NO: 39) |
| AG9356_18_30 | CCCTGATTCCCCGTCACCCGTGGTCACCATGGTAGGCACGGCGACTA |
| | CCA |
| | (SEQ ID NO: 40) |
| AG9357_18_31 | TCGAAAGTTGATAGGGCAGACGTTCGAATGGGTCGTCGCCGCCACG |
| | GG |
| | (SEQ ID NO: 41) |
| AG9358_18_32 | GCGTGCGATCGGCCCGAGGTTATCTAGAGTCACCAAAGCCGCCGGC |
| | GCCC |
| | (SEQ ID NO: 42) |
| AG9359_18_33 | GCCCCCCGGCCGGGGCCGGAGAGGGGCTGACCGGGTTGGTTTTGAT |
| | CTGA |
| | (SEQ ID NO: 43) |
| AG9360_18_34 | TAAATGCACGCATCCCCCCCGCGAAGGGGGTCAGCGCCCGTCGGCA |
| | TGTA |
| | (SEQ ID NO: 44) |
| AG9361_18_35 | TTAGCTCTAGAATTACCACAGTTATCCAAGTAGGAGAGGAGCGAGC |
| | GACC |
| | (SEQ ID NO: 45) |
| AG9362_18_36 | AAAGGAACCATAACTGATTTAATGAGCCATTCGCAGTTTCACTGTAC |
| | CGG |
| | (SEQ ID NO: 46) |
| AG9363_18_37 | CCGTGCGTACTTAGACATGCATGGCTTAATCTTTGAGACAAGCATAT |
| | GCT |
| | (SEQ ID NO: 47) |
| AG9364_18_38 | TGGCTTAATCTTTGAGACAAGCATATGCTACTGGCAGGATCAACCA |
| | GGTA |
| | (SEQ ID NO: 48) |
| AG9466_5.8_1 | AAGCGACGCTCAGACAGGCGTAGCCCCGGGAGGAACCCGGGGCCG |
| | CAAGT |
| | (SEQ ID NO: 49) |
| AG9467_5.8_2 | GCGTTCGAAGTGTCGATGATCAATGTGTCCTGCAATTCACATTAATT |
| | CTC |
| | (SEQ ID NO: 50) |
| AG9468_5.8_3 | GCAGCTAGCTGCGTTCTTCATCGACGCACGAGCCGAGTGATCCACC |
| | GCTA |
| | (SEQ ID NO: 51) |
| AG9469_16_1 | AAACCCTGTTCTTGGGTGGGTGTGGGTATAATACTAAGTTGAGATGA |
| | TAT |
| | (SEQ ID NO: 52) |
| AG9470_16_2 | CATTTACGGGGGAAGGCGCTTTGTGAAGTAGGCCTTATTTCTCTTGT |
| | CCT |
| | (SEQ ID NO: 53) |
| AG9471_16_3 | TTCGTACAGGGAGGAATTTGAANGTAGATAGAAACCGACCTGGATT |
| | ACTC |
| | (SEQ ID NO: 54) |
| AG9472_16_4 | CGGTCTGAACTCAGATCACGTAGGACTTTAATCGTTGAACAAACGA |
| | ACCT |
| | (SEQ ID NO: 55) |
| AG9473_16_5 | TTAATAGCGGCTGCACCATCGGGATGTCCTGATCCAACATCGAGGTC |
| | GTA |
| | (SEQ ID NO: 56) |
| AG9474_16_6 | AACCCTATTGTTGATATGGACTCTAGAATAGGATTGCGCTGTTATCC |
| | CTA |
| | (SEQ ID NO: 57) |
| AG9475_16_7 | GGGTAACTTGTTCCGTTGGTCAAGTTATTGGATCAATTGAGTATAGT |
| | AGT |
| | (SEQ ID NO: 58) |
| AG9476_16_8 | TCGCTTTGACTGGTGAAGTCTTAGCATGTACTGCTCGGAGGTTGGGT |
| | TCT |
| | (SEQ ID NO: 59) |
| AG9477_16_9 | GCTCCGAGGTCGCCCCAACCGAAATTTTTAATGCAGGTTTGGTAGTT |
| | TAG |
| | (SEQ ID NO: 60) |
| AG9478_16_10 | GACCTGTGGGTTTGTTAGGTACTGTTTGCATTAATAAATTAAAGCTC |
| | CAT |
| | (SEQ ID NO: 61) |
| AG9479_16_11 | AGGGTCTTCTCGTCTTGCTGTGTTATGCCCGCCTCTTCACGGGCAGG |
| | TCA |
| | (SEQ ID NO: 62) |
| AG9480_16_12 | ATTTCACTGGTTAAAAGTAAGAGACAGCTGAACCCTCGTGGAGCCA |
| | TTCA |
| | (SEQ ID NO: 63) |
| AG9481_16_13 | TACAGGTCCCTATTTAAGGAACAAGTGATTATGCTACCTTTGCACGG |
| | TTA |
| | (SEQ ID NO: 64) |
| AG9482_16_14 | GGGTACCGCGGCCGTTAAACATGTGTCACTGGGCAGGCGGTGCCTC |
| | TAAT |
| | (SEQ ID NO: 65) |
| AG9483_16_15 | ACTGGTGATGCTAGAGGTGATGTTTTTGGTAAACAGGCGGGGTAAG |
| | ATTT |
| | (SEQ ID NO: 66) |
| AG9484_16_16 | GCCGAGTTCCTTTTACTTTTTTTAACCTTTCCTTATGAGCATGCCTGT |
| | GT |
| | (SEQ ID NO: 67) |
| AG9485_16_17 | TGGGTTGACAGTGAGGGTAATAATGACTTGTTGGTTGATTGTAGATA |
| | TTG |
| | (SEQ ID NO: 68) |
| AG9486_16_18 | GGCTGTTAATTGTCAGTTCAGTGTTTTAATCTGACGCAGGCTTATGC |
| | GGA |
| | (SEQ ID NO: 69) |
| AG9487_16_19 | GGAGAATGTTTTCATGTTACTTATACTAACATTAGTTCTTCTATAGG |
| | GTG |
| | (SEQ ID NO: 70) |
| AG9488_16_20 | ATAGATTGGTCCAATTGGGTGTGAGGAGTTCAGTTATATGTTTGGGA |
| | TTT |
| | (SEQ ID NO: 71) |
| AG9489_16_21 | TTTAGGTAGTGGGTGTTGAGCTTGAACGCTTTCTTAATTGGTGGCTG |
| | CTT |
| | (SEQ ID NO: 72) |
| AG9490_16_22 | TTAGGCCTACTATGGGTGTTAAATTTTTTACTCTCTCTACAAGGTTTT |
| | TT |
| | (SEQ ID NO: 73) |
| AG9491_16_23 | CCTAGTGTCCAAAGAGCTGTTCCTCTTTGGACTAACAGTTAAATTTA |
| | CAA |
| | (SEQ ID NO: 74) |
| AG9492_16_24 | GGGATTTAGAGGGTTCTGTGGGCAAATTTAAAGTTGAACTAAGATT |
| | CTA |
| | (SEQ ID NO: 75) |
| AG9493_16_25 | TCTTGGACAACCAGCTATCACCAGGCTCGGTAGGTTTGTCGCCTCTA |
| | CCT |
| | (SEQ ID NO: 76) |
| AG9494_16_26 | ATAAATCTTCCCACTATTTTGCTACATAGACGGGTGTGCTCTTTTAG |
| | CTG |
| | (SEQ ID NO: 77) |
| AG9495_16_27 | TTCTTAGGTAGCTCGTCTGGTTTCGGGGGTCTTAGCTTTGGCTCTCCT |
| | TG |
| | (SEQ ID NO: 78) |
| AG9496_16_28 | CAAAGTTATTTCTAGTTAATTCATTATGCAGAAGGTATAGGGGTTAG |
| | TCC |
| | (SEQ ID NO: 79) |
| AG9497_16_29 | TTGCTATATTATGCTTGGTTATAATTTTTCATCTTTCCCTTGCGGTAC |
| | TA |
| | (SEQ ID NO: 80) |
| AG9498_16_30 | TATCTATTGCGCCAGGTTTCAATTTCTATCGCCTATACTTTATTTGGG |
| | TA |
| | (SEQ ID NO: 81) |
| AG9499_16_31 | AATGGTTTGGCTAAGGTTGTCTGGTAGTAAGGTGGAGTGGGTTTGG |
| | GGCT |
| | (SEQ ID NO: 82) |
| AG9500_12_1 | GTTCGTCCAAGTGCACTTTCCAGTACACTTACCATGTTACGACTTGT |
| | CTC |
| | (SEQ ID NO: 83) |
| AG9501_12_2 | CTCTATATAAATGCGTAGGGGTTTTAGTTAAATGTCCTTTGAAGTAT |
| | ACT |
| | (SEQ ID NO: 84) |
| AG9502_12_3 | TGAGGAGGGTGACGGGCGGTGTGTACGCGCTTCAGGGCCCTGTTCA |
| | ACTA |
| | (SEQ ID NO: 85) |
| AG9503_12_4 | AGCACTCTACTCTTAGTTTACTGCTAAATCCACCTTCGACCCTTAAG |
| | TTT |
| | (SEQ ID NO: 86) |
| AG9504_12_5 | CATAAGGGCTATCGTAGTTTTCTGGGGTAGAAAATGTAGCCCATTTC |
| | TTG |
| | (SEQ ID NO: 87) |
| AG9505_12_6 | CCACCTCATGGGCTACACCTTGACCTAACGTCTTTACGTGGGTACTT |
| | GCG |
| | (SEQ ID NO: 88) |
| AG9506_12_7 | CTTACTTTGTAGCCTTCATCAGGGTTTGCTGAAGATGGCGGTATATA |
| | GGC |
| | (SEQ ID NO: 89) |
| AG9507_12_8 | TGAGCAAGAGGTGGTGAGGTTGATCGGGGTTTATCGATTACAGAAC |
| | AGGC |
| | (SEQ ID NO: 90) |
| AG9508_12_9 | TCCTCTAGAGGGATATGAAGCACCGCCAGGTCCTTTGAGTTTTAAGC |
| | TGT |
| | (SEQ ID NO: 91) |
| AG9509_12_10 | GGCTCGTAGTGTTCTGGCGAGCAGTTTTGTTGATTTAACTGTTGAGG |
| | TTT |
| | (SEQ ID NO: 92) |
| AG9510_12_11 | AGGGCTAAGCATAGTGGGGTATCTAATCCCAGTTTGGGTCTTAGCTA |
| | TTG |
| | (SEQ ID NO: 93) |
| AG9511_12_12 | TGTGTTCAGATATGTTAAAGCCACTTTCGTAGTCTATTTTGTGTCAAC |
| | TG |
| | (SEQ ID NO: 94) |
| AG9512_12_13 | GAGTTTTTTACAACTCAGGTGAGTTTTAGCTTTATTGGGGAGGGGGT |
| | GAT |
| | (SEQ ID NO: 95) |
| AG9513_12_14 | CTAAAACACTCTTTACGCCGGCTTCTATTGACTTGGGTTAATCGTGT |
| | GAC |
| | (SEQ ID NO: 96) |
| AG9514_12_15 | CGCGGTGGCTGGCACGAAATTGACCAACCCTGGGGTTAGTATAGCT |
| | TAGT |
| | (SEQ ID NO: 97) |
| AG9515_12_16 | TAAACTTTCGTTTATTGCTAAAGGTTAATCACTGCTGTTTCCCGTGG |
| | G |
| | (SEQ ID NO: 98) |
| AG9516_12_17 | TGTGGCTAGGCTAAGCGTTTTGAGCTGCATTGCTGCGTGCTTGATGC |
| | TTG |
| | (SEQ ID NO: 99) |
| AG9517_12_18 | TTCCTTTTGATCGTGGTGATTTAGAGGGTGAACTCACTGGAACGGGG |
| | ATG |
| | (SEQ ID NO: 100) |
| AG9518_12_19 | CTTGCATGTGTAATCTTACTAAGAGCTAATAGAAAGGCTAGGACCA |
| | AACC |
| | (SEQ ID NO: 101) |
| AG9519_5_1 | AAAGCCTACAGCACCCGGTATTCCCAGGCGGTCTCCCATCCAAGTA |
| | CTAA |
| | (SEQ ID NO: 102) |
| AG9520_5_2 | CCAGGCCCGACCCTGCTTAGCTTCCGAGATCAGACGAGATCGGGCG |
| | CGTT |
| | (SEQ ID NO: 103) |
| AG9521_5_3 | TTCCGAGATCAGACGAGATCGGGCGCGTTCAGGGTGGTATGGCCGT |
| | AGAC |
| | (SEQ ID NO: 104) |

16S rDNA Analysis

Overlapping paired-end FASTQ files were matched and processed in a data curation pipeline implemented in Qiime 2 version 2018.4.0 (Qiime2) (Caporaso et al., 2010). Paired-end sequence data were demultiplexed according to sample specific barcodes using Qiime2 demux-emp-paired. Trimming and amplicon sequence variant (ASV) picking were carried out with the use of DADA2 (Callahan et al., 2016). Alpha rarefaction curves were plotted using Qiime2 alpha-rarefaction and were used to set an appropriate subsampling depth for each comparison. Samples were rarefied using Qiime2 feature-table rarefy (Weiss et al., 2017). Samples with a read depth lower than the relevant subsampling depth were excluded from the analysis. ASV's were assigned with taxonomic annotations using a Naïve-Bayes fitted classifier trained on August 2013, 97% identity Greengenes rRNA database (McDonald et al., 2012). Relative abundance tables were calculated using Qiime2 feature-table summarize-taxa. Ordination plots were calculated from Unweighted and Weighted UniFrac distance matrix using principal coordinates analysis (PCoA).

Metagenomic Analysis

For metagenome analysis, metagenomic reads containing IIlumina adapters and low-quality reads were filtered and low-quality read edges were trimmed. Host DNA was detected by mapping with GEM (Marco-Sola et al., 2012) to the human or mouse genome (hg19 or mm10 respectively) with inclusive parameters, and host reads were removed. For mice metagenomes 1 million reads were subsampled and for humans 7-10 million reads. Relative abundances from metagenomic sequencing were computed using MetaPhlAn2 (Loh et al., 2016) with default parameters. MetaPhlAn relative abundances were capped at a level of 5×10⁻⁴. KO relative abundance was obtained by mapping to KEGG (Kanehisa et al., 2006) bacterial genes database using DIAMOND (Buchfink et al., 2015), considering only the first hit, and allowing e-value<0.0001. The relative abundance of a KO was determined as the sum of all reads mapped to bacterial genes associated with that KO, divided by the total number of mapped reads in a sample. KO relative abundances were capped at a level of 2×10⁻⁵ for mice and 2×10⁻⁷ for humans. Taxa and KOs present in less than 10% of samples were discarded.

Metabolites selection: Using the top 12 significant serum metabolites altered by Abx in WT and SOD1-Tg mice, we first downloaded all nucleotide sequences of KEGG genes with potential to synthesize or degrade the 12 metabolites. Next we built a bowtie index of KEGG genes and mapped to it SOD1-Tg and WT metagenome samples. Finally, we obtained all mapped reads and for every sample and KEGG gene, we report the number of reads mapped to the KEGG gene and its mean score. Scores are as defined by bowtie2⁸⁴ and range between 0 to −45, where 0 denotes perfect match.

RNAseq Analysis

Data pre-processing: bcl files were converted to fastq and adaptor trimming was performed using bcl2fastq. Then, reads were aligned to the mm10 reference genome (UCSC) using STAR (splice site aware alignment). Secondary alignments and PCR/optical duplicates were removed using samtools view -h -F 256-F 1024. Alignments were binned to genes using htseq-count count (htseq-count -a 5-s reverse -r). Transcript integrity number (TIN) medians were calculated using RSeQC. (tin.py.bed file: mm10 RefSeq.bed.gz downloaded from sourceforgedotnet/projects/rseqc/files/BED/Mouse_Mus_musculus/)

Differential gene expression: For each comparison, genes with reads≥10⁻⁴ out of total reads and expressed in at least fifth of a group in each comparison were included in the analysis. Deseq2 models were fitted for each comparison separately [design: counts˜group+median (TIN)]. Differentially expressed genes were found using Wald-test on Deseq2 objects. Heatmaps were created using the regularized log transformed data (rlog).

Gene set enrichment analysis: For each gene, we calculated the following score out of its DESeq results: −log(padj} sign(log2FoldChange). bulk.gsea function was used from liger package, with the www(dot)ge-lab(dot)org/gskb/2-MousePath/MousePath_GO_gmtdotgmt as the universe model.

Non-Targeted Metabolomics

Sera and cecal samples were collected, immediately frozen in liquid nitrogen and stored at −80° C. Sample preparation and analysis was performed by Metabolon Inc. Samples were prepared using the automated MicroLab STAR system (Hamilton). To remove protein, dissociated small molecules bound to protein or trapped in the precipitated protein matrix, and to recover chemically diverse metabolites, proteins were precipitated with methanol. The resulted extract was divided into five fractions: one for analysis by UPLC-MS/MS with negative ion mode electrospray ionization, one for analysis by UPLC-MS/MS with positive ion mode electrospray ionization, one for LC polar platform, one for analysis by GC-MS and one sample was reserved for backup. Samples were placed briefly on a TurboVap (Zymark) to remove the organic solvent. For LC, the samples were stored overnight under nitrogen before preparation for analysis. For GC, each sample was dried under vaccum overnight before preparation for analysis.

Data extraction and compound identification: Raw data was extracted, peak-identified and QC processed using Metabolon's hardware and software. Compound were identified by comparison to library entries of purified standards or recurrent unknown entities.

Metabolite quantification and data normalization: Peaks were quantified using area-under-the-curve. For studies spanning multiple days, a data normalization step was performed to correct variation resulting from instrument inter-day tuning differences.

Targeted Metabolomics

50 ng/ml of D5-Glutamic acid and 50 ng/ml of D4-Nicotinamide (Cambridge Isotope Laboratories) were added to all samples as internal standards. The samples (in 50% Methanol) were dried in a speed vac to blow off the methanol before drying to completion in a lyophilizer. All samples were re-dissolved in 100 μl of 0.1% formic acid.

Liquid Chromatography: Liquid chromatography was performed on a Waters Acquity UPLC system. Metabolites were separated on an Acquity HSS T3 column (2.1×150 mm, 1.8 μm particle size; Waters) at 40° C. using a 10-min program. Mobile phase consisted of (A) water and (B) acetonitrile each containing 0.1% formic acid. Gradient conditions were: 0 to 1 min=99.9% A 0.1% B; 1 to 6 min=0.1% to 10.0% B; 6 to 7 min=10% to 100% B; 7.0 to 7.2 min=100% B; 7.2 to 10 min=99.9% A, 0.1% B. Injection volume was 1.0 μl, and flow rate was 0.3 ml/min.

Mass Spectrometry: LC-MS/MS analysis was performed on a Waters Xevo triple quadrupole equipped with a Zspray ESI source. MRM was performed in the positive ion mode. Other MS parameters included: desolvation temperature at 600° C., desolvation gas flow at 900 L/Hr, cone gas flow at 150 L/Hr nebulizer pressure at 7 Bar, capillary voltage (CV) at 2.53 kV. The MRM transitions used were: (a) Glutamic acid: 148.1>84.1 and 148.1>102, collision energy (CE) 15 and 11 V respectively. (b) L-D5-Glutamic acid: 153.1>88.1 and 153>107, CE 15 and 11 V 15 respectively. (c) Nicotinamide: 123>78 and 123>80, CE 19, 13 V respectively and (d) D4-Nicotinamide 127>81 and 127>84, CE 19, 17 V respectively. Argon (0.10 mg/min) was used as collision gas. TargetLynx (Waters) was used for Qualitative and Quantitative analysis.

Patients and Control Individuals

Clinical trial: The human trial was approved by the Hadassah Medical Center Institutional Review Board (IRB approval numbers HMO-16-0396) and Weizmann Institute of Science Bioethics and Embryonic Stem Cell Research oversight committee (IRB approval numbers 365-1). Written informed consent was obtained from all subjects.

Exclusion and inclusion criteria (human cohorts): All subjects fulfilled the following inclusion criteria: males and females, aged 18-70, who are currently not following any diet regime 25 or dietitian consultation and are able to provide informed consent. Exclusion criteria included: (i) pregnancy or fertility treatments; (ii) usage of antibiotics or antifungals within three months prior to participation; (iii) consumption of probiotics in any form within one month prior to participation, (iv) chronically active inflammatory or neoplastic disease in the three years prior to enrollment; (v) chronic gastrointestinal disorder, including inflammatory bowel disease and celiac disease; (vi) myocardial infarction or cerebrovascular accident in the 6 months prior to participation; (vii) coagulation disorders; (viii) chronic immunosuppressive medication usage; (ix) pre-diagnosed type I or type II diabetes mellitus or treatment with anti-diabetic medication. Adherence to inclusion and exclusion criteria was validated by medical doctors.

TABLE 4
Participant details

| #Participant | Sex | Group | Age (y) | Weight (kg) | Height (cm) | ALS FRS | Relation |
|---|---|---|---|---|---|---|---|
| ALS_728 | F | ALS | 33 | 39.3 | 170 | 19 | |
| ALS_747 | M | ALS | 61 | 84.5 | 181 | 28 | |
| ALS_1890 | M | ALS | 67 | 83.5 | 171 | 42 | |
| ALS_1447 | M | ALS | 68 | 80.2 | 170 | 37 | |
| ALS_1633 | M | ALS | 40 | 69.4 | 175 | 25 | |
| ALS_1640 | M | ALS | 76 | 92 | 170 | 39 | |
| ALS_1641 | M | ALS | 51 | 68.3 | 181 | 27 | |
| ALS_1659 | F | ALS | 55 | 48.7 | 165 | 8 | |
| ALS_1671 | F | ALS | 55 | 51.3 | 163 | 28 | |
| ALS_1680 | F | ALS | 53 | 55.7 | 170 | 38 | |
| ALS_1717 | M | ALS | 39 | 69.9 | 173 | 29 | |
| ALS_1730 | F | ALS | 70 | 54.9 | 150 | 19 | |
| ALS_1731 | M | ALS | 68 | 102.3 | 178 | 42 | |
| ALS_1739 | M | ALS | 61 | 66.4 | 165 | 26 | |
| ALS_1745 | M | ALS | 47 | 64 | 175 | 24 | |
| ALS_1753 | F | ALS | 72 | | 156 | 18 | |
| ALS_1764 | M | ALS | 49 | 79.5 | 180 | 37 | |
| ALS_1781 | M | ALS | 53 | 83.2 | 176 | 39 | |
| ALS_1784 | F | ALS | 51 | 73.5 | 165 | 28 | |
| ALS_1787 | M | ALS | 55 | 63.5 | 171 | 32 | |
| ALS_1789 | M | ALS | 60 | 80.2 | 160 | 38 | |
| ALS_1799 | M | ALS | 57 | 78 | 167 | 40 | |
| ALS_1814 | M | ALS | 59 | 64.3 | 178 | 43 | |
| ALS_1841 | M | ALS | 47 | 69.7 | 186 | 37 | |
| ALS_1779 | M | ALS | 58 | 86.2 | 179 | 38 | |
| ALS_1825 | M | ALS | 56 | 100 | 174 | 22 | |
| ALS_1851 | M | ALS | 65 | 67 | 174 | 38 | |
| ALS_1869 | M | ALS | 64 | 62.4 | 160 | 38 | |
| ALS_1883 | M | ALS | 71 | | 180 | 27 | |
| ALS_1857 | F | ALS | 75 | 72.7 | 154 | 41 | |
| ALS_1823 | M | ALS | 67 | 74 | 176 | 36 | |
| ALS_1888 | M | ALS | 57 | 89.8 | 187 | 42 | |
| C_728 | F | Healthy | 46 | 73 | 160 | 48 | Wife |
| C_747 | F | Healthy | 56 | 65 | 163 | 48 | Wife |
| C_1742 | F | Healthy | 43 | 75 | 164 | 48 | Mother |
| C_1633 | F | Healthy | 42 | 80 | 167 | 48 | Wife |
| C_1640 | F | Healthy | 72 | 54 | 158 | 48 | Wife |
| C_1641 | M | Healthy | | | | 48 | Husband |
| C_1659 | M | Healthy | 58 | 88 | 187 | 48 | Husband |
| C_1671 | M | Healthy | 55 | 99 | 185 | 48 | Husband |
| C_1680 | M | Healthy | 59 | 87 | 193 | 48 | Husband |
| C_1717 | F | Healthy | 46 | 75 | 155 | 48 | Wife |
| C_1730 | M | Healthy | 74 | 80 | 176 | 48 | Husband |
| C_1731 | F | Healthy | 67 | 50 | 162 | 48 | Wife |
| C_1739 | F | Healthy | 59 | 71 | 141 | 48 | Wife |
| C_1745 | F | Healthy | 46 | 65 | 175 | 48 | Wife |
| C_1753 | M | Healthy | 50 | 70 | 172 | 48 | Husband |
| C_1764 | F | Healthy | 44 | 85 | 163 | 48 | Wife |
| C_1781 | F | Healthy | 50 | 90 | 160 | 48 | Wife |
| C_1784 | M | Healthy | 50 | 104 | 174 | 48 | Husband |
| C_1799 | F | Healthy | 56 | 65 | 165 | 48 | Wife |
| C_1814 | F | Healthy | 55 | 71 | 163 | 48 | Wife |
| C_1851 | F | Healthy | 66 | 68.4 | 167 | 48 | Wife |
| C_1833 | F | Healthy | | | | 48 | Wife |
| C_1857 | M | Healthy | | | | 48 | Husband |
| C_1888 | F | Healthy | 59.5 | 78 | 160 | 48 | Wife |
| C_1890 | F | Healthy | 67 | 73 | 164 | 48 | Wife |

Statistical Analysis

Data are expressed as mean±SEM. p values<0.05 were considered significant (*p<0.05; **p<0.05; ***p<0.005; ****p<0.0005). Pairwise comparisons were performed using Student's t test. Mann-Whitney U test was used when the distribution was not known to be normal. Comparison between multiple groups was performed using ANOVA, and FDR correction was used to adjust for multiple comparisons. We analyzed the effect of Abx over time in control and SOD1-Tg mice by modeling neuro-phenotypical measurements (rotarod, grip test score and neurological score) as a function of time and treatment in a time-depended manner using a linear regression:

Phenotype˜time+time×treatment+time×genotype+time×treatment×genotype

where time is the day (60, 80, 100, 120 and 140), treatment (±Abx) and genotype (WT or SOD1-Tg) are binary indicators. Significance of treatment is then inferred by the p-value of the time×treatment predictor. For this analysis we used python statsmodels.api.ols version 0.8.0 statsmodels.

Microbial abundance change over time was evaluated using linear regression:

OUT˜time+time×genotype

The significance of genotype effecting OUT abundance was inferred by the p-value of the time x genotype predictor after 5% FDR correction for multiple OTUs.

To analyze KOs of the nicotinamide and tryptophan metabolic pathways KO levels between groups were compared using Mann Whitney U ranksum test. For this analysis the python stats.HigherLevelRanksum.directed_mannwhitneyu was used.

Results

An Altered Gut Microbiome Exacerbates Motor Symptoms in an ALS Mouse Model

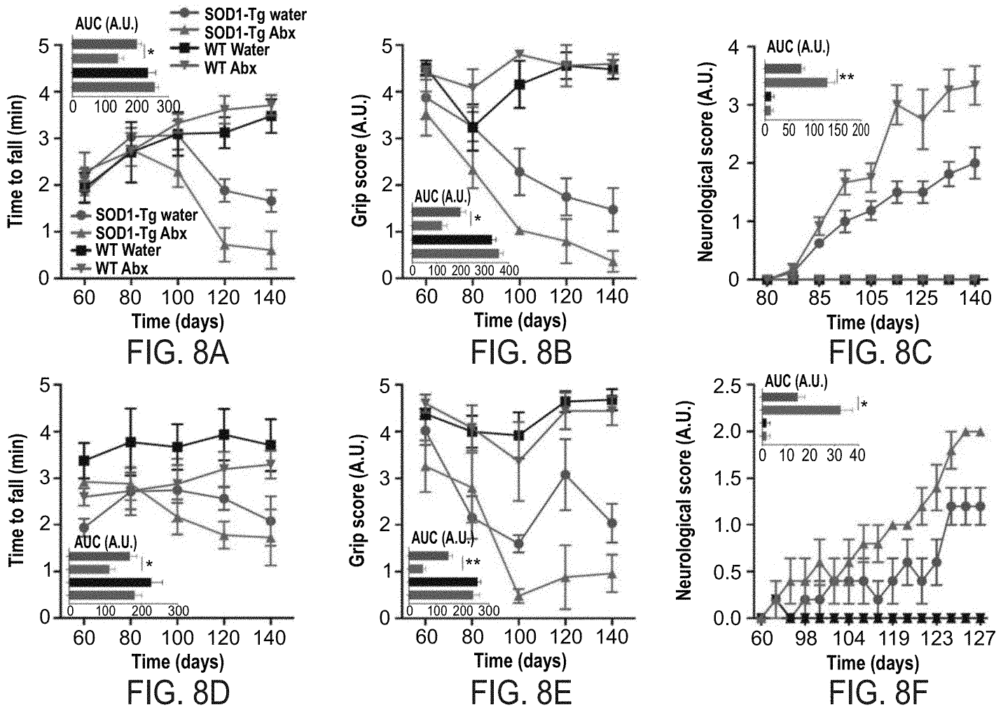

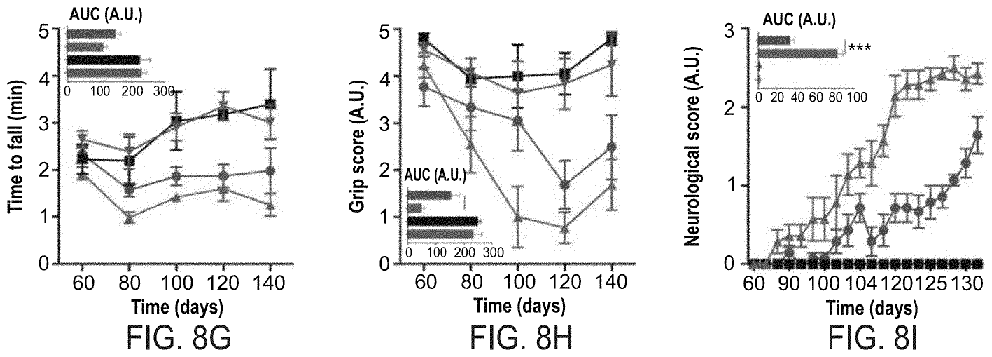

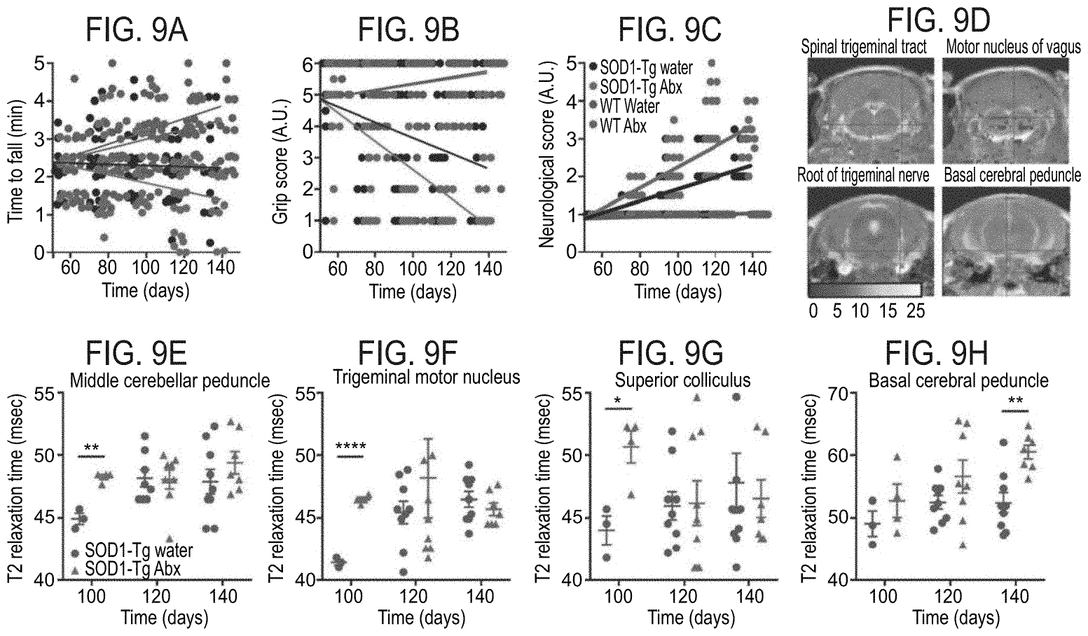

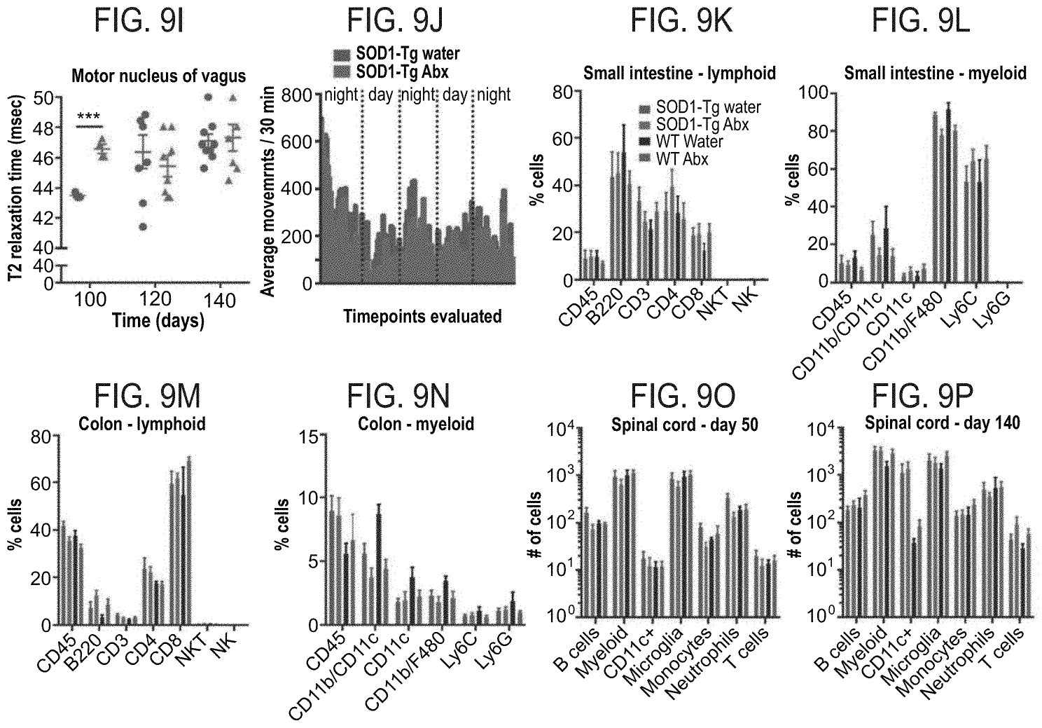

To assess the potential modulatory role of the gut microbiome in ALS we used the high copy number mSOD1 G93A (herein “SOD1-Tg”) mouse model for amyotrophic lateral sclerosis (ALS). We began our investigation by depleting the gut microbiome of male and female SOD1-Tg or littermate controls at our facility, by administrating a combination of vancomycin (0.5 g/l), ampicillin (1 g/l), neomycin (1 g/l), and metronidazole (1 g/l) (broad-spectrum antibiotics, Abx), that have been consistently shown to markedly deplete the indigenous microbiome in mice²⁵ starting at the age of 40 days (FIG. 1A). Motor abilities were quantified using multiple methods, namely rotarod locomotor test²⁶, hanging-wire grip test²⁷ and neurological scoring²⁸. Throughout the project, key repeat experiments were independently scored by two blinded researchers. Surprisingly, Abx treatment was associated with a significant and substantial exacerbation of motor abnormalities throughout ALS progression, compared to the water-treated SOD1-Tg group. Both the pooled results (N=15-30 mice per group, FIGS. 1B-D) and independent results of each of the repeats ((N=5-10 mice in each group of each repeat, three independent repeats, FIGS. 8A-I) demonstrated worsened results in the rotarod locomotor test (FIG. 1B, FIG. 8A, 8D and 8G), the hanging-wire grip test (FIG. 1C, FIG. 8B, 8E and 8H) and neurological scoring (FIG. 1D, FIG. 8C, 8F and 8I). Notably, Abx treatment did not affect rotarod or grip test performances in WT littermate controls at our vivarium, as compared to non-Abx-treated WT mice (FIGS. 1B-D and FIGS. 8A-I). A linear regression analysis further supported the statistically-significant negative effect of Abx treatment on these neuropathological measurements in SOD1-Tg mice (FIGS. 9A-C).

In agreement with these findings, spinal cord histopathological analysis of neuronal numbers (using luxol fast-blue staining) at day 140 revealed a significant reduction in motor neuron cell counts in Abx-treated compared to water-treated SOD1-Tg mice (FIGS. 1E-F), suggesting an increased motor neuron cell-death following chronic Abx exposure. Moreover, T₂-weighted magnetic resonance imaging (MRI) of the murine brain stem in areas known to degenerate in the SOD1-Tg model (FIG. 9D^{29,30}) demonstrated a prolonged T₂ relaxation time Abx-treated SOD1-Tg mice (FIG. 1G-I, FIG. 9D-I), indicative of higher levels of free water, enhanced brain atrophy and neurodegeneration³¹. Automated home-cage locomotion system revealed a significant reduction (p=0.03) in the activity of Abx-treated SOD1-Tg mice on day 100 compared to water-treated SOD1-Tg controls (FIG. 9J). Abx-induced aggravation in motor function of SOD1-Tg mice was not associated with alterations of the main immune cell sub-populations in spinal cord (including activated microglia), small intestine or colon lamina propria, compared to water-treated SOD1-Tg mice (FIG. 9K-P), suggesting that the Abx-associated phenotypic differences were not mediated by marked immune aberrations.

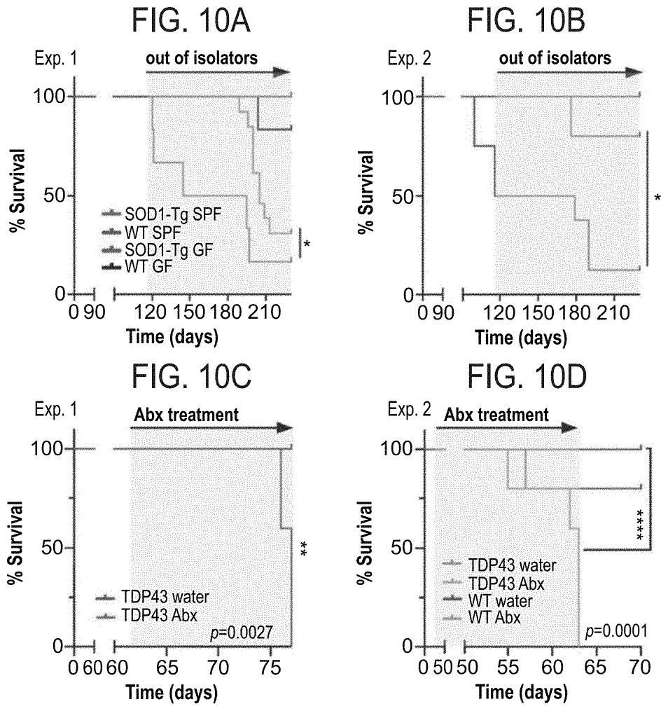

Importantly, rederivation attempts of SOD1-Tg mice into the germ-free setting was associated with high-rates of mortality of SOD1-Tg but not of WT littermate controls (failed rederivation attempts of 30 pregnant dams over a period of 18 months). Once rederivation succeeded, GF SOD1-Tg mice featured significantly enhanced mortality as compared to GF WT littermates or to colonized SOD1-Tg mice (FIG. 1J, pooled results, N=9-22 mice per group, FIGS. 10A-B, two independent repeats, N=5-13 per group). Enhanced mortality remained present even when GF mice were spontaneously colonized at Day 115, suggesting that microbial drivers impact ALS progression at an earlier disease stage. Moreover, microbiome depletion by Abx treatment substantially and significantly enhanced mortality in an additional ALS mouse model, TDP43-Tg mice (FIG. 1K for pooled results, and FIGS. 10C-D for the individual repeats), suggesting that this detrimental microbiome depletion effect was not confined to SOD1 mutations. Collectively, these results indicated a potential detrimental effect of Abx-mediated microbiome alteration (or its absence in GF mice) at our vivarium, on ALS manifestation in SOD1-Tg mice, suggesting that a locally dysbiotic gut microbiome configuration may modulate disease progression in this model.

SOD1-Tg Mice Develop a Vivarium Dependent Pre-Clinical Dysbiosis

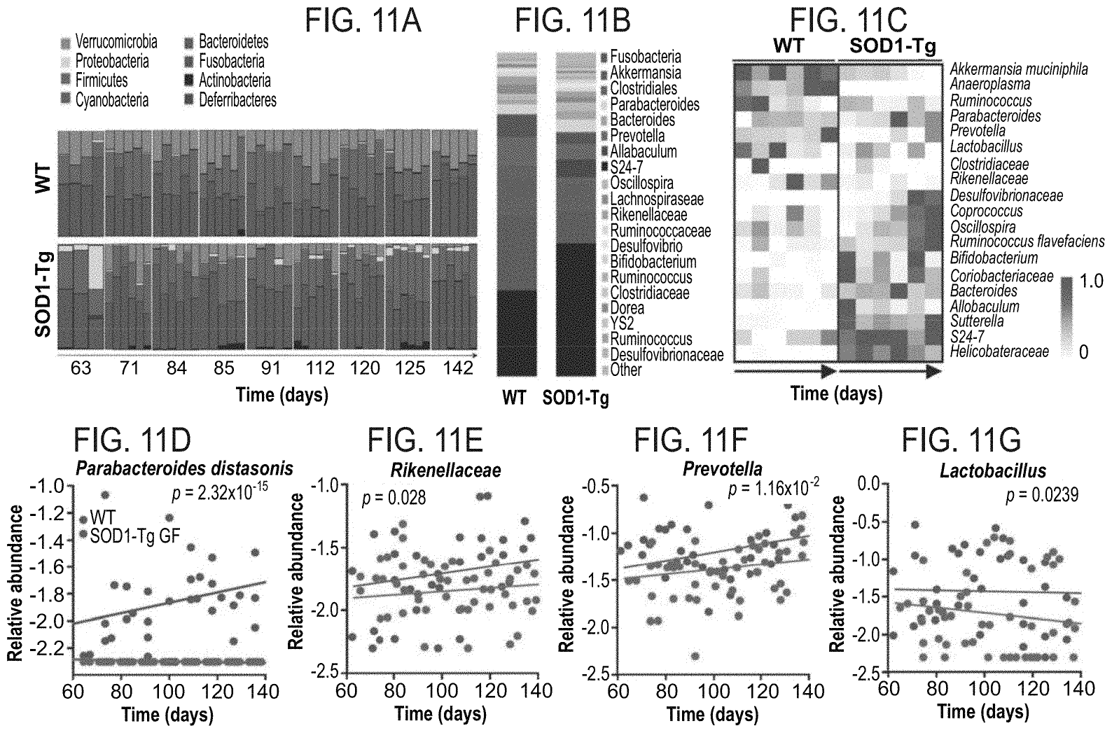

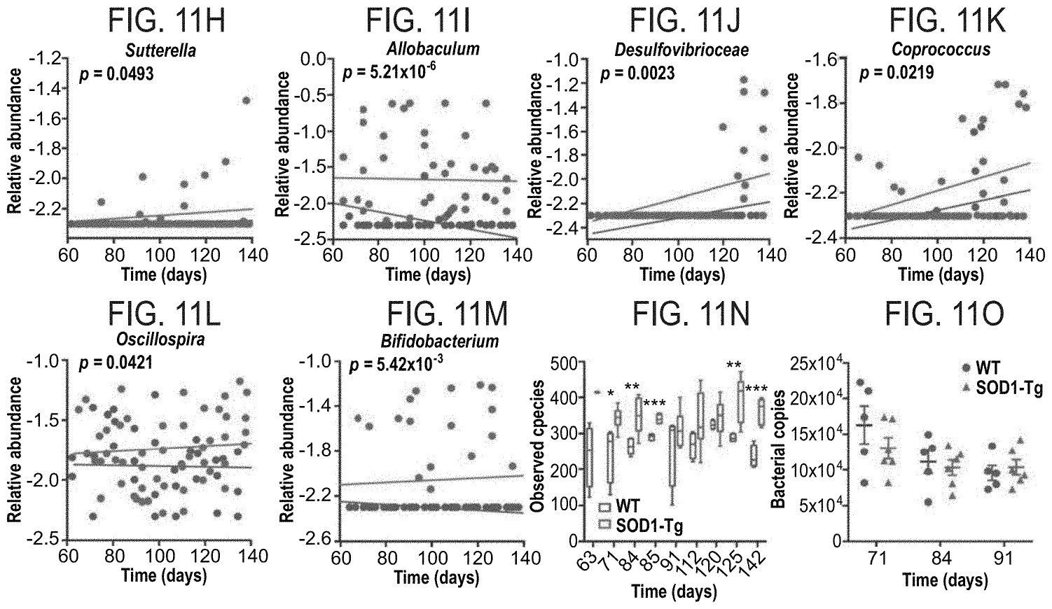

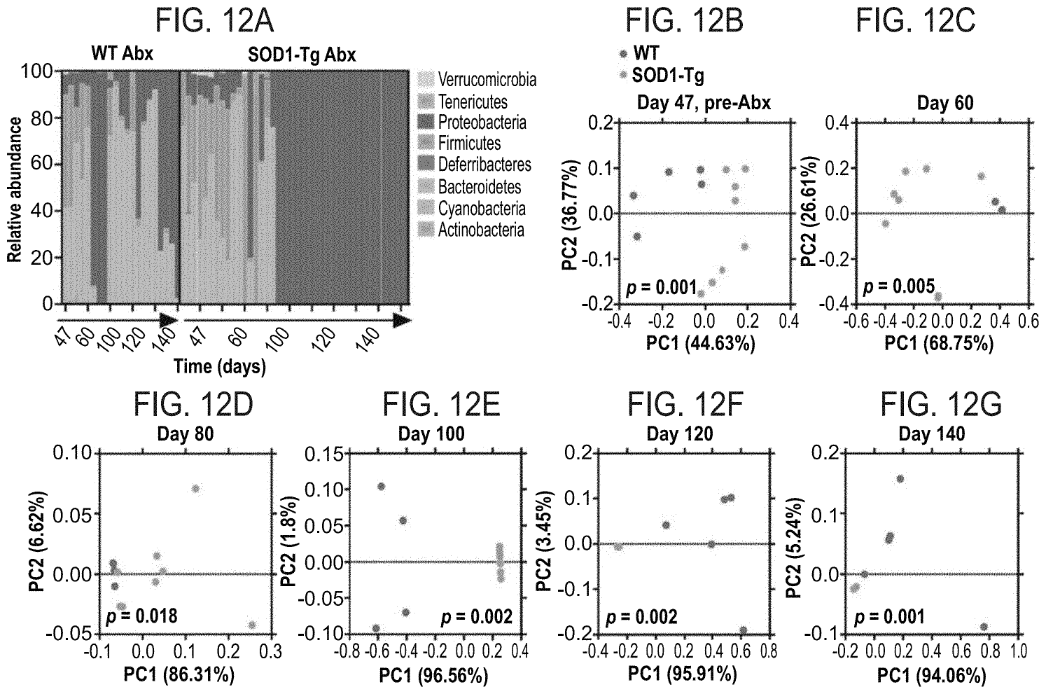

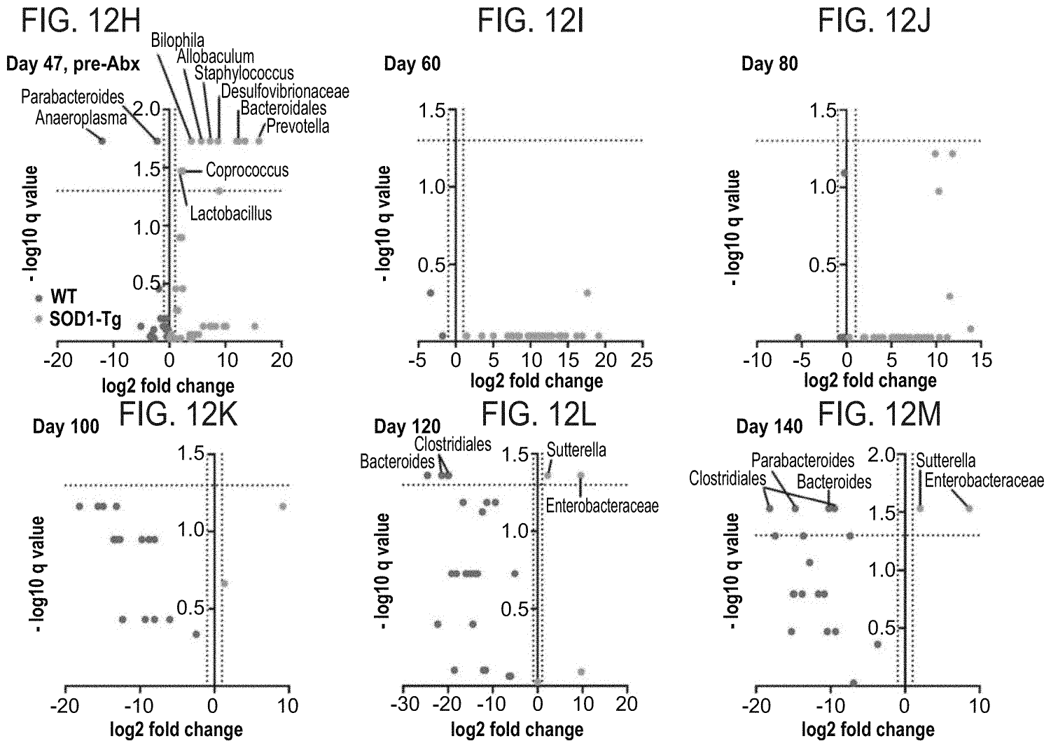

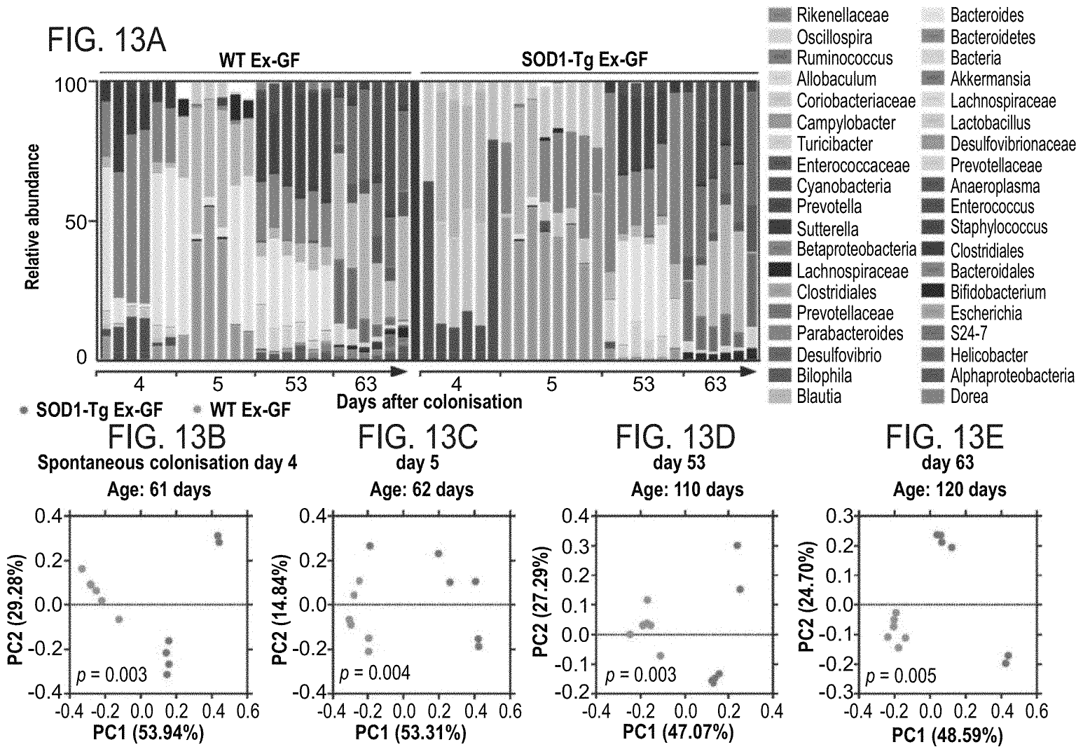

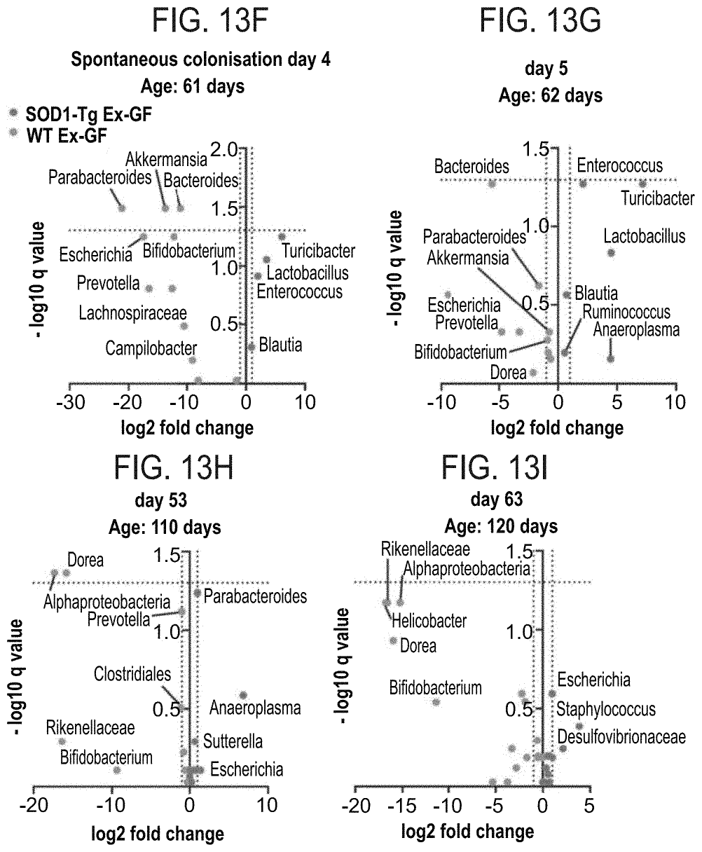

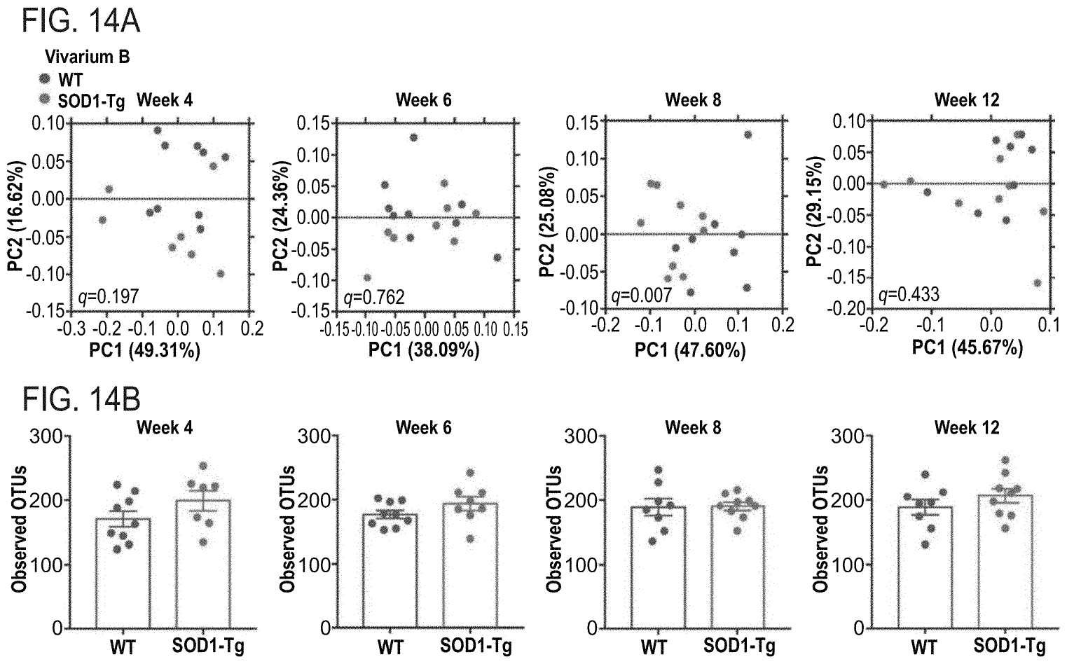

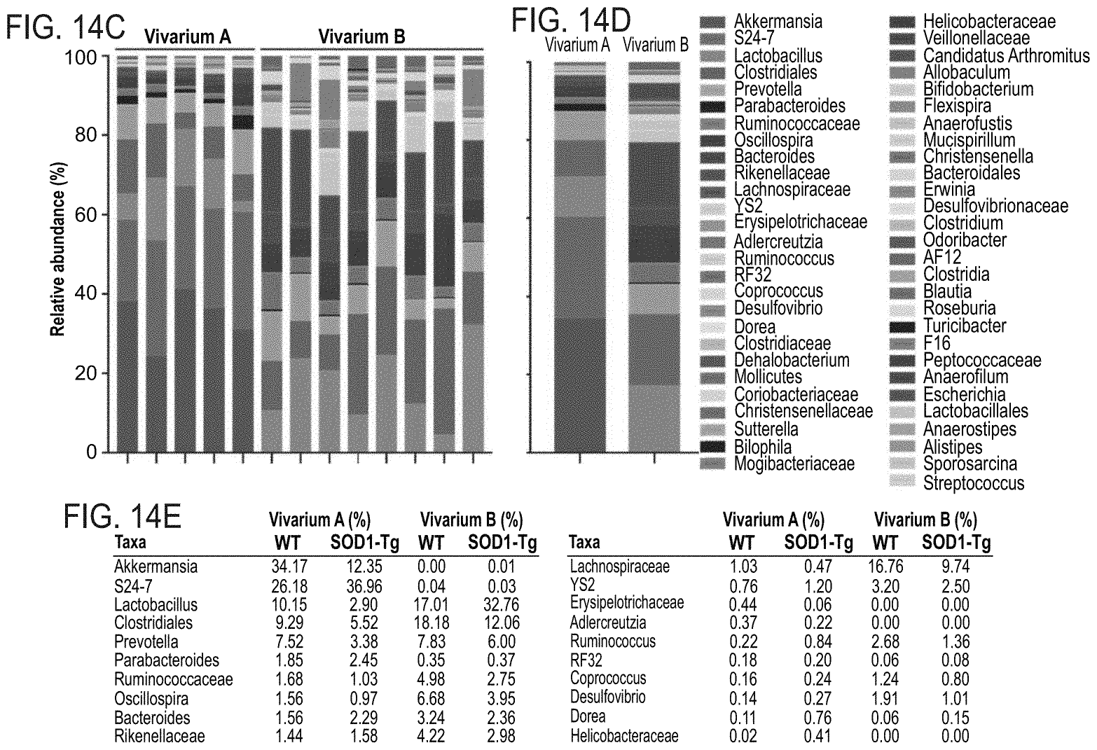

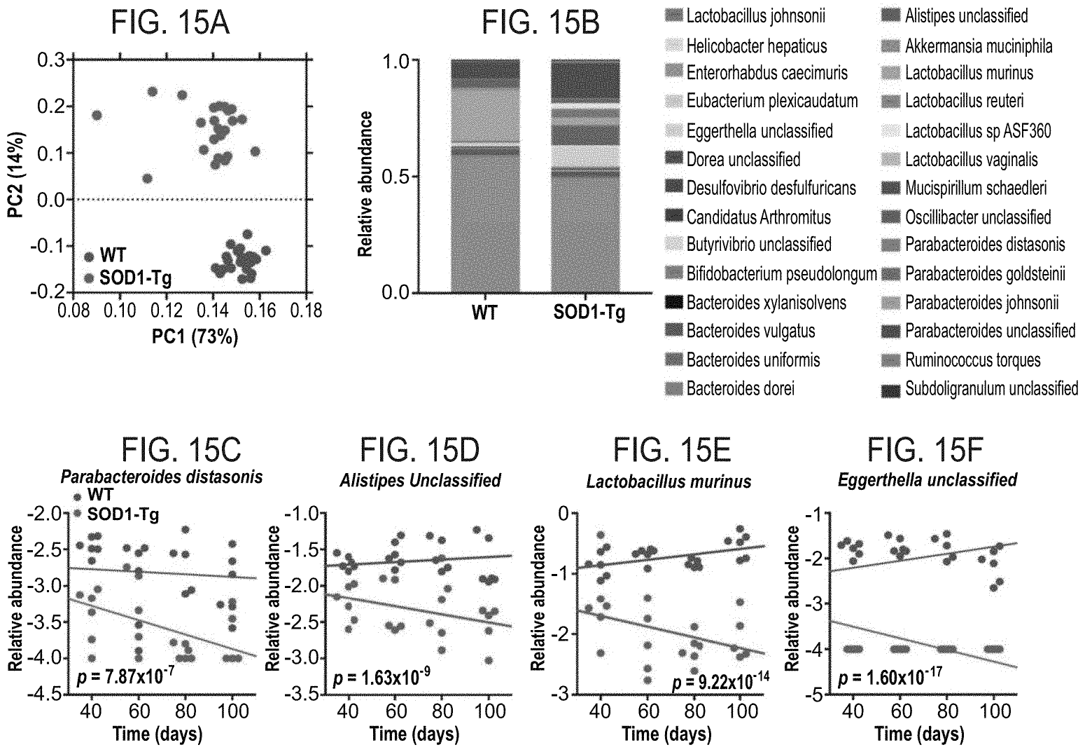

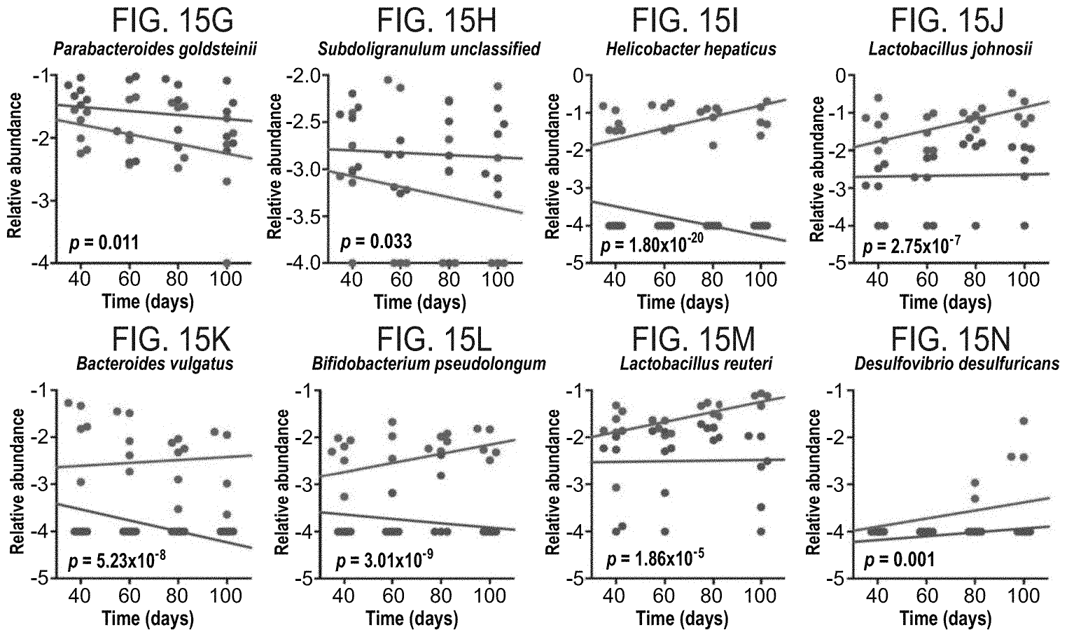

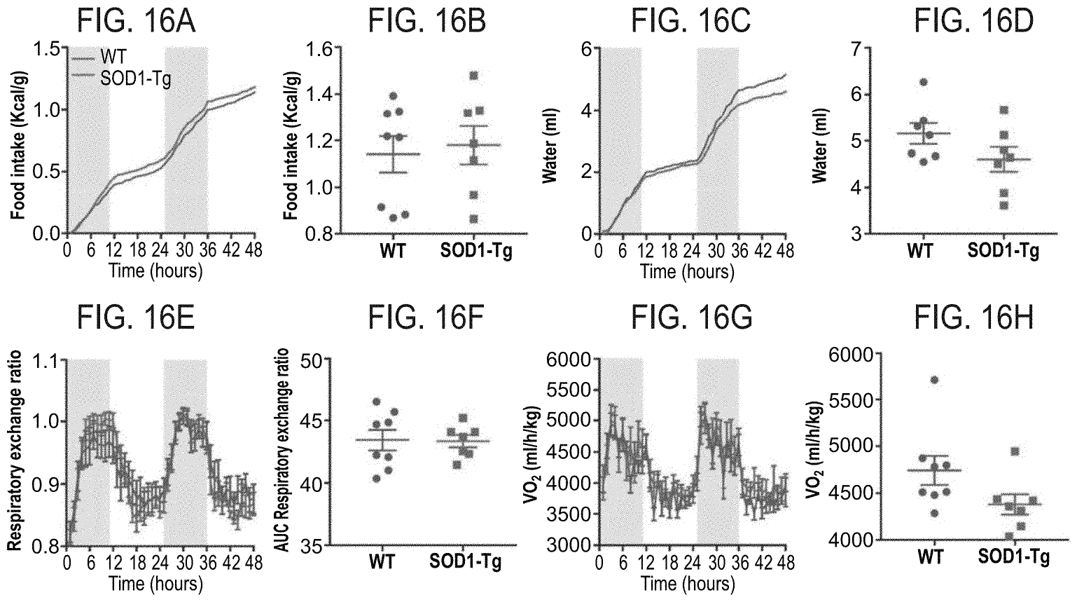

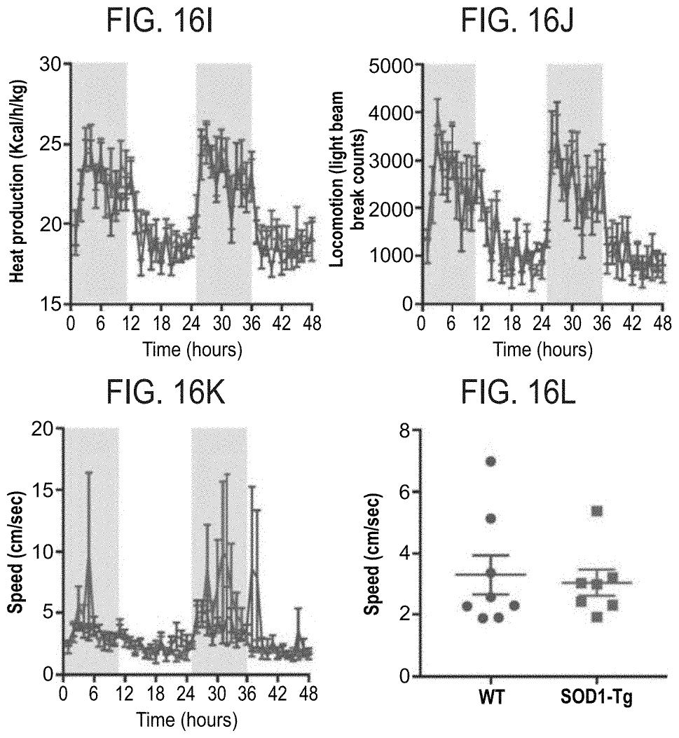

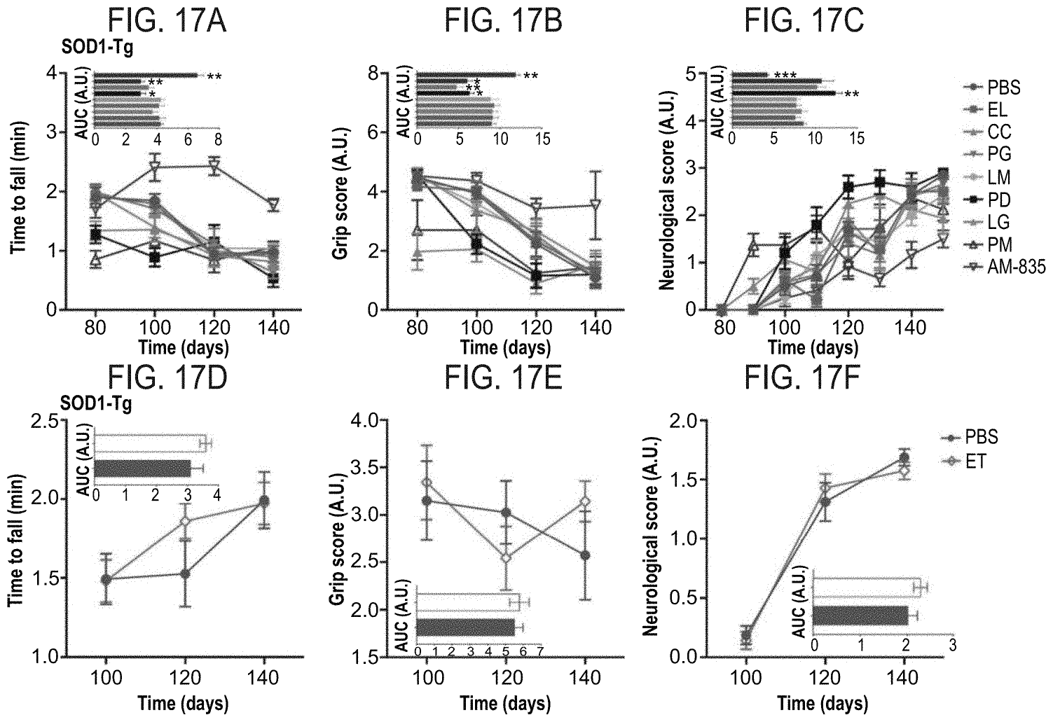

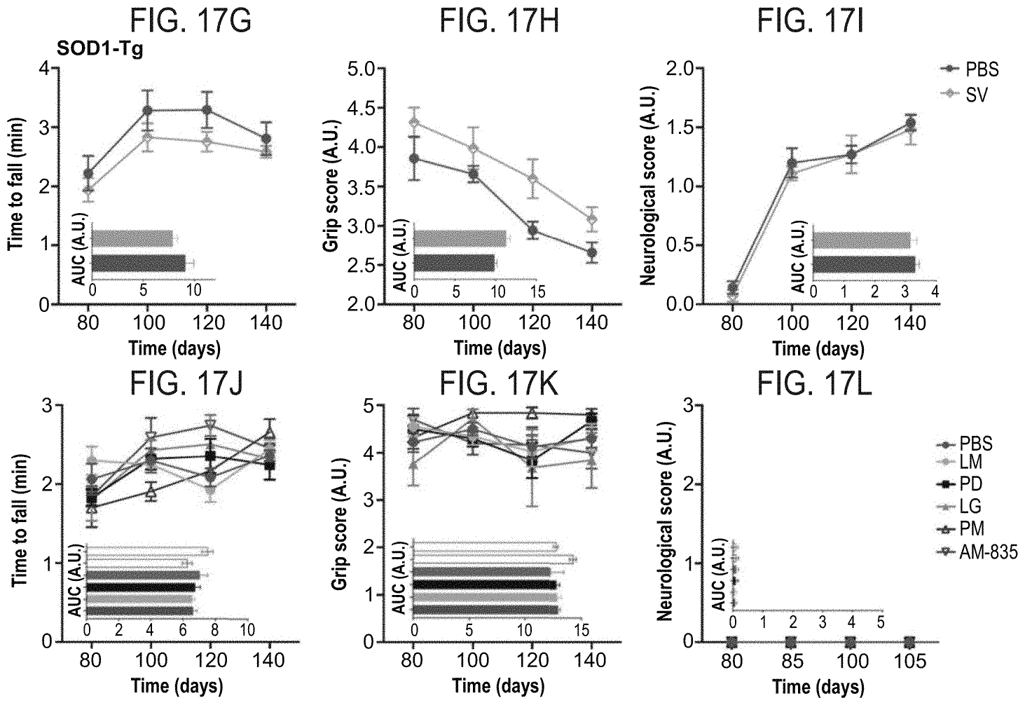

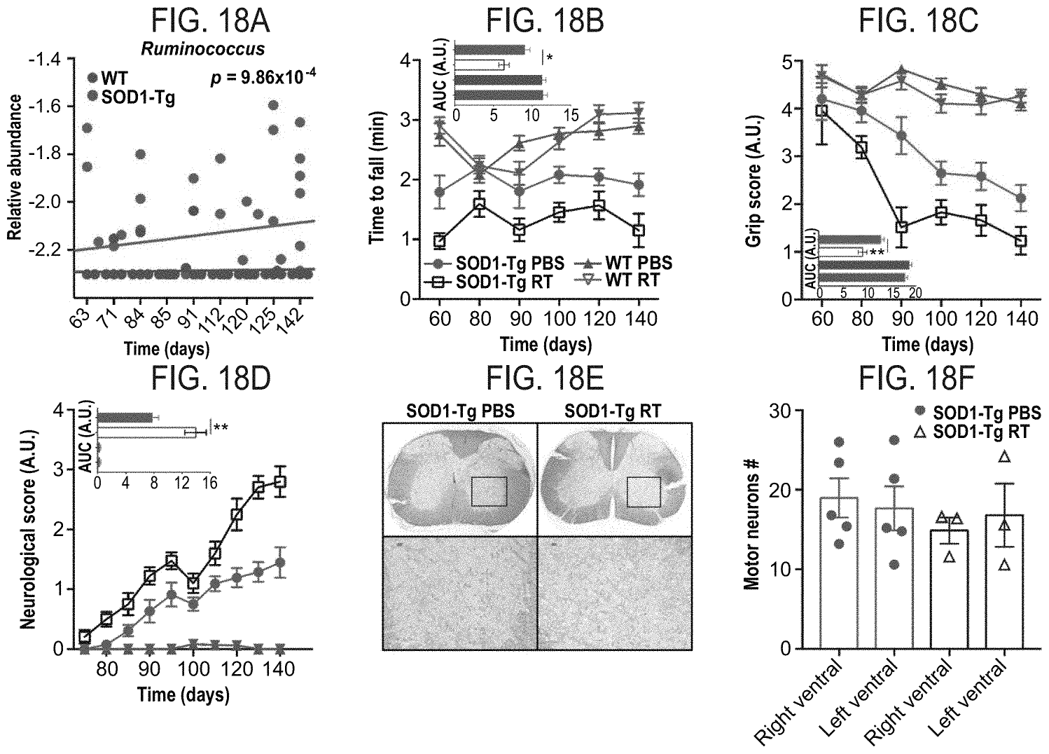

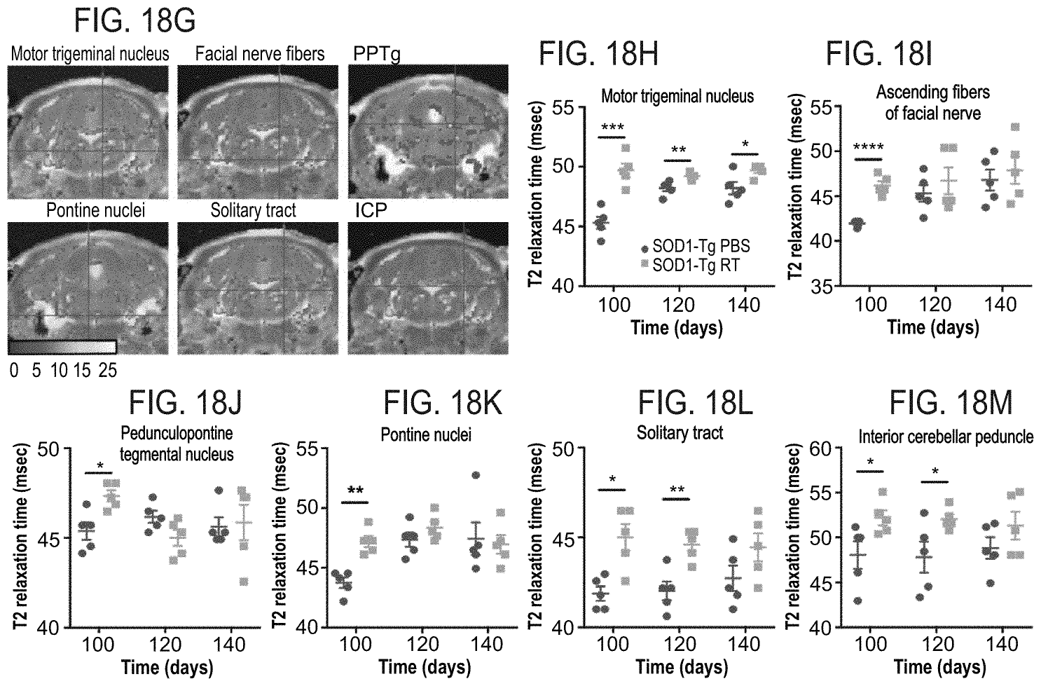

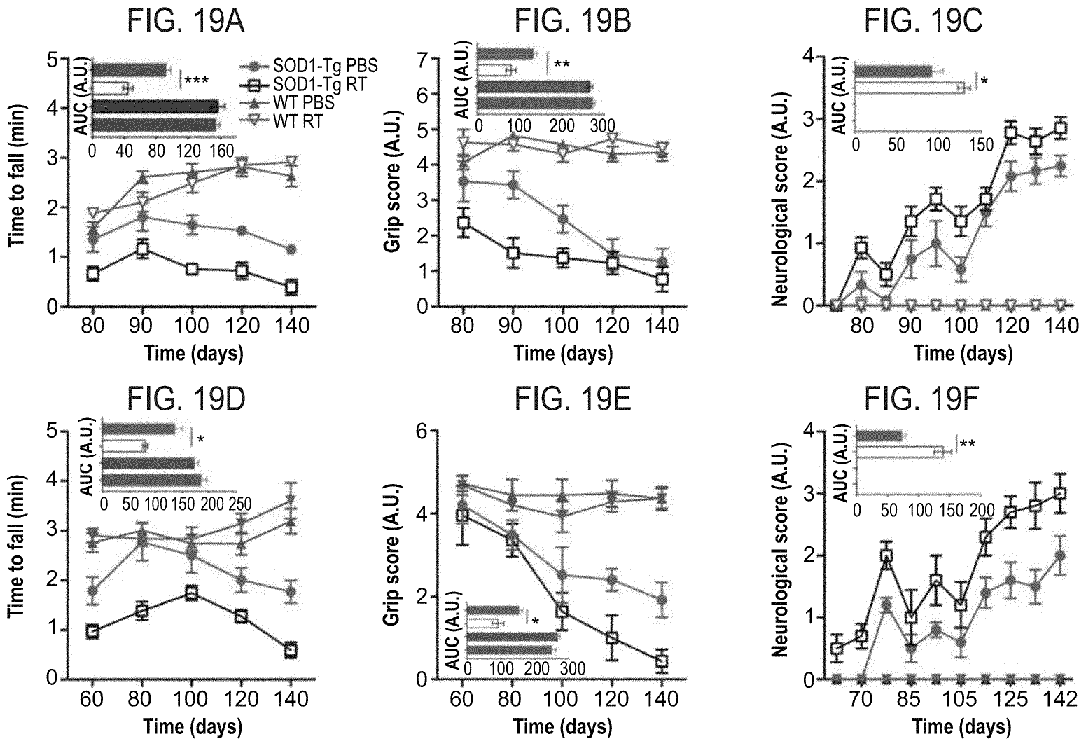

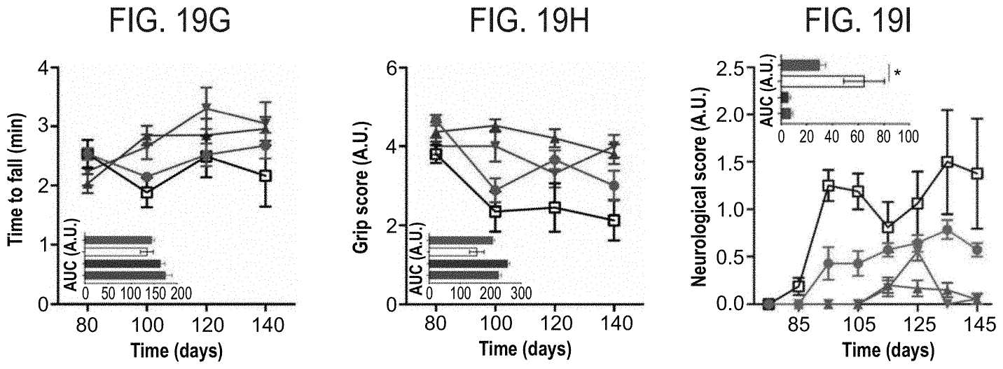

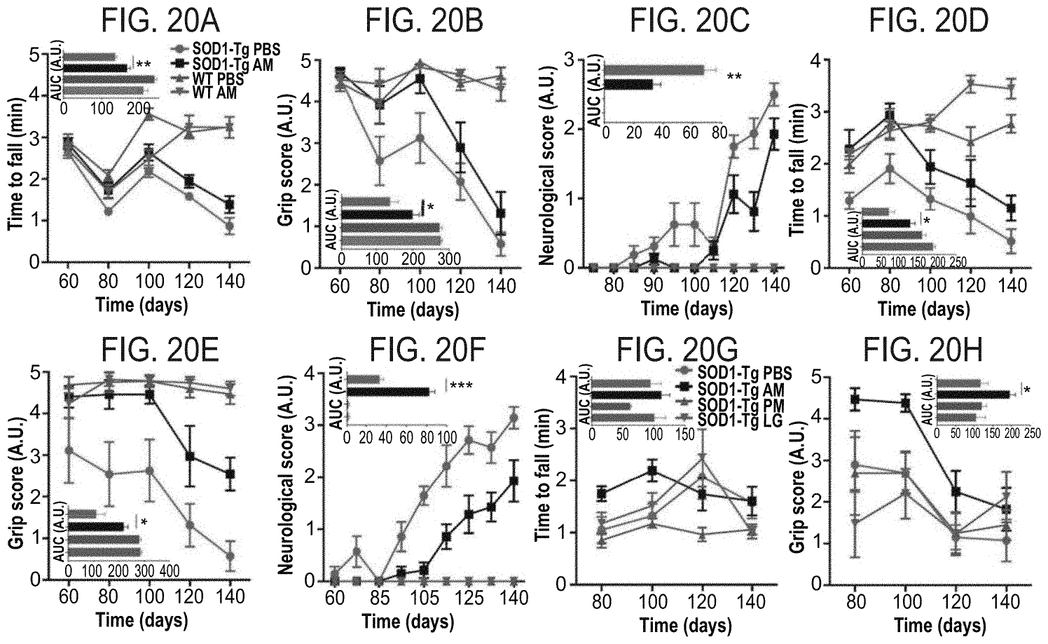

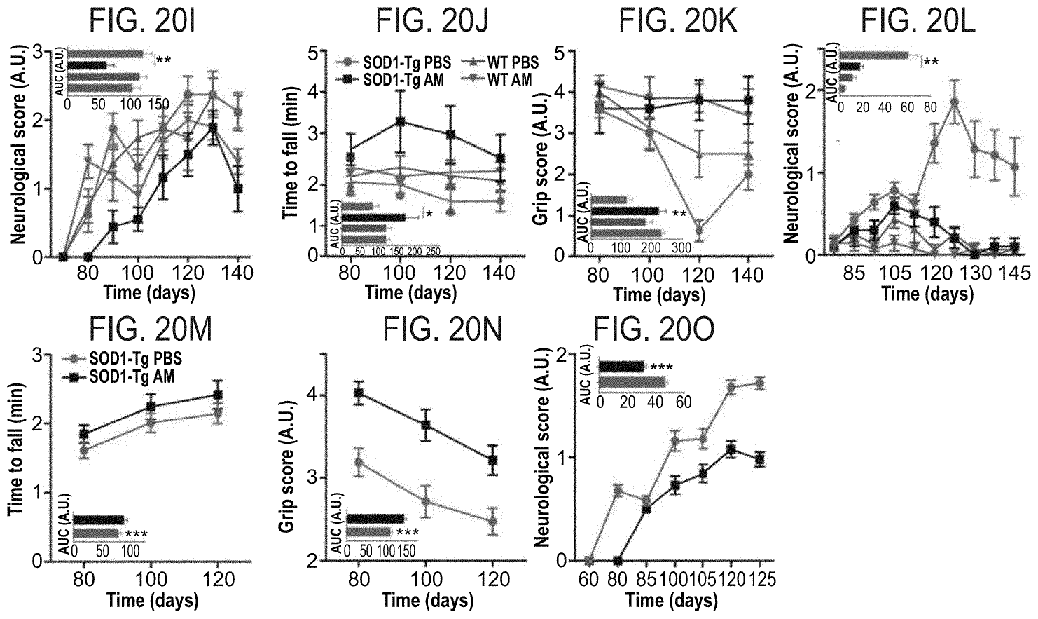

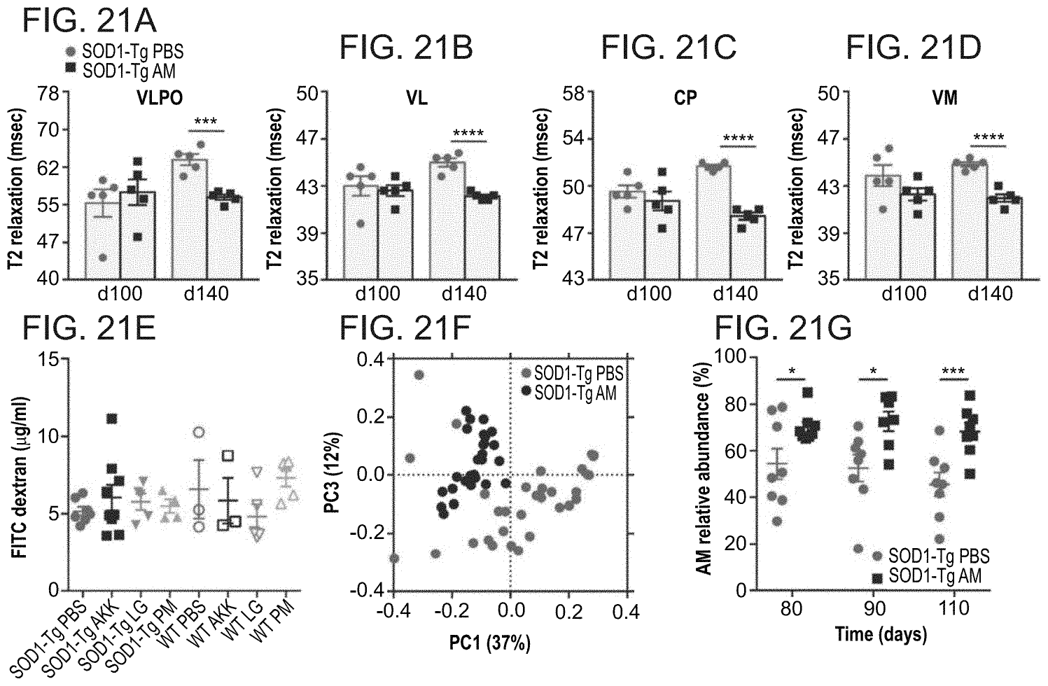

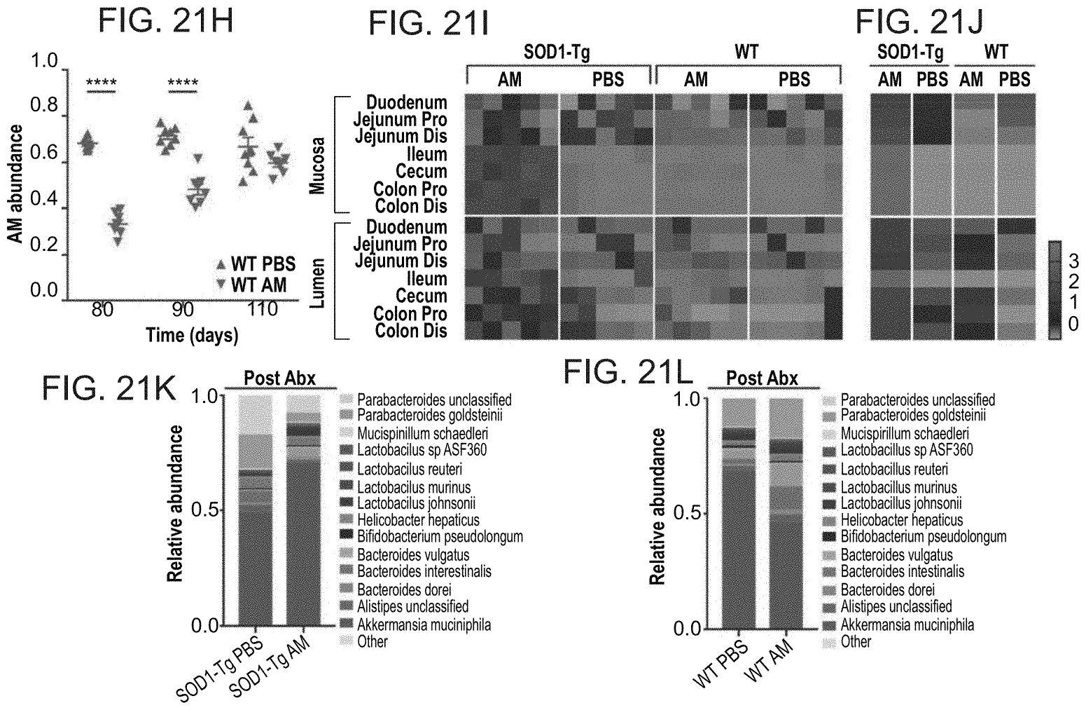

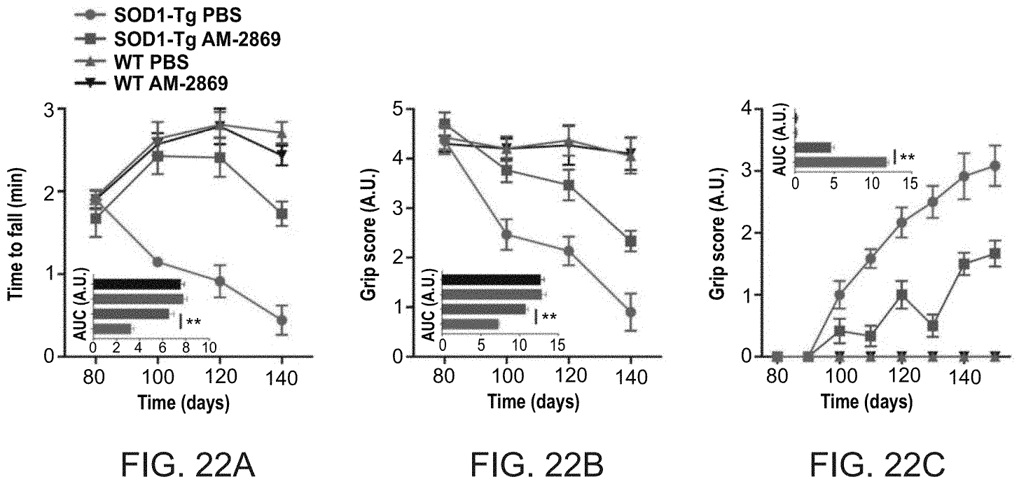

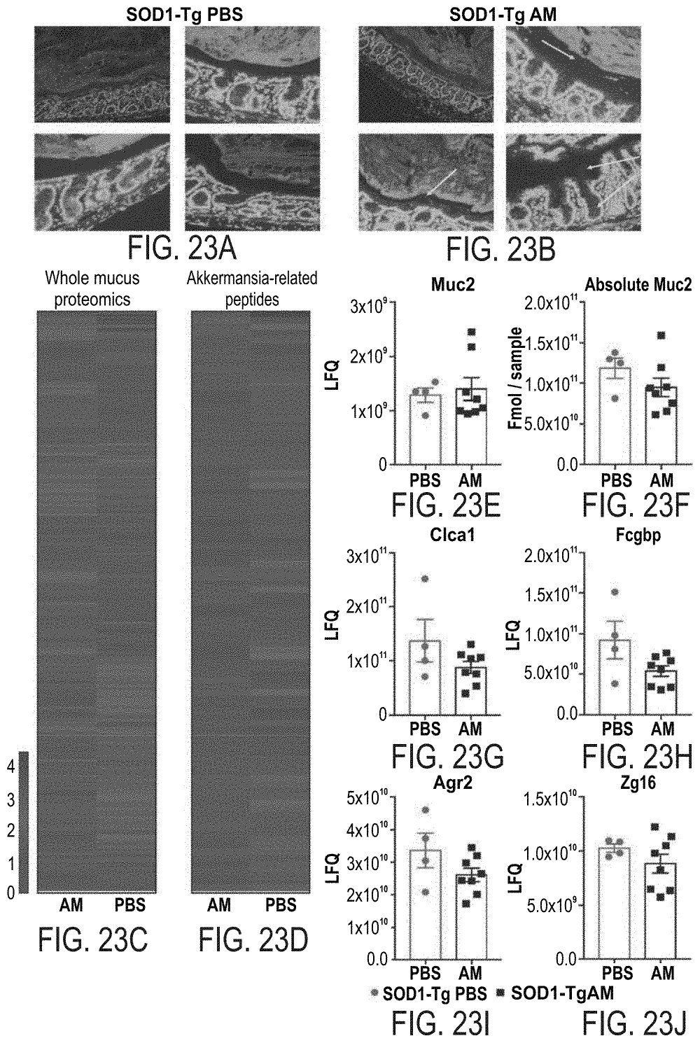

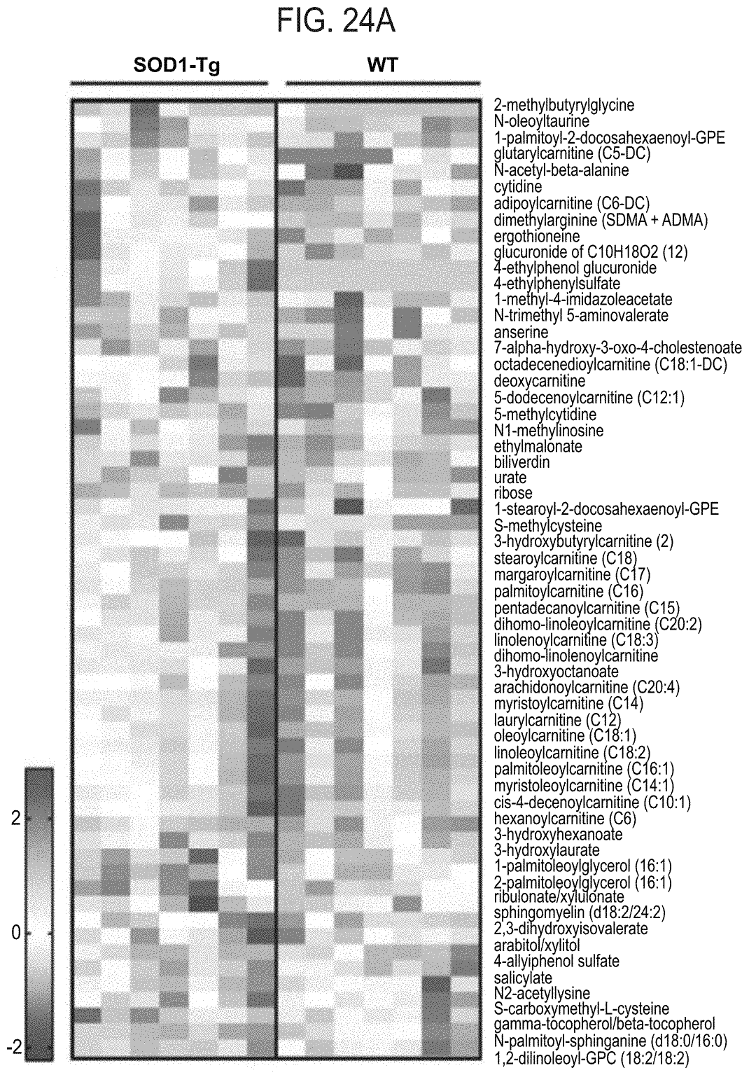

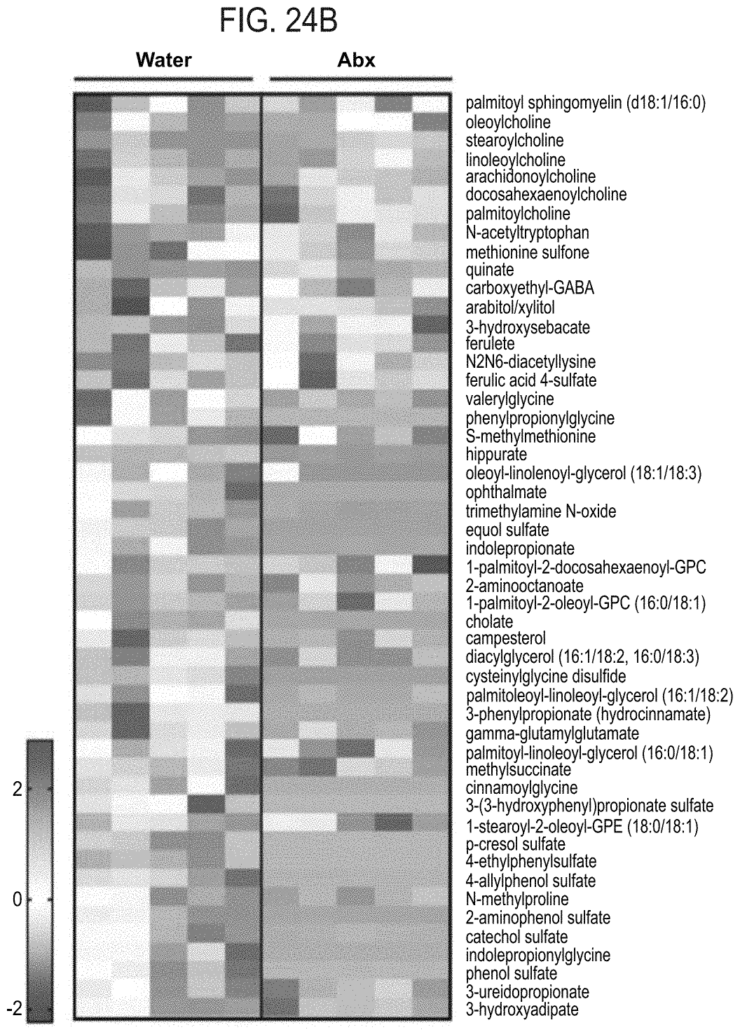

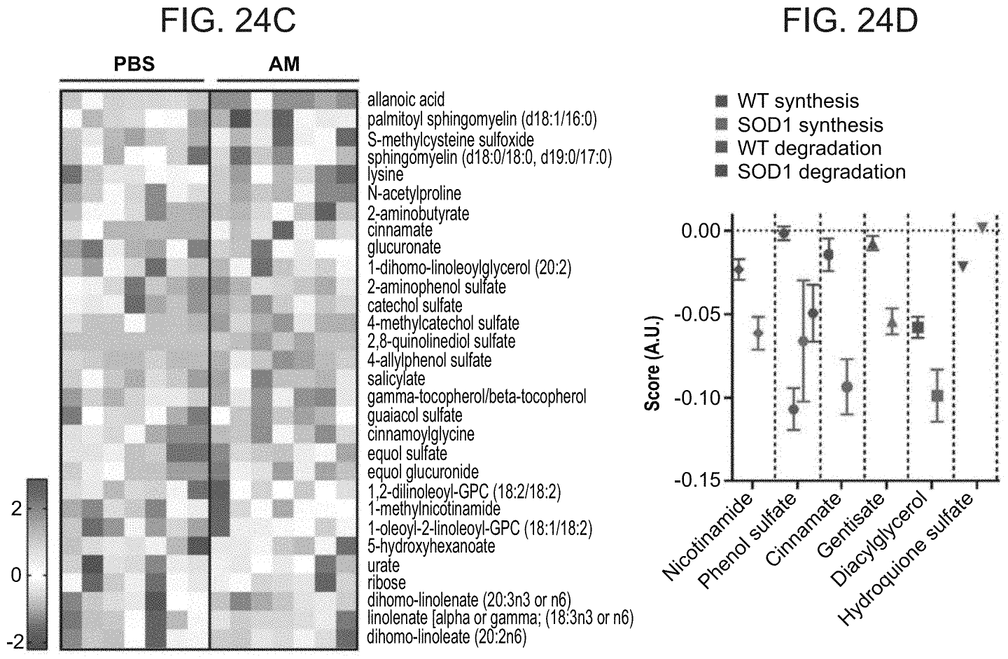

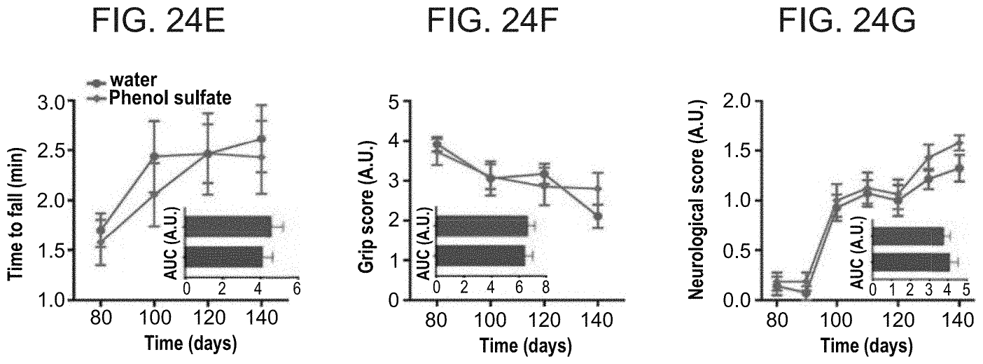

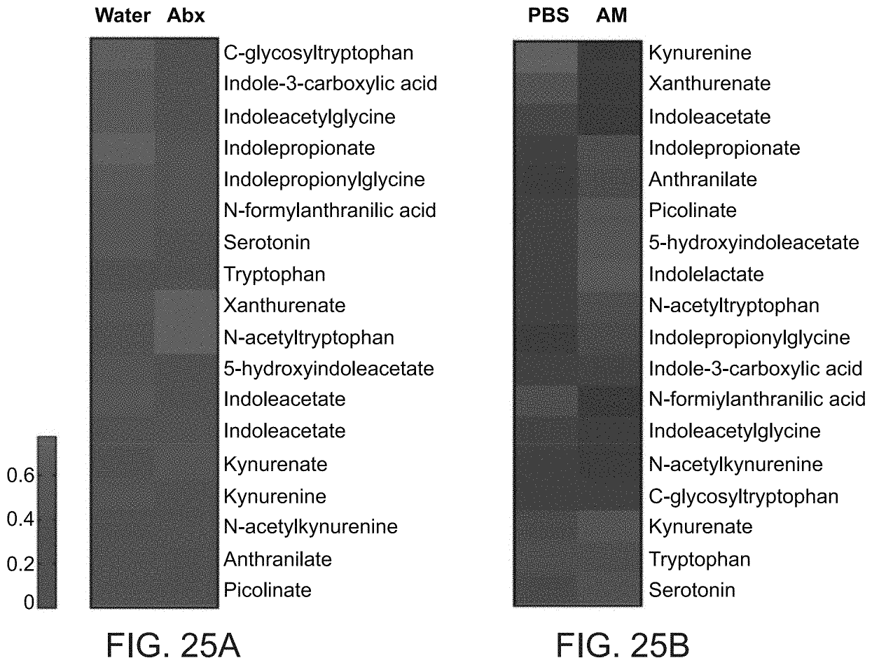

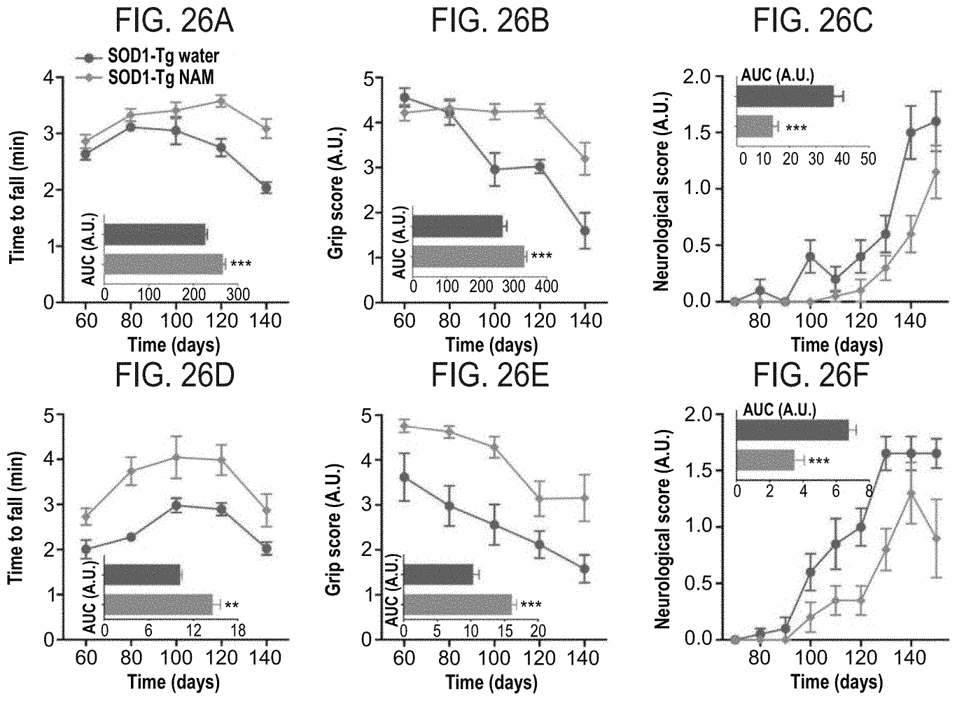

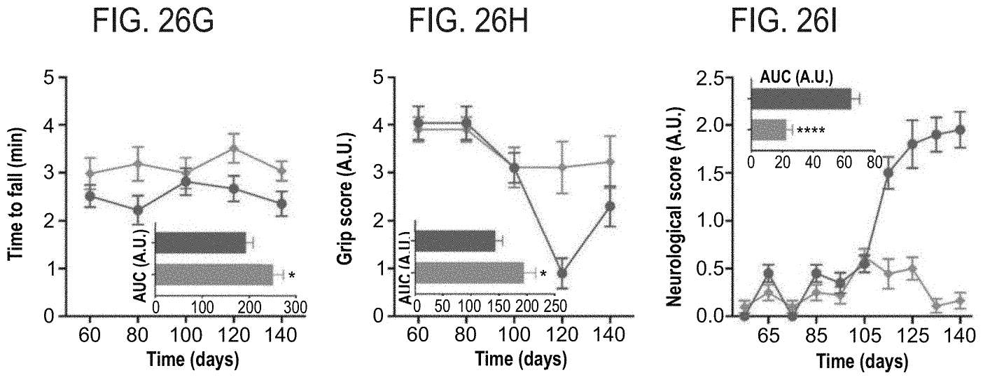

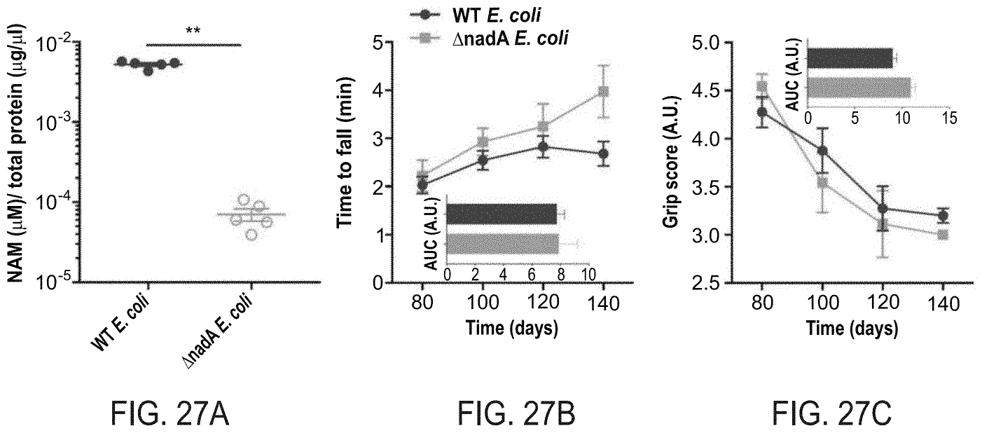

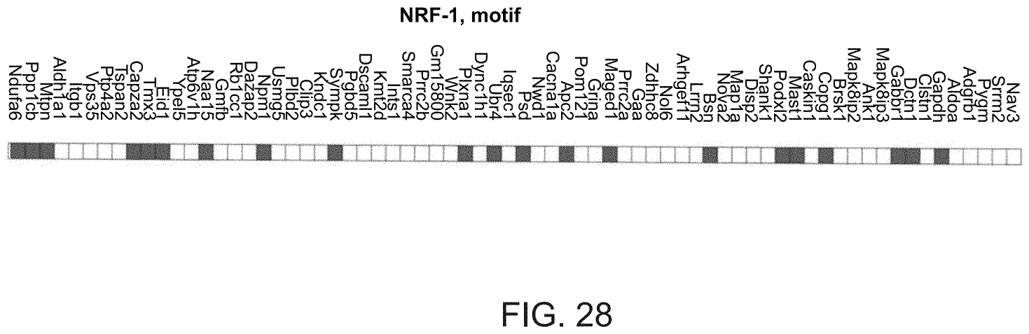

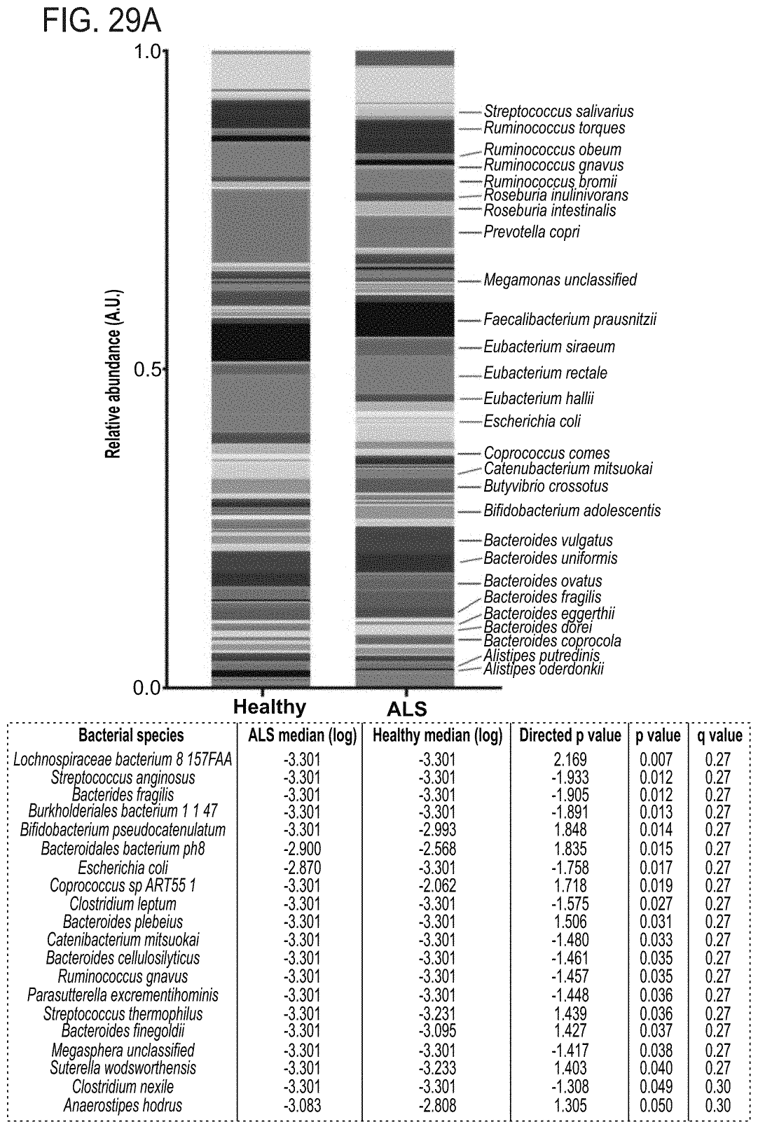

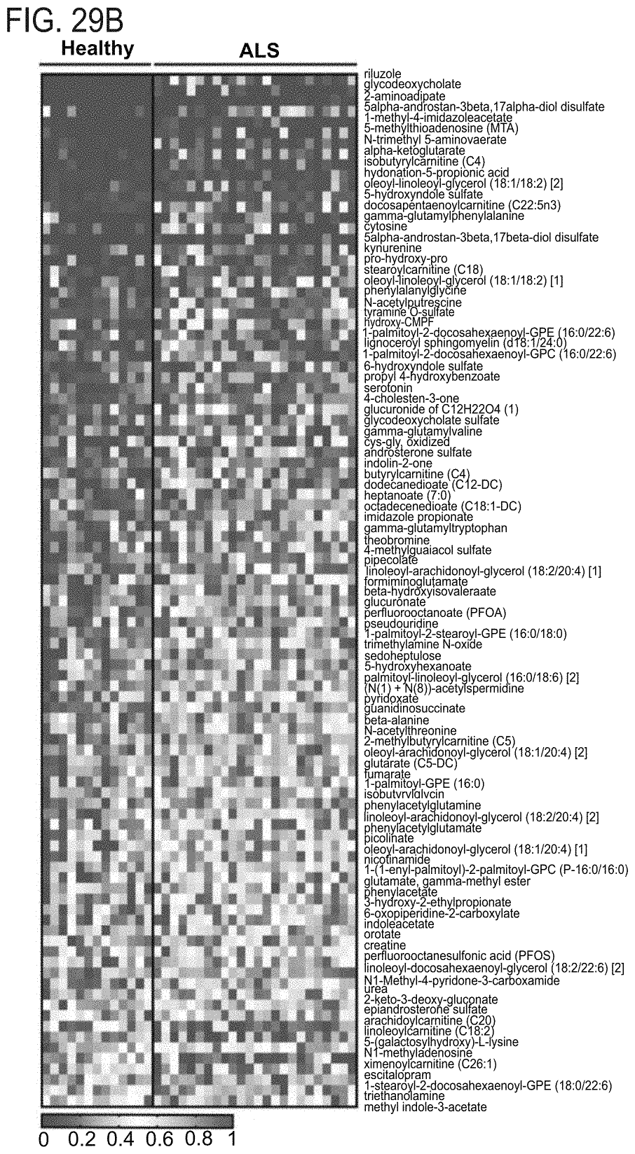

These suggested microbial-mediated effects on ALS neuropathology in the SOD1-Tg model at our vivarium presented an opportunity to identify locally-prevalent commensal strains potentially modulating ALS course. Indeed, assessment of fecal microbiome composition and function by 16s rDNA sequencing in SOD1-Tg and WT littermate controls at our facility indicated an early and significant microbiome compositional difference that persisted during disease course (FIGS. 2A-C, FIGS. 11A-C). Notably, at our vivarium, dysbiosis in SOD1-Tg mice was mainly driven by the genera Akkermansia, Anaeroplasma, Prevotella, Parabacteroides, Rikenellaceae and Lactobacillus, which were all significantly reduced in SOD1-Tg feces as compared to WT littermate controls FIGS. 11C-G), while Ruminoccocus, Desulfovibrioaceae, Allobaculum, Sutterella, Helicobacteraceae, Coprococcus and Oscillospira were enriched in their 16S rDNA abundances in the SOD1-Tg fecal microbiome (FIGS. 11H-M). Moreover, the total number of observed genera (alpha diversity) was higher in the SOD1-Tg stool at all time-points (FIG. 11N), indicating an altered community structure in SOD1-Tg mice compared to WT littermates. However, total fecal bacterial load did not vary between SOD1-Tg and WT controls (FIG. 110). Moreover, even the gut microbiome configuration of Abx-treated SOD1-Tg and their WT littermate controls at our vivarium yielded significantly differential microbiome compositions in all the examined time-points across disease progression (FIGS. 12A-G), driven by blooming of Bacteroides, Parabacteroides and Clostridiales genera in the Abx-treated WT microbiomes, and of Sutterella and Enterobacteraceae in the Abx-treated SOD1-Tg mice (FIGS. 12H-M). Importantly, spontaneous colonization of GF SOD1-Tg and WT littermates at our vivarium was associated with the development of de-novo dysbiosis (FIGS. 13A-I), while these facility-dependent dysbiotic differences were not observed in a second non-barrier (non-SPF) vivarium featuring a near-absence of Akkermansia, Parabacteroides, Erysipelotrichaceae and Helicobacteraceae (FIGS. 14A-E). Overall, these facility-dependent changes suggested that a combination of murine-ALS genetic susceptibility, coupled with a locally-prevalent commensal signature drive early pre-clinical dysbiosis possibly contributing to ALS modulation at this facility.

To further assess species-level compositional and functional microbiome differences associated with ALS progression at our vivarium, we conducted a shotgun metagenomic sequencing of the fecal microbial DNA of SOD1-Tg mice, as compared to WT littermates at different time points. Indeed, using MetaPhlan2, significant differences were noted in the microbiome composition of SOD1 mice as compared with littermate controls (FIG. 2D and FIG. 15A-B), stemming from multiple species-level taxonomical differences. For example, Parabacteroides distasonis, Alistipes unclassified, Lactobacillus murinus, Eggerthella unclassified, Parabacteroides goldsteinii, Subdoligranulum unclassified and Akkermansia muciniphila (FIGS. 15C-H and FIG. 3A) were significantly decreased in the SOD1-Tg microbiome, whereas Helicobacter hepaticus, Lactobacillus johnsonii, Bacteroides vulgatus, Bifidobacterium pseudolongum, Lactobacillus reuteri and Desulfovibrio desulfuricans (FIGS. 15I-N) were enriched compared to WT littermate controls. Functionally, SOD1-Tg and WT fecal bacterial metagenomes clustered separately with respect to microbial genes (for PC1: day 40, p=0.0002, day 60, p=0.0002, day 80, p=0.0005, day 100, p=0.0005, KEGG orthology, KO, FIG. 2E), including a marked reduction in representation of genes encoding enzymes participating in tryptophan metabolism (FIGS. 2F-G) and substantial alterations in genes encoding enzymes involved in nicotinamide and nicotinate metabolism (FIG. 2H). To rule out that these early microbiome effects were secondary to altered metabolism in SOD1-Tg mice, we performed a detailed metabolic assessment at the pre-clinical day 60, and found no significant changes in food and water intake, respiratory exchange ratio, oxygen consumption, locomotion, and heat production (FIG. 16A-L).

Collectively, these results demonstrated that single-genotype-housed SOD1 mice diverge in their gut microbial composition and function from their WT littermate configuration at our vivarium, even before the appearance of clinical motor neuron dysfunction symptoms.

Commensal Microbe Contribution to ALS Exacerbation

We next sought to determine possible causal relationships between the above vivarium-dependent differentially-abundant gut commensal microbes and modulation of murine ALS-associated motor function. In all, we tested 11 strains, including Eggerthella lenta, Coprobacillus cateniformis, Parabacteroides goldsteinii, Lactobacillus murinus, Parabacteroides distasonis, Lactobacillus gasseri, Prevotella melaninogenica, Eisenbergiella tayi (member of the Lachnospiraceae family), Subdoligranulum variabile, Ruminococcus torques and Akkermansia muciniphila, all suggested by our composite 16S rDNA and shotgun metagenomic analysis to be correlated with severity of ALS progression in the SOD1-Tg model at our vivarium (FIGS. 11A-O and FIGS. 15A-N). To this aim, we mono-inoculated anaerobic cultures of each of the above strains (stationary phase O.D.=0.4-0.7) into Abx pre-treated SOD1-Tg and WT mice, by repeated oral administration at 6 day-intervals for a total of 15 treatments. Mono-colonization of these mice with most of the indicated bacteria did not affect ALS symptoms (FIGS. 17A-L). Supplementation of Abx-treated SOD1-Tg mice with two strains, Parabacteroides distasonis (PD, FIGS. 17A-L) and Ruminococcus torques (RT, FIGS. 18A-M and FIGS. 19A-I) exacerbated disease progression, while Lactobacillus gasseri and Prevotella melaninogenica treatments (LG and PM, respectively) showed disease-promoting effects in some, but not all, of the behavioral tests (FIGS. 17A-L). Indeed, RT levels positively correlated with ALS progression in SOD1-Tg mice (FIG. 18A), worsened upon administration motor functions as indicated by rotarod, grip test and neurological scores, as indicated by the pooled results of 4 independent treatments (N=20-40 mice per group, FIGS. 18B-D), albeit some variability noted between the independently analyzed repetitions (N=5-10 mice in each group of each repeat, FIGS. 19A-I). No histological differences in neuronal death rates (FIGS. 18E-F), but higher early-onset (day 100) atrophy using T2-weighted MRI scans (FIGS. 18G-M) were found in RT-treated SOD1-Tg mice compared to vehicle-treated ones. Of note, none of the tested 11 bacterial strains affected motor abilities in WT animals (FIGS. 17G-I for 9 tested bacterial strains, and FIGS. 18A-M and FIGS. 19A-I for RT). Taken together, these results suggest that multiple commensals might contribute to motor neuron degeneration in the SOD1-Tg ALS mouse model.

AM Colonization Ameliorates Murine ALS and Prolongs Survival

One of the differentially altered species in SOD1-Tg mice at our vivarium was Akkermansia muciniphila (AM), with both 16S rDNA (FIG. 11C) and shotgun metagenomic sequencing (FIG. 15B and FIG. 3A) demonstrating that it gradually reduced in its abundance as disease progressed in SOD1-Tg mice, as compared to stably high representation in the WT littermate microbiome. Decreased levels of AM 16S rDNA copies at our vivarium were validated in SOD1-Tg stool samples using AM-specific qPCR (FIG. 3B). Treatment of Abx pre-treated SOD1-Tg and WT mice with an anaerobically mono-cultured AM strain (BAA-835, O.D.=0.7, stationary phase), administered orally at 6 day-intervals for a total of 15 treatments was associated with improved motor function in AM-treated SOD1-Tg mice as quantified by the rotarod, grip and neurological scoring tests and assessed in pooled samples (N=34-62 mice per group, FIGS. 3C-E) or independently from 6 repeats (N=5-25 mice in each group of each repeat, FIGS. 17A-C and FIGS. 20A-O). This AM-mediated functional improvement was accompanied by a higher motor neuron survival in the AM-treated SOD1-Tg spinal cords, as compared to vehicle-treated Abx-pre-treated SOD1-Tg mice (FIGS. 3F-G, p=0.0041). Importantly, AM treatment significantly and substantially prolonged the life-span of SOD1-Tg mice compared to vehicle-treated mice or to SOD1-Tg mice treated with other commensal microbiome species serving as bacterial controls (FIG. 3H). AM treatment also reduced brain atrophy at day 140, as indicated by lower T2 relaxation time in specific ALS-affected brain areas measured by MRI (FIGS. 21A-D). The beneficial effect of AM on ALS progression did not result from altered gut permeability that may be induced by this bacterium in other contexts³², as no differences in systemic FITC-dextran influx were found at day 120 between AM-, PBS- and other microbial treated SOD1-Tg and WT mice (FIG. 21E). The microbiome metagenome of AM-treated SOD1-Tg mice clustered differently than that of PBS-treated SOD1-Tg controls (FIG. 21F). As expected, AM relative abundance was significantly increased in stool samples of AM-treated as compared to vehicle-treated SOD1-Tg mice (FIG. 21G). In contrast, WT mice harboring high and stable indigenous AM levels at our vivarium featured competitive exclusion of exogenously-administered AM whose levels rose only upon prolonged administration (FIG. 21H). Moreover, AM was found to colonize more broadly and efficiently in different regions of the SOD1-Tg GI tract comparing to the WT GI tract (FIGS. 21I-J). Consequently, AM supplementation following Abx treatment altered the microbiome composition of both WT and SOD1-Tg mice in distinct manners (FIGS. 21K, L).

To further validate our results, we mono-colonized Abx-pretreated SOD1-Tg and WT littermates with another strain of AM (ATCC 2869). Similar to the results observed with AM (ATCC BAA 835), AM 2869-colonized SOD1-Tg mice presented significant improvement in their motor abilities (FIGS. 22A-C) suggesting that the observed beneficial effect of AM on ALS symptoms may span different AM strains. Since AM is a mucin glycan degrading bacterium³³, we further conducted a histopathological analysis of distal colon mucus of AM- or PBS-treated SOD1-Tg at day 140. An intact inner mucus layer mucus was observed in AM supplemented and in PBS-treated SOD1-Tg mice (FIG. 23A). In contrast to PBS-treated control SOD1-Tg mice, the AM-treated SOD1-Tg mice had bacteria penetrated the inner mucus and in rare cases into the crypts (FIG. 23B, white arrows). A proteomic analysis did not feature significant differences in mucus components levels in AM-supplemented mice (FIGS. 23C-J). Collectively, assessment of multiple differentially expressed gut commensals by their mono-inoculation into SOD1-Tg mice identified selected commensals that adversely (PD, RT, and potentially LG and PM) or favorably (AM) modulate mouse-ALS disease course and severity.

AM Attenuates Murine ALS by Systemically Elevating Nicotinamide Levels

The above modulatory impacts of distinct gut commensals on murine ALS clinical course are likely contributed by a variety of mechanisms. As one example, we next assessed microbiome-induced mechanisms, potentially explaining the AM-mediated beneficial effects on mouse-ALS disease course at our vivarium. Given the remoteness of the gut microbiome from the CNS disease site, we hypothesized that intestinal microbiome-regulated metabolites may impact motor neuron susceptibility in SOD1-Tg mice by translocating to the CNS^{9,10}. To this aim, we utilized untargeted metabolomic profiling to identify candidate microbiome-dependent molecules differentially abundant in sera of AM-supplemented and vehicle controls, during the early stage of ALS (day 100). Out of 711 serum metabolites identified in SOD1-Tg mice, 84 metabolites were significantly altered by AM supplementation, out of which 51 were elevated by AM treatment (FIG. 4A and FIGS. 24A-C). Of these, the biosynthetic genes (nucleotide sequences, KEGG database) of only 6 metabolites were aligned to our metagenomic index, with two metabolites, Nicotinamide and Phenol sulfate, featuring the highest metagenomic probabilities to be synthesized by the WT microbiome over the SOD1-Tg microbiome at our vivarium (FIG. 24D). Administration of Phenol sulfate to SOD1-Tg mice, using subcutaneously implanted slow-release mini osmotic pumps ensuring continuous drug administration for the duration of murine ALS course, did not affect ALS symptoms in vivo (FIGS. 24E-G).

Several key observations suggested that NAM may be involved in AM-mediated murine-ALS positive modulation. Marked alterations in the metagenomic NAM biosynthetic pathway were noted upon Abx treatment (FIG. 2H). Enrichment in serum level of NAM biosynthetic intermediates was noted upon AM supplementation (FIG. 4B). Additionally, shotgun metagenomic sequencing revealed that several genes of the gut microbiome-derived tryptophan metabolizing pathway (FIGS. 2F-G), which has also been shown to be involved in generation of NAM³⁴³⁵, were substantially reduced in naïve SOD1 mice, while systemic metabolites of the tryptophan pathway were altered upon Abx treatment or AM supplementation (FIGS. 25A-B), suggesting that microbiome modulation of tryptophan metabolism could potentially contribute to altered NAM levels in these setting.

To examine whether AM is able to produce and secrete NAM, we measured NAM levels in anaerobically-grown AM and control gram positive and negative commensal isolates, using targeted metabolomics. Indeed, significantly higher levels of NAM were found in the medium of AM cultures, compared to supernatants collected from heat-killed AM or from other commensal isolates (FIG. 4C). To further explore the possibility that AM-secreted/induced-NAM may reach the CNS and affect motor neurons, we measured NAM levels in the CSF of AM-treated as compared to vehicle-treated SOD1-Tg and WT littermate mice at our vivarium. Indeed, CSF NAM levels were significantly higher in both AM-treated SOD1-Tg and WT mice already at age 100 days (early-stage disease) (FIG. 4D). During advanced stages of the disease (day 140), CSF NAM levels were significantly higher in AM-treated SOD1-Tg mice but not in AM-treated WT mice as compared to untreated controls (FIG. 4E), potentially reflecting gut colonization stability differences noted between WT and SOD1-Tg mice (FIGS. 21G-L). Importantly, 8 out of the 10 AM genome-related genes that encode enzymes participating in NAM metabolism, were significantly enriched in AM-treated SOD1-Tg mice compared to vehicle-treated SOD1-Tg mice (FIG. 4F), indicating that AM supplementation in SOD1-Tg mice may directly modify functional NAM biosynthesis.

To causally link increased systemic NAM levels to the associated phenotypic effects noted upon AM supplementation, we continuously supplemented SOD1-Tg mice with NAM, administered subcutaneously through implanted mini-osmotic pumps releasing NAM at a constant rate of 0.11 μl/hr and a cumulative dosage of 49.28 mg/kg/week. By replacing the pumps every 28 days, for a total of 4 times between the ages of 40-152 days, we assured steady and continuous NAM administration to mice throughout the disease. Indeed, NAM levels were significantly increased in the CSF and sera of NAM-treated SOD1-Tg mice compared to water-treated controls (FIGS. 5A-B). Importantly, NAM-treated SOD1-Tg mice performed significantly better than vehicle-treated SOD1-Tg mice, in both behavioral and neurological motor tests, as indicated by a pooled analysis (N=30 mice per group, FIGS. 5C-E) or independently in three repeats (N=10 mice in each group of each repeat, FIGS. 26A-I). Of note, NAM treatment resulted in a non-significantly trend to improve survival (FIG. 5F), possibly reflecting insufficient dosing or exposure time, or the necessity for integration of other AM-mediated modulatory mechanisms (FIG. 3H) in reaching the observed AM-induced survival benefit.

To examine whether NAM produced by GI bacteria is able to affect motor abilities, we inoculated Abx-pretreated SOD1-Tg mice with either WT E. coli as control or with the AnadA E. coli harboring compromised NAM production (FIG. 27). Of note, E. coli is considered a poor colonizer of the mouse GI tract³⁶. While ΔnadA E. coli supplementation did not affect rotarod and grip test performances (FIG. 27), it significantly improved the neurological scores of SOD1-Tg mice compared to the WT E. coli-treated animals (FIG. 5G), suggesting that NAM secreted from gut bacteria, even with poor colonization capacity, is able to impact some motor abilities in this ALS mouse model.

Potential AM and NAM Mechanisms of ALS Modulation

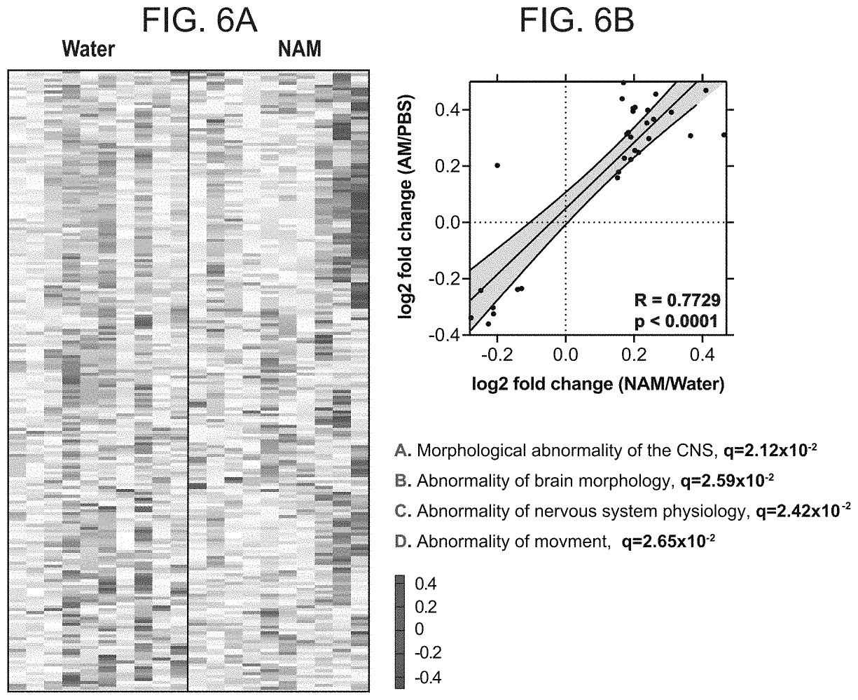

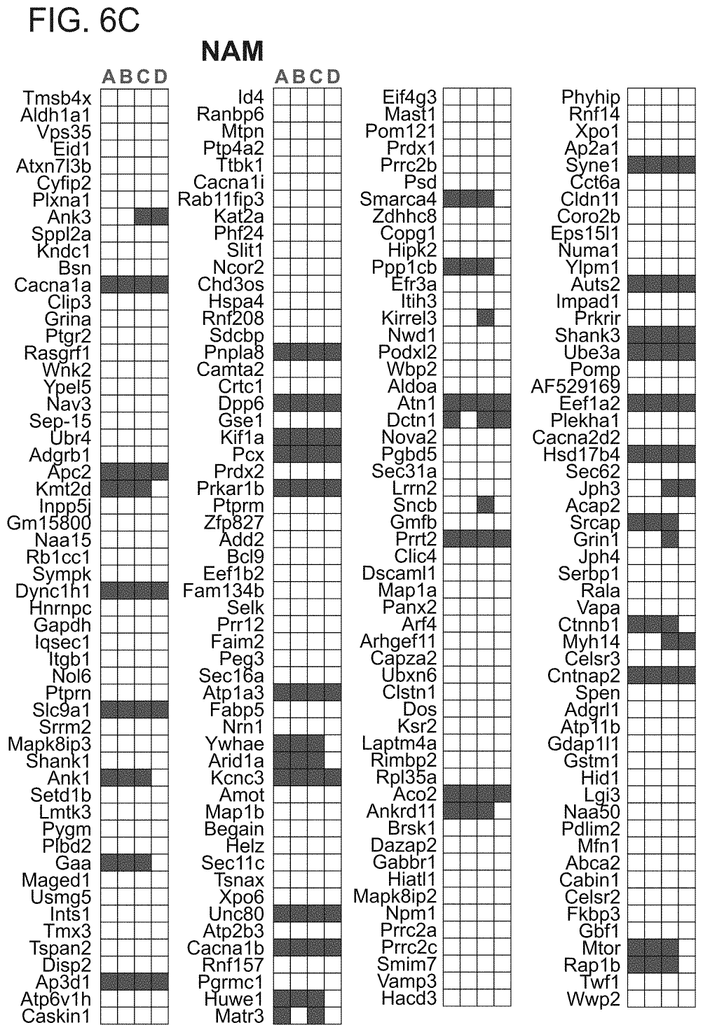

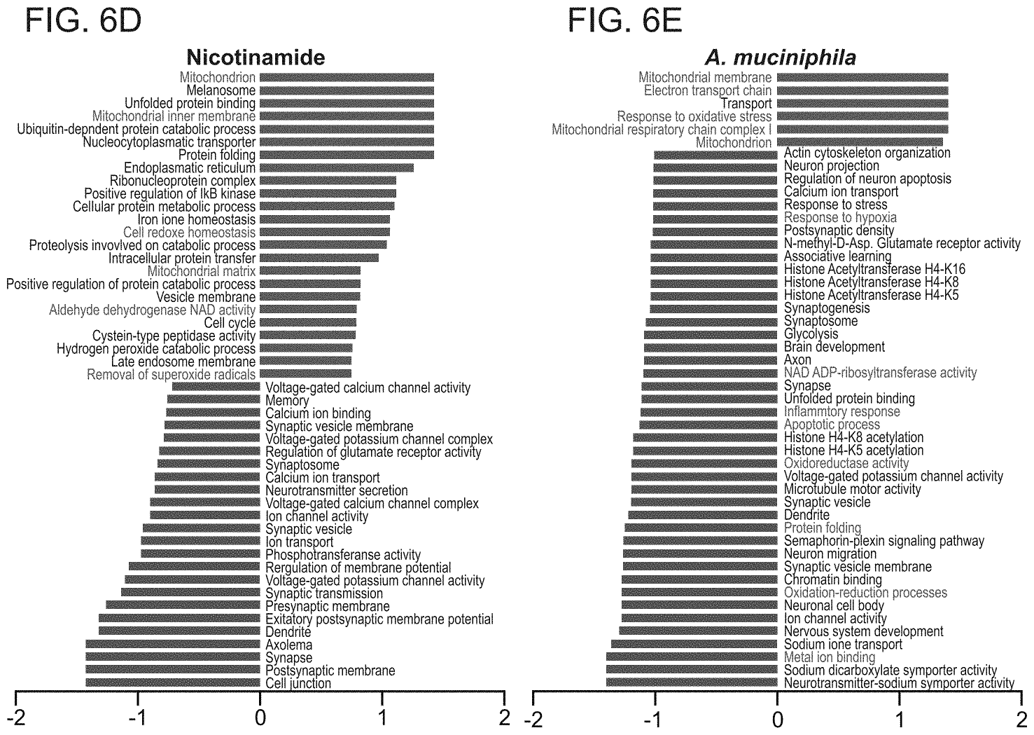

To explore potential molecular mechanisms by which AM and NAM may support motor neuron survival and ameliorate ALS progression in SOD1-Tg mice, we conducted bulk RNA-sequencing (RNA-seq) of spinal cord samples collected from AM- and NAM-treated mice at our vivarium and compared the transcriptional changes induced by AM- or NAM supplementation treatment, with their corresponding controls (PBS-treated or water-treated controls, in AM and NAM-treatment experiments, respectively). Overall, false discovery rate (FDR)-corrected expression of 213 genes significantly changed following NAM treatment of SOD1-Tg mice (FIG. 6A). 31 of these genes also significantly correlated in their expression pattern following AM treatment (FIG. 6B). Annotating the NAM-responsive genes to phenotype ontology resulted in a significant 21% fit to 4 categories related to abnormal brain morphology, physiology and movement, indicating that these genes may also be disease-modifying (FIG. 6C). To determine the functionality of AM- and NAM-affected transcripts, we assigned GO (Gene Ontology) pathways to each group of genes (FIGS. 6D-E). The most significantly enriched pathways shared between AM and NAM interventions are related to mitochondrial structure and function, Nicotinamide adenine dinucleotide⁺ (NAD⁺) homeostasis and removal of superoxide radicals, canonical functions known to be disrupted in ALS. Interestingly, 28.6% of the shared genes between AM and NAM treatments were found to be regulated by the transcription factor Nuclear Respiratory Factor-1 (NRF-1, FIG. 28), known to control mitochondrial biogenesis, electron transport chain activity and oxidative stress³⁷⁻⁴¹.

Dysbiosis & Impaired NAM Levels in Human ALS Patients

Finally, we examined preliminary links between the SOD1-Tg findings at our vivarium and features of human ALS. To this aim, we performed a human observational study, by collecting stool samples from 32 ALS patients and 27 healthy BMI- and Aged-matched family members as controls and sequencing their gut microbiome metagenomes. The microbiome composition of ALS patients, as quantified by shotgun metagenomic sequencing, was significantly different to that of healthy control household members (FIG. 7A, for PC1: p=3.3×10⁻⁶). While we did not observe any significant difference in specific bacterial species abundances after FDR correction, multiple compositional trends could be noted (FIG. 29A), potentially implying that the significantly distinct global clustering of human ALS microbiomes stemmed from numerous accumulated small changes in bacterial abundances. Functionally, ALS microbiomes showed a significant difference in the global bacterial gene content (FIG. 7B, for PC1: p=2.88×10⁻⁹), accompanied by FDR-corrected (adjusted for these pathways) decrease in several key genes participating in tryptophan and in NAM metabolism, such as Purine nucleoside phosphorylase (K03783, punA), Nicotinamide-nucleotide amidase (K03742, Amuc_0430), L-aspartate oxidase (K00278, Amuc_1079) NAD synthase (K01950, Amuc_0620), 2-oxoglutarate dehydrogenase (K00164, OGDH), Nicotinate-nucleotide pyrophosphorylase (K00767, Amuc_1263) and Enoyl-CoA hydratase (K01782, fadJ, FIG. 7C). Importantly, some of these significantly reduced genes were all mapped to the A. muciniphila genome, suggesting that, while the relative abundance of AM in the microbiome of the examined ALS patients was similar to that of healthy controls, the NAM-biosynthesis capacity of distinct AM strains could be differentially impaired in ALS.

Untargeted metabolomic profiling of sera of ALS patients revealed multiple significantly-changed metabolites, including elevated riluzole (an ALS exogenously administrated treatment), creatine and 3-hydroxy-2-ethylpropionate and reduced methyl indole 3-acetate and triethanolamine (FIG. 29B). Interestingly, key molecules of the tryptophan-nicotinamide metabolic pathway were significantly altered in the sera of ALS patients, among them Indoleacetate, Kynurenine, Serotonin and circulating Nicotinamide (FIGS. 7D-E), suggesting an aberrant NAM metabolism in some of these human ALS cases. To examine whether these systemic aberrations may also be reflected at the CNS, we compared the levels of NAM in the CSF of 12 ALS patients with that of 17 healthy non-household controls. Average NAM CSF levels of ALS patients were significantly lower than those of healthy individuals, with some patients featuring markedly low NAM CSF levels (FIG. 7F).

### REFERENCES

1. Turner, M. R. et al. Controversies and priorities in amyotrophic lateral sclerosis. The Lancet Neurology 12, 310-322 (2013).
2. Andersen, P. M. & Al-Chalabi, A. Clinical genetics of amyotrophic lateral sclerosis: What do we really know? Nature Reviews Neurology (2011). doi:10.1038/nrneurol.2011.150
3. Bensimon, G., Lacomblez, L. & Meininger, V. A Controlled Trial of Riluzole in Amyotrophic Lateral Sclerosis. N. Engl. J. Med. 330, 585-591 (1994).
4. Group, T. W. G. on behalf of the E. (MCI-186) A. 19 S. Safety and efficacy of edaravone in well defined patients with amyotrophic lateral sclerosis: a randomised, double-blind, placebo-controlled trial. Lancet Neurol. 16, 505-512 (2017).
5. Al-Chalabi, A. & Hardiman, 0. The epidemiology of ALS: A conspiracy of genes, environment and time. Nature Reviews Neurology 9, 617-628 (2013).
6. Veiga-fernandes, H. & Mucida, D. Review Neuro-Immune Interactions at Barrier Surfaces. Cell 165, 801-811 (2016).
7. Sharon, G., Sampson, T. R., Geschwind, D. H. & Mazmanian, S. K. The Central Nervous System and the Gut Microbiome. Cell 167, 915-932 (2016).
8. Blacher, E., Levy, M., Tatirovsky, E. & Elinav, E. Microbiome-Modulated Metabolites at the Interface of Host Immunity. J. Immunol. 198, 572-580 (2017).
9. Hsiao, E. Y. et al. Microbiota modulate behavioral and physiological abnormalities associated with neurodevelopmental disorders. Cell 155, 1451-1463 (2013).
10. Sampson, T. R. et al. Gut Microbiota Regulate Motor Deficits and Neuroinflammation in a Model of Parkinson's Disease. Cell 167, 1469-1480.e12 (2016).
11. Harach, T. et al. Reduction of Abeta amyloid pathology in APPPS1 transgenic mice in the absence of gut microbiota. Sci. Rep. 7, 41802 (2017).
12. Cignarella, F. et al. Intermittent Fasting Confers Protection in CNS Autoimmunity by Altering the Gut Microbiota. Cell Metab. 27, 1222-1235 (2018).
13. Yadav, S. K. et al. Gut dysbiosis breaks immunological tolerance toward the central nervous system during young adulthood. Proc. Natl. Acad. Sci. 144, 9318-9327 (2017).
14. Berer, K. et al. Gut microbiota from multiple sclerosis patients enables spontaneous autoimmune encephalomyelitis in mice. Proc. Natl. Acad. Sci. 114, 10719-10724 (2017).
15. Cekanaviciute, E. et al. Gut bacteria from multiple sclerosis patients modulate human T cells and exacerbate symptoms in mouse models. Proc. Natl. Acad. Sci. 114, 10713-10718 (2017).
16. Olson, C. A. et al. The Gut Microbiota Mediates the Anti-Seizure Effects of the Ketogenic Diet. Cell 173, 1728-1741 (2018).
17. Rowin, J., Xia, Y., Jung, B. & Sun, J. Gut inflammation and dysbiosis in human motor neuron disease. Physiol. Rep. 5, 1-6 (2017).
18. Fang, X. Potential role of gut microbiota and tissue barriers in Parkinson's disease and amyotrophic lateral sclerosis. Int. J. Neurosci. 126, 771-776 (2016).
19. Alonso, R. et al. Evidence for fungal infection in cerebrospinal fluid and brain tissue from patients with amyotrophic lateral sclerosis. Int. J. Biol. Sci. 11, 546-558 (2015).
20. Wu, S., Yi, J., Zhang, Y. G., Zhou, J. & Sun, J. Leaky intestine and impaired microbiome in an amyotrophic lateral sclerosis mouse model. Physiol. Rep. 3, e12356 (2015).
21. Zhang, Y. guo et al. Target Intestinal Microbiota to Alleviate Disease Progression in Amyotrophic Lateral Sclerosis. Clin. Ther. 39, 322-336 (2017).
22. Fang, X. et al. Evaluation of the microbial diversity in amyotrophic lateral sclerosis using high-throughput sequencing. Front. Microbiol. 7, 1474 (2016).
23. Brenner, D. et al. The fecal microbiome of ALS patients. Neurobiol. Aging 61, 132-137 (2018).
24. Gurney, M. E. et al. Motor neuron degeneration in mice that express a human Cu,Zn superoxide dismutase mutation. Science (80-.). 264, 1772-1775 (1994).
25. Hill, D. A. et al. Metagenomic analyses reveal antibiotic-induced temporal and spatial changes in intestinal microbiota with associated alterations in immune cell homeostasis. Mucosal Immunol. (2010). doi:10.1038/mi.2009.132
26. Ralph, G. S. et al. Silencing mutant SOD1 using RNAi protects against neurodegeneration and extends survival in an ALS model. Nat. Med. 11, 429-433 (2005).
27. Aartsma-Rus, A. & van Putten, M. Assessing functional performance in the mdx mouse model. J. Vis. Exp. 27, 51303 (2014).
28. Sugiyama, K. & Tanaka, K. Spinal cord-specific deletion of the glutamate transporter GLT1 causes motor neuron death in mice. Biochem. Biophys. Res. Commun. 497, 689-693 (2018).
29. Bontempi, P. et al. MRI reveals therapeutical efficacy of stem cells: An experimental study on the SOD1(G93A) animal model. Magn. Reson. Med. 79, 459-469 (2018).
30. Niessen, H. G. et al. In vivo quantification of spinal and bulbar motor neuron degeneration in the G93A-SOD1 transgenic mouse model of ALS by T2 relaxation time and apparent diffusion coefficient. Exp. Neurol. 201, 293-300 (2006).
31. Zang, D. W. et al. Magnetic resonance imaging reveals neuronal degeneration in the brainstem of the superoxide dismutase 1 transgenic mouse model of amyotrophic lateral sclerosis. Eur. J. Neurosci. 20, 1745-51 (2004).
32. Chelakkot, C. et al. Akkermansia muciniphila-derived extracellular vesicles influence gut permeability through the regulation of tight junctions. Exp. Mol. Med. 50, e450 (2018).
33. Derrien, M., Vaughan, E. E., Plugge, C. M. & de Vos, W. M Akkermansia municiphila gen. nov., sp. nov., a human intestinal mucin-degrading bacterium. Int. J. Syst. Evol. Microbiol. (2004). doi:10.1099/ijs.0.02873-0
34. Cantó, C., Menzies, K. J. & Auwerx, J. NAD+ Metabolism and the Control of Energy Homeostasis: A Balancing Act between Mitochondria and the Nucleus. Cell Metabolism 22, 31-53 (2015).
35. Verdin, E. NAD+in aging, metabolism, and neurodegeneration. Science 350, 1208-1213 (2015).
36. Sweeney, N. J., Laux, D. C. & Cohen, P. S. Escherichia coli F-18 and E. coli K-12 eda mutants do not colonize the streptomycin-treated mouse large intestine. Infect. Immun. (1996).
37. Golko-Perez, S., Amit, T., Bar-Am, O., Youdim, M. B. H. & Weinreb, O. A Novel Iron Chelator-Radical Scavenger Ameliorates Motor Dysfunction and Improves Life Span and Mitochondrial Biogenesis in SOD1G93AALS Mice. Neurotox. Res. 31, 230-244 (2017).
38. Scarpulla, R. C., Vega, R. B. & Kelly, D. P. Transcriptional integration of mitochondrial biogenesis. Trends in Endocrinology and Metabolism 23, 459-466 (2012).
39. Bergeron, R. et al. Chronic activation of AMP kinase results in NRF-1 activation and mitochondrial biogenesis. Am. J. Physiol. Endocrinol. Metab. 281, 1340-1346 (2001).
40. Evans, M. J. & Scarpulla, R. C. NRF-1: A trans-activator of nuclear-encoded respiratory genes in animal cells. Genes Dev. 4, 1023-1034 (1990).
41. Virbasius, C. M. A., Virbasius, J. V. & Scarpulla, R. C. NRF-1, an activator involved in nuclear-mitochondrial interactions, utilizes a new DNA-binding domain conserved in a family of developmental regulators. Genes Dev. 7, 2431-2445 (1993).
42. Sampson, T. R. & Mazmanian, S. K. Control of brain development, function, and behavior by the microbiome. Cell Host and Microbe 17, 565-576 (2015).
43. Collins, S. M., Surette, M. & Bercik, P. The interplay between the intestinal microbiota and the brain. Nat. Rev. Microbiol. 10, 735-742 (2012).
44. Yano, J. M. et al. Indigenous bacteria from the gut microbiota regulate host serotonin biosynthesis. Cell 161, 264-276 (2015).
45. Pessione, E. et al. A proteomic approach to studying biogenic amine producing lactic acid bacteria. in Proteomics 5, 687-698 (2005).
46. Levy, M. et al. Microbiota-Modulated Metabolites Shape the Intestinal Microenvironment by Regulating NLRP6 Inflammasome Signaling. Cell 163, 1428-1443 (2015).
47. Pessione, E. et al. First evidence of a membrane-bound, tyramine and β-phenylethylamine producing, tyrosine decarboxylase in Enterococcus faecalis: A two-dimensional electrophoresis proteomic study. Proteomics 9, 2695-2710 (2009).
48. Tanous, C., Chambellon, E., Sepulchre, A. M. & Yvon, M. The gene encoding the glutamate dehydrogenase in Lactococcus lactis is part of a remnant Tn3 transposon carried by a large plasmid. J. Bacteriol. 187, 5019-5022 (2005).
49. Zareian, M. et al. A glutamic acid-producing lactic acid bacteria isolated from malaysian fermented foods. Int. J. Mol. Sci. 13, 5482-5497 (2012).
50. Han, T. C., Kwon, D. H., Hegazy, M. & Lu, C. D. Transcriptome analysis of agmatine and putrescine catabolism in Pseudomonas aeruginosa PAO1. J. Bacteriol. 190, 1966-1975 (2008).
51. Mazzoli, R. et al. Glutamate-induced metabolic changes in Lactococcus lactis NCDO 2118 during GABA production: Combined transcriptomic and proteomic analysis. Amino Acids 39, 727-737 (2010).
52. Wong, R. K., Yang, C., Song, G. H., Wong, J. & Ho, K. Y. Melatonin Regulation as a Possible Mechanism for Probiotic (VSL #3) in Irritable Bowel Syndrome: A Randomized Double-Blinded Placebo Study. Dig. Dis. Sci. 60, 186-194 (2014).
53. Benakis, C. et al. Commensal microbiota affects ischemic stroke outcome by regulating intestinal γδ T cells. Nat. Med. 22, 516-523 (2016).
54. Lee, Y. K., Menezes, J. S., Umesaki, Y. & Mazmanian, S. K. Proinflammatory T-cell responses to gut microbiota promote experimental autoimmune encephalomyelitis. Proc. Natl. Acad. Sci. 108, 4615-4622 (2011).
55. Veit Rothhammer, Ivan D Mascanfroni, L. B. . . . et. Type 1 Interferons and microbial metabolites of tryptophan modulate astrocyte activity and inflammation via the aryl hydrocarbon receptor. Nat. Med. 22, 586-597 (2016).
56. Chini, E. N., Chini, C. C. S., Espindola Netto, J. M., de Oliveira, G. C. & van Schooten, W. The Pharmacology of CD38/NADase: An Emerging Target in Cancer and Diseases of Aging. Trends in Pharmacological Sciences 39, 424-436 (2018).
57. Tarragó, M. G. et al. A Potent and Specific CD38 Inhibitor Ameliorates Age-Related Metabolic Dysfunction by Reversing Tissue NAD+Decline. Cell Metab. 27, 1081-1095 (2018).
58. Camacho-Pereira, J. et al. CD38 Dictates Age-Related NAD Decline and Mitochondrial Dysfunction through an SIRT3-Dependent Mechanism. Cell Metab. 23, 1127-1139 (2016).
59. Ryu, D. et al. NAD+repletion improves muscle function in muscular dystrophy and counters global parylation. Sci. Transl. Med. 8, 361ra139 (2016).
60. Chang, W.-T., Chen, H., Chiou, R.-J., Chen, C.-Y. & Huang, A.-M. A novel function of transcription factor alpha-Pal/NRF-1: increasing neurite outgrowth. Biochem. Biophys. Res. Commun. 334, 199-206 (2005).
61. Li, Z., Cogswell, M., Hixson, K., Brooks-Kayal, A. R. & Russek, S. J. Nuclear Respiratory Factor 1 (NRF-1) Controls the Activity Dependent Transcription of the GABA-A Receptor Beta 1 Subunit Gene in Neurons. bioRxiv 321257, 1-25 (2018).
62. Robertson, S. J. et al. Nod1 and Nod2 signaling does not alter the composition of intestinal bacterial communities at homeostasis. Gut Microbes 4, 222-231 (2013).
63. Korem, Tal; Zeevi, David; Zmora, Niv; Weissbrod, Omer; Bar, Noam; Lotan-Pompan, Maya; Avnit-Sagi, Tali; Kosower, Noa; Malka, Gal; Rein, Michal; Suez, Jotham; Goldberg, Ben Z.; Weinberger, Adina; Levy, Avraham A.; Elinav, Eran; and Segal, E. Bread Affects Clinical Parameters and Induces Gut Microbiome-Associated Personal Glycemic Responses. Cell Metab. 25, 1243-1253 (2017).
64. Zeevi, D. et al. Personalized nutrition by prediction of glycemic responses. Cell 163, 1079-1095 (2015).
65. Hatzipetros, T. et al. A Quick Phenotypic Neurological Scoring System for Evaluating Disease Progression in the SOD1-G93A Mouse Model of ALS. J. Vis. Exp. 104, 1-6 (2015).
66. Hansson, G. C. & Johansson, M. E. V. The inner of the two Muc2 mucin-dependent mucus layers in colon is devoid of bacteria. Gut Microbes (2010). doi:10.4161/gmic.1.1.10470
67. Wiśniewski, J. R., Zougman, A., Nagaraj, N. & Mann, M. Universal sample preparation method for proteome analysis. Nat. Methods (2009). doi:10.1038/nmeth.1322
68. Schroeder, B. O. et al. Bifidobacteria or Fiber Protects against Diet-Induced Microbiota-Mediated Colonic Mucus Deterioration. Cell Host Microbe (2018). doi:10.1016/j.chom.2017.11.004
69. Rappsilber, J., Mann, M. & Ishihama, Y. Protocol for micro-purification, enrichment, pre-fractionation and storage of peptides for proteomics using StageTips. Nat. Protoc. (2007). doi:10.1038/nprot.2007.261
70. Cox, J. & Mann, M. MaxQuant enables high peptide identification rates, individualized p.p.b.-range mass accuracies and proteome-wide protein quantification. Nat. Biotechnol. (2008). doi:10.1038/nbt.1511
71. MacLean, B. et al. Skyline: An open source document editor for creating and analyzing targeted proteomics experiments. Bioinformatics (2010). doi:10.1093/bioinformatics/btq054
72. Baba, T. et al. Construction of Escherichia coli K-12 in-frame, single-gene knockout mutants: The Keio collection. Mol. Syst. Biol. (2006). doi:10.1038/msb4100050
73. Schneeberger, M. et al. Akkermansia muciniphila inversely correlates with the onset of inflammation, altered adipose tissue metabolism and metabolic disorders during obesity in mice. Sci. Rep. 5, 16643 (2015).
74. Suez, J. et al. Artificial sweeteners induce glucose intolerance by altering the gut microbiota. Nature (2014). doi:10.1038/nature13793
75. Adiconis, X. et al. Comparative analysis of RNA sequencing methods for degraded or low-input samples. Nat. Methods 10, 623-629 (2013).
76. Caporaso, J. G., Kuczynski, J. & et al. QIIME allows high throughput community sequencing data. Nat. Methods 7, 335-336 (2010).
77. Callahan, B. J. et al. DADA2: High-resolution sample inference from Illumina amplicon data. Nat. Methods 13, 581-583 (2016).
78. Weiss, S. et al. Normalization and microbial differential abundance strategies depend upon data characteristics. Microbiome 5, 27 (2017).
79. McDonald, D. et al. An improved Greengenes taxonomy with explicit ranks for ecological and evolutionary analyses of bacteria and archaea. ISME J. 6, 610-618 (2012).
80. Marco-Sola, S., Sammeth, M., Guigó, R. & Ribeca, P. The GEM mapper: Fast, accurate and versatile alignment by filtration. Nat. Methods 9, 1185-1188 (2012).
81. Loh, P. R. et al. Reference-based phasing using the Haplotype Reference Consortium panel. Nat. Genet. 48, 1443-1448 (2016).
82. Kanehisa, M. et al. From genomics to chemical genomics: new developments in KEGG. Nucleic Acids Res. 34, 354-357 (2006).
83. Buchfink, B., Xie, C. & Huson, D. H. Fast and sensitive protein alignment using DIAMOND. Nat. Methods 12, 59-60 (2015).
84. Langmead, B. & Salzberg, S. L. Fast gapped-read alignment with Bowtie 2. Nat. Methods (2012). doi:10.1038/nmeth.1923
85. Reimand, J., Kull, M., Peterson, H., Hansen, J. & Vilo, J. G:Profiler-a web-based toolset for functional profiling of gene lists from large-scale experiments. Nucleic Acids Res. (2007). doi:10.1093/nar/gkm226

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of treating ALS in a subject in need thereof comprising administering to the subject a therapeutically effective amount of at least two metabolites, wherein at least one of said at least two metabolites is selected from the group consisting of propyl 4-hydroxybenzoate, triethanolamine, serotonin, 2-keto-3-deoxy-gluconate, nicotinamide, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE thereby treating ALS.

2. (canceled)

3. The method of claim 1, wherein at least one of said at least two metabolites is selected from the group consisting of nicotinamide, phenol sulfate, equol sulfate and cinnamate.

4. The method of claim 1, wherein at least one of said at least two metabolites is selected from the group consisting of propyl 4-hydroxybenzoate, triethanolamine, serotonin, 2-keto-3-deoxy-gluconate nicotinamide, N-trimethyl 5-aminovalerate, phenylalanylglycine, theobromine, cys-gly, glutamate and 1-palmitoyl-2-docosahexaenoyl-GPC.

5. The method of claim 1, wherein said at least two metabolites are selected from the group consisting of propyl 4-hydroxybenzoate, triethanolamine, serotonin, 2-keto-3-deoxy-gluconate nicotinamide, N-trimethyl 5-aminovalerate, phenylalanylglycine, theobromine, cys-gly, glutamate and 1-palmitoyl-2-docosahexaenoyl-GPC.

6. The method of claim 1, wherein at least one of said at least two metabolites is nicotinamide.

7. The method of claim 1, wherein at least one of said at least two metabolites is comprised in a bacterial population.

8. The method of claim 7, wherein said bacterial population is selected from the group consisting of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, Akkermansia Muciniphila (AM), Anaeroplasma, Prevotella, Distanosis, Parabacteroides, Rikenellaceae, Alistipes, Candidatus Arthromitus, Eggerthella, Oscillibacter, Subdoligranulum and Lactobacillus.

9. A method of treating ALS in a subject in need thereof comprising administering to the subject a therapeutically effective amount of a probiotic comprising a bacterial population selected from the group consisting of Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum, Anaerostipes hadrus, Akkermansia Muciniphila (AM), Anaeroplasma, Prevotella, Distanosis, Parabacteroides, Rikenellaceae, Alistipes, Candidatus Arthromitus, Eggerthella, Oscillibacter, Subdoligranulum and Lactobacillus, thereby treating ALS.

10. The method of claim 8, wherein said bacterial population comprises Akkermansia Muciniphila (AM).

11. The method of claim 9, wherein said bacterial population comprises Akkermansia Muciniphila (AM).

12. The method of claim 9, wherein said bacterial population comprises Streptococcus thermophiles, Faecalibacterium prausnitzii, Eubacterium rectale, Bacteroides plebeius, Coprococcus, Roseburia hominis, Eubacterium ventriosum, Lachnospiraceae, Eubacterium hallii, Bacteroidales, Bifidobacterium pseudocatenulatum and Anaerostipes hadrus.

13-20. (canceled)

21. A method of treating ALS in a subject in need thereof comprising administering to the subject a therapeutically effective amount of a metabolite selected from the group consisting of propyl 4-hydroxybenzoate, triethanolamine, serotonin, 2-keto-3-deoxy-gluconate, N-trimethyl 5-aminovalerate, phenylalanylglycine, theobromine, cys-gly, glutamate, 1-palmitoyl-2-docosahexaenoyl-GPC, oxalate, stearoyl sphingomyelin, 1-palmitoyl-2-docosahexaenoyl-GPC (16:0/22:6), 3-ureidopropionate, 1-(1-enyl-palmitoyl)-2-arachidonoyl-GPC (P-16:0/20:4), palmitoyl sphingomyelin (d18:1/16:0), sphingomyelin (d18:1/18:1, d18:2/18:0), pyruvate, taurocholate, N-acetyltyrosine, tauro-beta-muricholate, tauroursodeoxycholate, phenol sulfate, equol sulfate, cinnamate, phenylpropionylglycine, 2-aminophenol sulfate, 4-allylphenol sulfate, equol glucuronide, palmitoleoyl-linoleoyl-glycerol, oleoyl-linolenoyl-glycerol, 1-palmitoyl-2-oleoyl-GPE, hydroquinone sulfate, guaiacol sulfate, diacylglycerol, palmitoyl-linoleoyl-glycerol, gentisate and 13-HODE+9-HODE thereby treating ALS.

22-32. (canceled)


SEQUENCE LISTING (104 sequences)

SEQ ID NO: 1 - DNA, 20 bp, Artificial sequence
  Single strand DNA oligonucleotide
cagcacgtgaaggtggggac

SEQ ID NO: 2 - DNA, 20 bp, Artificial sequence
  Single strand DNA oligonucleotide
ccttgcggttggcttcagat

SEQ ID NO: 3 - DNA, 19 bp, Artificial sequence
  Single strand DNA oligonucleotide
caacgcgmaraaccttacc

SEQ ID NO: 4 - DNA, 19 bp, Artificial sequence
  Single strand DNA oligonucleotide
  misc_feature (9)..(9): n is a, c, g, or t
ctaaccgangaacctyacc

SEQ ID NO: 5 - DNA, 19 bp, Artificial sequence
  Single strand DNA oligonucleotide
atacgcgargaaccttacc

SEQ ID NO: 6 - DNA, 19 bp, Artificial sequence
  Single strand DNA oligonucleotide
  misc_feature (2)..(2): n is a, c, g, or t
  misc_feature (18)..(18): n is a, c, g, or t
cnacgcgaagaaccttanc

SEQ ID NO: 7 - DNA, 19 bp, Artificial sequence
  Single strand DNA oligonucleotide
  misc_feature (14)..(14): n is a, c, g, or t
cgacrrccatgcancacct

SEQ ID NO: 8 - DNA, 31 bp, Artificial sequence
  Single strand DNA oligonucleotide
tatggtaattgtgtgccagcmgccgcggtaa

SEQ ID NO: 9 - DNA, 32 bp, Artificial sequence
  Single strand DNA oligonucleotide
agtcagtcagccggactachvgggtwtctaat

SEQ ID NO: 10 - DNA, 39 bp, Artificial sequence
  Single strand DNA oligonucleotide
attagawacccbdgtagtccggctgactgactattagaa

SEQ ID NO: 11 - DNA, 50 bp, Artificial sequence
  Single strand DNA oligonucleotide
taatgatccttccgcaggttcacctacggaaaccttgttacgacttttac

SEQ ID NO: 12 - DNA, 50 bp, Artificial sequence
  Single strand DNA oligonucleotide
ttcctctagatagtcaagttcgaccgtcttctcagcgctccgccagggcc

SEQ ID NO: 13 - DNA, 50 bp, Artificial sequence
  Single strand DNA oligonucleotide
gtgggccgaccccggcggggccgatccgagggcctcactaaaccatccaa

SEQ ID NO: 14 - DNA, 50 bp, Artificial sequence
  Single strand DNA oligonucleotide
tcggtagtagcgacgggcggtgtgtacaaagggcagggacttaatcaacg

SEQ ID NO: 15 - DNA, 50 bp, Artificial sequence
  Single strand DNA oligonucleotide
caagcttatgacccgcacttactcgggaattccctcgttcatggggaata

SEQ ID NO: 16 - DNA, 50 bp, Artificial sequence
  Single strand DNA oligonucleotide
attgcaatccccgatccccatcacgaatggggttcaacgggttacccgcg

SEQ ID NO: 17 - DNA, 50 bp, Artificial sequence
  Single strand DNA oligonucleotide
cctgccggcgtagggtaggcacacgctgagccagtcagtgtagcgcgcgt

SEQ ID NO: 18 - DNA, 50 bp, Artificial sequence
  Single strand DNA oligonucleotide
gcagccccggacatctaagggcatcacagacctgttattgctcaatctcg

SEQ ID NO: 19 - DNA, 50 bp, Artificial sequence
  Single strand DNA oligonucleotide
ggtggctgaacgccacttgtccctctaagaagttgggggacgccgaccgc

SEQ ID NO: 20 - DNA, 50 bp, Artificial sequence
  Single strand DNA oligonucleotide
tcgggggtcgcgtaactagttagcatgccagagtctcgttcgttatcgga

SEQ ID NO: 21 - DNA, 50 bp, Artificial sequence
  Single strand DNA oligonucleotide
attaaccagacaaatcgctccaccaactaagaacggccatgcaccaccac

SEQ ID NO: 22 - DNA, 50 bp, Artificial sequence
  Single strand DNA oligonucleotide
ccacggaatcgagaaagagctatcaatctgtcaatcctgtccgtgtccgg

SEQ ID NO: 23 - DNA, 50 bp, Artificial sequence
  Single strand DNA oligonucleotide
gccgggtgaggtttcccgtgttgagtcaaattaagccgcaggctccactc

SEQ ID NO: 24 - DNA, 50 bp, Artificial sequence
  Single strand DNA oligonucleotide
ctggtggtgcccttccgtcaattcctttaagtttcagctttgcaaccata

SEQ ID NO: 25 - DNA, 50 bp, Artificial sequence
  Single strand DNA oligonucleotide
ctccccccggaacccaaagactttggtttcccggaagctgcccggcgggt

SEQ ID NO: 26 - DNA, 50 bp, Artificial sequence
  Single strand DNA oligonucleotide
catgggaataacgccgccgcatcgccggtcggcatcgtttatggtcggaa

SEQ ID NO: 27 - DNA, 50 bp, Artificial sequence
  Single strand DNA oligonucleotide
ctacgacggtatctgatcgtcttcgaacctccgactttcgttcttgatta

SEQ ID NO: 28 - DNA, 50 bp, Artificial sequence
  Single strand DNA oligonucleotide
atgaaaacattcttggcaaatgctttcgctctggtccgtcttgcgccggt

SEQ ID NO: 29 - DNA, 50 bp, Artificial sequence
  Single strand DNA oligonucleotide
ccaagaatttcacctctagcggcgcaatacgaatgcccccggccgtccct

SEQ ID NO: 30 - DNA, 50 bp, Artificial sequence
  Single strand DNA oligonucleotide
cttaatcatggcctcagttccgaaaaccaacaaaatagaaccgcggtcct

SEQ ID NO: 31 - DNA, 50 bp, Artificial sequence
  Single strand DNA oligonucleotide
attccattattcctagctgcggtatccaggcggctcgggcctgctttgaa

SEQ ID NO: 32 - DNA, 50 bp, Artificial sequence
  Single strand DNA oligonucleotide
cactctaattttttcaaagtaaacgcttcgggccccgcgggacactcagc

SEQ ID NO: 33 - DNA, 50 bp, Artificial sequence
  Single strand DNA oligonucleotide
taagagcatcgagggggcgccgagaggcaaggggcggggacgggcggtgg

SEQ ID NO: 34 - DNA, 50 bp, Artificial sequence
  Single strand DNA oligonucleotide
ctcgcctcgcggcggaccgcccgcccgctcccaagatccaactacgagct

SEQ ID NO: 35 - DNA, 50 bp, Artificial sequence
  Single strand DNA oligonucleotide
ttttaactgcagcaactttaatatacgctattggagctggaattaccgcg

SEQ ID NO: 36 - DNA, 50 bp, Artificial sequence
  Single strand DNA oligonucleotide
gctgctggcaccagacttgccctccaatggatcctcgttaaaggatttaa

SEQ ID NO: 37 - DNA, 50 bp, Artificial sequence
  Single strand DNA oligonucleotide
agtggactcattccaattacagggcctcgaaagagtcctgtattgttatt

SEQ ID NO: 38 - DNA, 50 bp, Artificial sequence
  Single strand DNA oligonucleotide
tttcgtcactacctccccgggtcgggagtgggtaatttgcgcgcctgctg

SEQ ID NO: 39 - DNA, 50 bp, Artificial sequence
  Single strand DNA oligonucleotide
ccttccttggatgtggtagccgtttctcaggctccctctccggaatcgaa

SEQ ID NO: 40 - DNA, 50 bp, Artificial sequence
  Single strand DNA oligonucleotide
ccctgattccccgtcacccgtggtcaccatggtaggcacggcgactacca

SEQ ID NO: 41 - DNA, 48 bp, Artificial sequence
  Single strand DNA oligonucleotide
tcgaaagttgatagggcagacgttcgaatgggtcgtcgccgccacggg

SEQ ID NO: 42 - DNA, 50 bp, Artificial sequence
  Single strand DNA oligonucleotide
gcgtgcgatcggcccgaggttatctagagtcaccaaagccgccggcgccc

SEQ ID NO: 43 - DNA, 50 bp, Artificial sequence
  Single strand DNA oligonucleotide
gccccccggccggggccggagaggggctgaccgggttggttttgatctga

SEQ ID NO: 44 - DNA, 50 bp, Artificial sequence
  Single strand DNA oligonucleotide
taaatgcacgcatcccccccgcgaagggggtcagcgcccgtcggcatgta

SEQ ID NO: 45 - DNA, 50 bp, Artificial sequence
  Single strand DNA oligonucleotide
ttagctctagaattaccacagttatccaagtaggagaggagcgagcgacc

SEQ ID NO: 46 - DNA, 50 bp, Artificial sequence
  Single strand DNA oligonucleotide
aaaggaaccataactgatttaatgagccattcgcagtttcactgtaccgg

SEQ ID NO: 47 - DNA, 50 bp, Artificial sequence
  Single strand DNA oligonucleotide
ccgtgcgtacttagacatgcatggcttaatctttgagacaagcatatgct

SEQ ID NO: 48 - DNA, 50 bp, Artificial sequence
  Single strand DNA oligonucleotide
tggcttaatctttgagacaagcatatgctactggcaggatcaaccaggta

SEQ ID NO: 49 - DNA, 50 bp, Artificial sequence
  Single strand DNA oligonucleotide
aagcgacgctcagacaggcgtagccccgggaggaacccggggccgcaagt

SEQ ID NO: 50 - DNA, 50 bp, Artificial sequence
  Single strand DNA oligonucleotide
gcgttcgaagtgtcgatgatcaatgtgtcctgcaattcacattaattctc

SEQ ID NO: 51 - DNA, 50 bp, Artificial sequence
  Single strand DNA oligonucleotide
gcagctagctgcgttcttcatcgacgcacgagccgagtgatccaccgcta

SEQ ID NO: 52 - DNA, 50 bp, Artificial sequence
  Single strand DNA oligonucleotide
aaaccctgttcttgggtgggtgtgggtataatactaagttgagatgatat

SEQ ID NO: 53 - DNA, 50 bp, Artificial sequence
  Single strand DNA oligonucleotide
catttacgggggaaggcgctttgtgaagtaggccttatttctcttgtcct

SEQ ID NO: 54 - DNA, 50 bp, Artificial sequence
  Single strand DNA oligonucleotide
  misc_feature (23)..(23): n is a, c, g, or t
ttcgtacagggaggaatttgaangtagatagaaaccgacctggattactc

SEQ ID NO: 55 - DNA, 50 bp, Artificial sequence
  Single strand DNA oligonucleotide
cggtctgaactcagatcacgtaggactttaatcgttgaacaaacgaacct

SEQ ID NO: 56 - DNA, 50 bp, Artificial sequence
  Single strand DNA oligonucleotide
ttaatagcggctgcaccatcgggatgtcctgatccaacatcgaggtcgta

SEQ ID NO: 57 - DNA, 50 bp, Artificial sequence
  Single strand DNA oligonucleotide
aaccctattgttgatatggactctagaataggattgcgctgttatcccta

SEQ ID NO: 58 - DNA, 50 bp, Artificial sequence
  Single strand DNA oligonucleotide
gggtaacttgttccgttggtcaagttattggatcaattgagtatagtagt

SEQ ID NO: 59 - DNA, 50 bp, Artificial sequence
  Single strand DNA oligonucleotide
tcgctttgactggtgaagtcttagcatgtactgctcggaggttgggttct

SEQ ID NO: 60 - DNA, 50 bp, Artificial sequence
  Single strand DNA oligonucleotide
gctccgaggtcgccccaaccgaaatttttaatgcaggtttggtagtttag

SEQ ID NO: 61 - DNA, 50 bp, Artificial sequence
  Single strand DNA oligonucleotide
gacctgtgggtttgttaggtactgtttgcattaataaattaaagctccat

SEQ ID NO: 62 - DNA, 50 bp, Artificial sequence
  Single strand DNA oligonucleotide
agggtcttctcgtcttgctgtgttatgcccgcctcttcacgggcaggtca

SEQ ID NO: 63 - DNA, 50 bp, Artificial sequence
  Single strand DNA oligonucleotide
atttcactggttaaaagtaagagacagctgaaccctcgtggagccattca

SEQ ID NO: 64 - DNA, 50 bp, Artificial sequence
  Single strand DNA oligonucleotide
tacaggtccctatttaaggaacaagtgattatgctacctttgcacggtta

SEQ ID NO: 65 - DNA, 50 bp, Artificial sequence
  Single strand DNA oligonucleotide
gggtaccgcggccgttaaacatgtgtcactgggcaggcggtgcctctaat

SEQ ID NO: 66 - DNA, 50 bp, Artificial sequence
  Single strand DNA oligonucleotide
actggtgatgctagaggtgatgtttttggtaaacaggcggggtaagattt

SEQ ID NO: 67 - DNA, 50 bp, Artificial sequence
  Single strand DNA oligonucleotide
gccgagttccttttactttttttaacctttccttatgagcatgcctgtgt

SEQ ID NO: 68 - DNA, 50 bp, Artificial sequence
  Single strand DNA oligonucleotide
tgggttgacagtgagggtaataatgacttgttggttgattgtagatattg

SEQ ID NO: 69 - DNA, 50 bp, Artificial sequence
  Single strand DNA oligonucleotide
ggctgttaattgtcagttcagtgttttaatctgacgcaggcttatgcgga

SEQ ID NO: 70 - DNA, 50 bp, Artificial sequence
  Single strand DNA oligonucleotide
ggagaatgttttcatgttacttatactaacattagttcttctatagggtg

SEQ ID NO: 71 - DNA, 50 bp, Artificial sequence
  Single strand DNA oligonucleotide
atagattggtccaattgggtgtgaggagttcagttatatgtttgggattt

SEQ ID NO: 72 - DNA, 50 bp, Artificial sequence
  Single strand DNA oligonucleotide
tttaggtagtgggtgttgagcttgaacgctttcttaattggtggctgctt

SEQ ID NO: 73 - DNA, 50 bp, Artificial sequence
  Single strand DNA oligonucleotide
ttaggcctactatgggtgttaaattttttactctctctacaaggtttttt

SEQ ID NO: 74 - DNA, 50 bp, Artificial sequence
  Single strand DNA oligonucleotide
cctagtgtccaaagagctgttcctctttggactaacagttaaatttacaa

SEQ ID NO: 75 - DNA, 49 bp, Artificial sequence
  Single strand DNA oligonucleotide
gggatttagagggttctgtgggcaaatttaaagttgaactaagattcta

SEQ ID NO: 76 - DNA, 50 bp, Artificial sequence
  Single strand DNA oligonucleotide
tcttggacaaccagctatcaccaggctcggtaggtttgtcgcctctacct

SEQ ID NO: 77 - DNA, 50 bp, Artificial sequence
  Single strand DNA oligonucleotide
ataaatcttcccactattttgctacatagacgggtgtgctcttttagctg

SEQ ID NO: 78 - DNA, 50 bp, Artificial sequence
  Single strand DNA oligonucleotide
ttcttaggtagctcgtctggtttcgggggtcttagctttggctctccttg

SEQ ID NO: 79 - DNA, 50 bp, Artificial sequence
  Single strand DNA oligonucleotide
caaagttatttctagttaattcattatgcagaaggtataggggttagtcc

SEQ ID NO: 80 - DNA, 50 bp, Artificial sequence
  Single strand DNA oligonucleotide
ttgctatattatgcttggttataatttttcatctttcccttgcggtacta

SEQ ID NO: 81 - DNA, 50 bp, Artificial sequence
  Single strand DNA oligonucleotide
tatctattgcgccaggtttcaatttctatcgcctatactttatttgggta

SEQ ID NO: 82 - DNA, 50 bp, Artificial sequence
  Single strand DNA oligonucleotide
aatggtttggctaaggttgtctggtagtaaggtggagtgggtttggggct

SEQ ID NO: 83 - DNA, 50 bp, Artificial sequence
  Single strand DNA oligonucleotide
gttcgtccaagtgcactttccagtacacttaccatgttacgacttgtctc

SEQ ID NO: 84 - DNA, 50 bp, Artificial sequence
  Single strand DNA oligonucleotide
ctctatataaatgcgtaggggttttagttaaatgtcctttgaagtatact

SEQ ID NO: 85 - DNA, 50 bp, Artificial sequence
  Single strand DNA oligonucleotide
tgaggagggtgacgggcggtgtgtacgcgcttcagggccctgttcaacta

SEQ ID NO: 86 - DNA, 50 bp, Artificial sequence
  Single strand DNA oligonucleotide
agcactctactcttagtttactgctaaatccaccttcgacccttaagttt

SEQ ID NO: 87 - DNA, 50 bp, Artificial sequence
  Single strand DNA oligonucleotide
cataagggctatcgtagttttctggggtagaaaatgtagcccatttcttg

SEQ ID NO: 88 - DNA, 50 bp, Artificial sequence
  Single strand DNA oligonucleotide
ccacctcatgggctacaccttgacctaacgtctttacgtgggtacttgcg

SEQ ID NO: 89 - DNA, 50 bp, Artificial sequence
  Single strand DNA oligonucleotide
cttactttgtagccttcatcagggtttgctgaagatggcggtatataggc

SEQ ID NO: 90 - DNA, 50 bp, Artificial sequence
  Single strand DNA oligonucleotide
tgagcaagaggtggtgaggttgatcggggtttatcgattacagaacaggc

SEQ ID NO: 91 - DNA, 50 bp, Artificial sequence
  Single strand DNA oligonucleotide
tcctctagagggatatgaagcaccgccaggtcctttgagttttaagctgt

SEQ ID NO: 92 - DNA, 50 bp, Artificial sequence
  Single strand DNA oligonucleotide
ggctcgtagtgttctggcgagcagttttgttgatttaactgttgaggttt

SEQ ID NO: 93 - DNA, 50 bp, Artificial sequence
  Single strand DNA oligonucleotide
agggctaagcatagtggggtatctaatcccagtttgggtcttagctattg

SEQ ID NO: 94 - DNA, 50 bp, Artificial sequence
  Single strand DNA oligonucleotide
tgtgttcagatatgttaaagccactttcgtagtctattttgtgtcaactg

SEQ ID NO: 95 - DNA, 50 bp, Artificial sequence
  Single strand DNA oligonucleotide
gagttttttacaactcaggtgagttttagctttattggggagggggtgat

SEQ ID NO: 96 - DNA, 50 bp, Artificial sequence
  Single strand DNA oligonucleotide
ctaaaacactctttacgccggcttctattgacttgggttaatcgtgtgac

SEQ ID NO: 97 - DNA, 50 bp, Artificial sequence
  Single strand DNA oligonucleotide
cgcggtggctggcacgaaattgaccaaccctggggttagtatagcttagt

SEQ ID NO: 98 - DNA, 48 bp, Artificial sequence
  Single strand DNA oligonucleotide
taaactttcgtttattgctaaaggttaatcactgctgtttcccgtggg

SEQ ID NO: 99 - DNA, 50 bp, Artificial sequence
  Single strand DNA oligonucleotide
tgtggctaggctaagcgttttgagctgcattgctgcgtgcttgatgcttg

SEQ ID NO: 100 - DNA, 50 bp, Artificial sequence
  Single strand DNA oligonucleotide
ttccttttgatcgtggtgatttagagggtgaactcactggaacggggatg

SEQ ID NO: 101 - DNA, 50 bp, Artificial sequence
  Single strand DNA oligonucleotide
cttgcatgtgtaatcttactaagagctaatagaaaggctaggaccaaacc

SEQ ID NO: 102 - DNA, 50 bp, Artificial sequence
  Single strand DNA oligonucleotide
aaagcctacagcacccggtattcccaggcggtctcccatccaagtactaa

SEQ ID NO: 103 - DNA, 50 bp, Artificial sequence
  Single strand DNA oligonucleotide
ccaggcccgaccctgcttagcttccgagatcagacgagatcgggcgcgtt

SEQ ID NO: 104 - DNA, 50 bp, Artificial sequence
  Single strand DNA oligonucleotide
ttccgagatcagacgagatcgggcgcgttcagggtggtatggccgtagac